(19) United States
(12) Patent Application Publication
(10) Pub. No.: US 2003/0065140 A1
(43) Pub. Date: Apr. 3, 2003

(54) Novel proteins and nucleic acids encoding same

(76) Inventors: Corine A.M. Vernet, Branford, CT (US); Catherine E. Burgess, Wethersfield, CT (US); Elma R. Fernandes, Branford, CT (US); Raymond J. Taupier JR., East Haven, CT (US); Kerry E. Quinn, Hamden, CT (US); Kimberly A. Spytek, New Haven, CT (US); Luca Rastelli, Guilford, CT (US); John L. Herrmann, Guilford, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 09/825,751

(22) Filed: Apr. 3, 2001

Related U.S. Application Data
(60) Provisional application No. 60/194,314, filed on Apr. 3, 2000.
(60) Provisional application No. 60/225,693, filed on Aug. 16, 2000.

(51) Int. Cl. C12Q 1/68; C07H 21/04; C07K 14/435; C12P 21/02; C12N 5/06

(52) U.S. Cl. 530/350; 435/69.1; 435/325; 435/320.1; 536/23.5

(57) ABSTRACT

Disclosed herein are novel human nucleic acid sequences which encode polypeptides. Also disclosed are polypeptides encoded by these nucleic acid sequences, and antibodies which immunospecifically-bind to the polypeptide, as well as derivatives, variants, mutants, or fragments of the aforementioned polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from Provisional Applications U.S. S .No. 60/194,314, filed Apr. 3, 2000; and U.S. S. No. 60/225,693, filed Aug. 16, 2000, each of which is incorporated by reference in its entirety.

### FIELD OF THE INVENTION

The invention generally relates to novel AMF1, AMF2, AMF3, AMF4, AMF5, AMF6, AMF7, AMF8, AMF9 and AMF1 nucleic acids and polypeptides encoded therefrom. More specifically, the invention relates to nucleic acids encoding novel polypeptides, as well as vectors, host cells, antibodies, and recombinant methods for producing these nucleic acids and polypeptides.

### BACKGROUND

A need exists for diagnosis, prognosis, and prophylactic or therapeutic treatments of disorders and diseases whose underlying mechanism relates to cell-cell interactions via molecules expressed on the cell surface. Such diseases and disorders include those related to the modulation of cell movement, cell signal processing, cell adhesion or cell migration pathways, including, but not limited to, tissue remodeling, proliferative diseases, cancer, tumor invasion and metastasis, developmental processes, connective tissue regulation, and effects of other extracellular microenvirons. This invention provides methods and compositions to fill this need.

### SUMMARY OF THE INVENTION

The invention is based in part upon the discovery of novel nucleic acid sequences encoding novel polypeptides. The disclosed AMF1, AMF2, AMF3, AMF4, AMF5, AMF6, AMF7, AMF8, AMF9 and AMF10 nucleic acids and polypeptides encoded therefrom, as well as derivatives, homologs, analogs and fragments thereof, will hereinafter be collectively designated as “AMFX” nucleic acid or polypeptide sequences.

In one aspect, the invention provides an isolated AMFX nucleic acid molecule encoding a AMFX polypeptide that includes a nucleic acid sequence that has identity to the nucleic acids disclosed in SEQ ID NOS: 1, 3, 5, 7, 9, 11, 13, 15, 17 and 19. In some embodiments, the AMFX nucleic acid molecule will hybridize under stringent conditions to a nucleic acid sequence complementary to a nucleic acid molecule that includes a protein-coding sequence of a AMFX nucleic acid sequence. The invention also includes an isolated nucleic acid that encodes a AMFX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18 and 20. The nucleic acid can be, for example, a genomic DNA fragment or a cDNA molecule that includes the nucleic acid sequence of any of SEQ ID NOS: 1, 3,5, 7, 9, 11, 13, 15, 17 and 19.

Also included in the invention is an oligonucleotide, e.g., an oligonucleotide which includes at least 6 contiguous nucleotides of a AMFX nucleic acid (e.g., SEQ ID NOS: 1, 3, 5, 7, 9, 11, 13, 15, 17 and 19) or a complement of said oligonucleotide.

Also included in the invention are substantially purified AMFX polypeptides (SEQ ID NO: 2, 4, 6, 8, 10, 12, 14, 16, 18 and 20). In certain embodiments, the AMFX polypeptides include an amino acid sequence that is substantially identical to the amino acid sequence of a human AMFX polypeptide.

The invention also features antibodies that immunoselectively-binds to AMFX polypeptides, or fragments, homologs, analogs or derivatives thereof. In one embodiment of the invention, the anti-AMFX antibody is polyclonal. In another embodiment of the invention, the anti-AMFX antibody is monoclonal. In other embodiments of the invention, the anti-AMFX antibody is therapeutic.

In another aspect, the invention includes pharmaceutical compositions that include therapeutically- or prophylactically-effective amounts of a therapeutic and a pharmaceutically-acceptable carrier. The therapeutic can be, e.g., a AMFX nucleic acid, a AMFX polypeptide, or an antibody specific for a AMFX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a AMFX nucleic acid, under conditions allowing for expression of the AMFX polypeptide encoded by the DNA. If desired, the AMFX polypeptide can then be recovered.

In another aspect, the invention includes a method of detecting the presence of a AMFX polypeptide in a sample. In the method, a sample is contacted with a compound that selectively binds to the polypeptide under conditions allowing for formation of a complex between the polypeptide and the compound. The complex is detected, if present, thereby identifying the AMFX polypeptide within the sample.

The invention also includes methods to identify specific cell or tissue types based on their expression of a AMFX.

Also included in the invention is a method of detecting the presence of a AMFX nucleic acid molecule in a sample by contacting the sample with a AMFX nucleic acid probe or primer, and detecting whether the nucleic acid probe or primer bound to a AMFX nucleic acid molecule in the sample.

In a further aspect, the invention provides a method for modulating the activity of a AMFX polypeptide by contacting a cell sample that includes the AMFX polypeptide with a compound that binds to the AMFX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a Therapeutic in the manufacture of a medicament for treating or preventing disorders or syndromes including, e.g., disorders related to cell signal processing, cell adhesion or migration pathway modulation, including, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli. In contemplated embodiments, successful targeting of AMFX polypeptides using an anti-AMFX monoclonal antibody is anticipated to have an inhibitory effect on tumor growth, and other AMFX-related diseases and disorders. The Therapeutic can be, e.g., a AMFX nucleic acid, a AMFX polypeptide, or a AMFX-specific antibody, or biologically-active derivatives or fragments thereof.

The invention further includes a method for screening for a modulator of disorders or syndromes including, e.g., disorders related to cell signal processing, cell adhesion or migration pathway modulation, including, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli. The method includes contacting a test compound with a AMFX polypeptide and determining if the test compound binds to said AMFX polypeptide. Binding of the test compound to the AMFX polypeptide indicates the test compound is a modulator of activity, or of latency or predisposition to the aforementioned disorders or syndromes. In one embodiment, the test compound is a anti-AMFX antibody.

Also within the scope of the invention is a method for screening for a modulator of activity, or of latency or predisposition to an disorders or syndromes including, e.g., disorders related to cell signal processing, cell adhesion or migration pathway modulation, including, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli, by administering a test compound to a test animal at increased risk for the aforementioned disorders or syndromes. The test animal expresses a recombinant polypeptide encoded by a AMFX nucleic acid. Expression or activity of AMFX polypeptide is then measured in the test animal, as is expression or activity of the protein in a control animal which recombinantly-expresses AMFX polypeptide and is not at increased risk for the disorder or syndrome. Next, the expression of AMFX polypeptide in both the test animal and the control animal is compared. A change in the activity of AMFX polypeptide in the test animal relative to the control animal indicates the test compound is a modulator of latency of the disorder or syndrome.

In yet another aspect, the invention includes a method for determining the presence of or predisposition to a disease associated with altered levels of a AMFX polypeptide, a AMFX nucleic acid, or both, in a subject (e.g., a human subject). The method includes measuring the amount of the AMFX polypeptide in a test sample from the subject and comparing the amount of the polypeptide in the test sample to the amount of the AMFX polypeptide present in a control sample. An alteration in the level of the AMFX polypeptide in the test sample as compared to the control sample indicates the presence of or predisposition to a disease in the subject. Preferably, the predisposition includes, e.g., disorders related to cell signal processing, cell adhesion or migration pathway modulation, including, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a AMFX polypeptide, a AMFX nucleic acid, or a AMFX-specific antibody to a subject (e.g., a human subject), in an amount sufficient to alleviate or prevent the pathological condition. In preferred embodiments, the disorder, including, e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION

The invention is based, in part, upon the discovery of novel nucleic acid sequences that encode novel polypeptides. The novel nucleic acids and their encoded polypeptides are referred to individually as AMF1, AMF2, AMF3, AMF4, AMF5, AMF6, AMF7, AMF8, AMF9 and AMF10. The nucleic acids, and their encoded polypeptides, are collectively designated herein as “AMFX”.

The novel AMFX nucleic acids of the invention include the nucleic acids whose sequences are provided in Tables 1A, 2A, 3A, 4A, 5A, 6A, 7A, 8A, 9A and 1 OA inclusive, or a fragment, derivative, analog or homolog thereof. The novel AMFX proteins of the invention include the protein fragments whose sequences are provided in Tables 1B, 2B, 3B, 4B, 5B, 6B, 7B, 8A, 9A and 10A inclusive. The individual AMFX nucleic acids and proteins are described below. Within the scope of this invention is a method of using these nucleic acids and peptides in the treatment or prevention of a disorder related to cell signal processing, cell adhesion or migration pathway modulation.

AMF-1 (also referred to as Acc. No. 14209510.0.216)

Novel AMF1 is a fibrillin-like protein. The AMF1 clone is alternatively referred to herein as Acc. No. 14209510.0.216. The AMF1 nucleic acid (SEQ ID NO:1) of 1852 nucleotides is shown in Table 1A. The AMF1 open reading frame (“ORF”) begins at nucleotides 208-210. The AMFl ORF terminates at a TGA codon at nucleotides 1699-1701. In one embodiment, the AMF1 polypeptide is a C-terminal fragment, WHEREIN it is contemplated that the AMF1 ORF extends beyond the N-terminus shown in Table 1A, i.e., the sequence demarcated by the solid underline is intron sequence that is later spliced out when the mature full length mRNA is formed. In an alternative embodiment, the AMF1 ORF begins at the in-frame ATG start codon at position 472-474 of SEQ ID NO: 1. In this alternative embodiment, the 5′ UT sequence (demarcated by the solid and dashed underline) would extend to this ATG. As shown in Table 1A, putative 5′ intron region (or alternatively, the 5′ untranslated regions) and the putative untranslated region 3′ to the stop codon are underlined, and the putative start and stop codons are in bold letters.

TABLE 1A
AMF1 nucleotide sequence (SEQ ID NO:1).

CGGATGACTCCCGAGAAGGTGAGCCCCTCACCCACATGCTAAGAGCCCCTTCTGGGCCACCCAGATCCATCTCCGC
ACTGCCTGGGTCTCTGAGTTTCAGGCTCCCCCTGAGAGCCTGGGTGGCCCTGGACCCTGCCAGCCTGGGGCTTGGG
CTTTTGTCCCCTTGGGGCCTTGAGTGTGGCCAGGGCTCTGGCGATTGTGTGGTGACAGAAGCCATGTCTGCAACGC
CTGCCATCCGCAGACGTGAATGAGTGTGCAGAGAACCCTGGCGTCTGCACTAACGGCGTCTGTGTCAACACCGATG
GATCCTTCCGCTGTGAGTGTCCCTTTGGCTACAGCCTGGACTTCACTGGCATCAACTGTGTGGACACAGACGAGTG
CTCTGTCGGCCACCCCTGTGGGCAAGGGACATGCACCAATGTCATCGGAGGCTTCGAATGTGCCTGTGCTGACGGC
TTTGAGCCTGGCCTCATGATGACCTGCGAGGACATCGACGAATGCTCCCTGAACCCGCTGCTCTGTGCCTTCCGCT
GCCACAATACCGAGGGCTCCTACCTGTGCACCTGTCCAGCCGGCTACACCCTGCGGGAGGACGGGGCCATGTGTCG
AGATGTGGACGAGTGTGCAGATGGTCAGCAGGACTGCCACGCCCGGGGCATGGAGTGCAAGAACCTCATCGGTACC
TTCGCGTGCGTCTGTCCCCCAGGCATGCGGCCCCTGCCTGGCTCTGGGGAGGGCTGCACAGATGACAATGAATGCC
ACGCTCAGCCTGACCTCTGTGTCAACGGCCGCTGTGTCAACACCGCGGGCAGCTTCCGGTGCGACTGTGATGAGGG
ATTCCAGCCCAGCCCCACCCTTACCGAGTGCCACGACATCCGGCAGGGGCCCTGCTTTGCCGAGGTGCTGCAGACC
ATGTGCCGGTCTCTGTCCAGCAGCAGTGAGGCTGTCACCAGGGCCGAGTGCTGCTGTGGGGGTGGCCGGGGCTGGG
GGCCCCGCTGCGAGCTCTGTCCCCTGCCCGGCACCTCTGCCTACAGGAAGCTGTGCCCCCATGGCTCAGGCTACAC
TGCTGAGGGCCGAGATGTAGATGAATGCCGTATGCTTGCTCACCTGTGTGCTCATGGGGAGTGCATCAACAGCCTT
GGCTCCTTCCGCTGCCACTGTCAGGCCGGGTACACACCGGATGCTACTGCTACTACCTGCCTGGATATGGATGAGT
GCAGCCAGGTCCCCAAGCCATGTACCTTCCTCTGCAAAAACACGAAGGGCAGTTTCCTGTGCAGCTGTCCCCGAGG
CTACCTGCTGGAGGAGGATGGCAGGACCTGCAAAGACCTGGACGAATGCACCTCCCGGCAGCACAACTGTCAGTTC
CTCTGTGTCAACACTGTGGGCGCCTTCACCTGCCGCTGTCCACCCGGCTTCACCCAGCACCACCAGGCCTGCTTCG
ACAATGATGAGTGCTCAGCCCAGCCTGGCCCATGTGGTGCCCACGGGCACTGCCACAACACCCCGGGCAGCTTCCG
CTGTGAATGCCACCAAGGCTTCACCCTGGTCAGCTCAGGCCATGGCTGTGAAGATGTGAATGAATGTGATGGGCCC
CACCGCTGCCAGCATGGCTGTCAGAACCAGCTAGGGGGCTACCGCTGCAGCTGCCCCCAGGGTTTCACCCAGCACT
CCCAGTGGGCCCAGTGTGTGGGTGAGTGAAAAGGGCTGGGAAGAAGCTGGGCCCTCCACCAGAATCTGCTCAGAGC
AGGCGACTAACAGACGCCACCCTGCAAGATGATGTGACAAGCACAATTATCTAAAGATTGAACAGGCCAGCCCAGA
AGATGAGAATGAGTGTGCCCTGTCGCCC

The 497 aa AMF1 protein (SEQ ID NO:2), is shown in Table 1B. In an alternative embodiment, the AMF1 ORF begins at the first in-frame ATG encoding a methionine at position 89 in SEQ ID NO:2, shown bolded and underlined in Table 1B.

TABLE 1B
AMF1 amino acid sequence (SEQ ID NO:2).

QKPCLQRLPSADVNECAENPGVCTNGVCVNTDGSFRCECPFGYSLDFTGINCVDTDECSVGHPCGQGTCTNVIGGF
ECACADGFEPGLMMTCEDIDECSLNPLLCAFRCHNTEGSYLCTCPAGYTLREDGAMCRDVDECADGQQDCHARGME
CKNLIGTFACVCPPGMRPLPGSGEGCTDDNECHAQPDLCVNGRCVNTAGSFRCDCDEGFQPSPTLTECHDIRQGPC
FAEVLQTMCRSLSSSSEAVTRAECCCGGGRGWGPRCELCPLPGTSAYRKLCPHGSGYTAEGRDVDECRMLAHLCAH
GECINSLGSFRCHCQAGYTPDATATTCLDMDECSQVPKPCTFLCKNTKGSFLCSCPRGYLLEEDGRTCKDLDECTS
RQHNCQFLCVNTVGAFTCRCPPGFTQHHQACFDNDECSAQPGPCGAHGHCHNTPGSFRCECHQGFTLVSSGHGCED
VNECDGPHRCQHGCQNQLGGYRCSCPQGFTQHSQWAQCVGE

In an analysis of public nucleic acid sequence databases, it was found, for example, that the AMF1 nucleic acid sequence has a 238 base fragment with 194 of 238 bases (81%) and a 197 base fragment with 156 of 197 bases (79%) identical to Mus musculus fibrillin 2 (fbn2) gene, complete cds (GenBank Acc. No. L39790) (SEQ ID NO:61) shown in Table 1C. In all BLAST alignments herein, the “E-value” or “Expect” value is a numeric indication of the probability that the aligned sequences could have achieved their similarity to the BLAST query sequence by chance alone, within the database that was searched. For example, as shown in Table IC, the probability that the subject (“Sbjct”) retrieved from the AMF1 BLAST analysis, in this case the Mus musculus fibrillin 2 (fbn2) gene, complete cds, matched the Query AMF1sequence purely by chance is 1 in 9×10²⁶ (i.e., a probability of 9×10²⁶) for the first fragment and 1 in 7×10⁸ for the second fragment.

TABLE 1C
BLASTN of AMF1 against Mus fbn 2 (SEQ ID NOs:61 and 62)

>MUSFBN2 L39790 Mus musculus fibrillin 2 (fbn2) gene, complete cds. 8/1995
Length = 9859, Strand = Plus/Plus
Score = 125 bits (63), Expect = 9e − 26
Identities = 194/238 (81%)
Sbjct: nucleotides 6542-6779 (SEQ ID NO:61)

| | | | | |
|---|---|---|---|---|
| Query: | 293 | tcaacaccgatggatccttccgctgtgagtgtccctttggctacagcctggacttcactg | 352 | (SEQ ID NO:62) |
| | | \|\|\|\|\|\|\| \|\|\|\|\|\|\|\| \|\|\|\|\| \|\|\|\|\|\|\|\|\|\|\| \| \|\|\|\|\|\|\| \|\|\|\|\|\| \| \|\|\|\|\| | | |
| Sbjct: | 6542 | tcaacactgatggatctttccgatgtgagtgtccaatgggctacaacctggattacactg | 6601 | |
| Query: | 353 | gcatcaactgtgtggacacagacgagtgctctgtcggccacccctgtgggcaagggacat | 412 | |
| | | \| \|\| \|\|\|\|\|\|\|\|\|\|\| \|\|\|\|\|\|\|\|\|\|\| \|\|\|\|\| \|\|\|\| \|\| \|\|\| \| \|\|\|\|\|\|\| | | |
| Sbjct: | 6602 | gagtccggtgtgtggacactgacgagtgctccatcggcaacccntgcgggaacgggacat | 6661 | |
| Query: | 413 | gcaccaatgtcatcggaggcttcgaatgtgcctgtgctgacggctttgagcctggcctca | 472 | |
| | | \|\|\|\|\|\|\| \|\| \|\|\|\|\| \|\|\|\|\|\|\|\|\|\| \|\|\|\| \|\| \|\|\|\|\|\|\|\|\|\|\| \|\| \| \|\| | | |
| Sbjct: | 6662 | gcaccaacgtgatcgggtgcttcgaatgcacctgcaacgaaggctttgagccggggccca | 6721 | |
| Query: | 473 | tgatgacctgcgaggacatcgacgaatgctccctgaacccgctgctctgtgccttccg | 530 | |
| | | \|\|\|\|\|\| \|\|\|\|\|\| \|\|\|\|\|\| \|\|\|\| \|\| \|\|\| \|\|\|\|\|\|\|\|\|\|\|\|\|\|\|\|\|\| \|\|\|\|\| | | |
| Sbjct: | 6722 | tgatgaactgcgaagacatcaacgagtgtgcccagaacccgctgctctgtgctttccg | 6779 | |

Strand = Plus/Plus
Score = 65.9 bits (33), Expect = 7e − 08
Identities = 156/197 (79%)
Sbjct: nucleotides 7477-7673 (SEQ ID NO:62)

| | | | |
|---|---|---|---|
| Query: | 1231 | aagccatgtaccttcctctgcaaaaacacgaagggcagtttcctgtgcagctgtccccga | 1290 |
| | | \|\|\|\|\|\|\|\| \| \|\|\|\| \|\|\|\|\|\|\| \|\|\|\|\| \|\|\|\|\|\|\|\|\|\| \|\| \|\|\|\| \|\|\| \|\| \|\| | |
| Sbjct: | 7477 | aagccatgcaacttcatctgcaagaacaccaagggcagttaccagtgctcctgcccacgg | 7536 |
| Query: | 1291 | ggctacctgctggaggaggatggcaggacctgcaaagacctggacgaatgcacctcccgg | 1350 |
| | | \|\| \|\|\| \| \|\|\| \|\|\|\|\|\|\| \|\| \| \|\|\| \|\|\|\|\|\|\|\|\|\|\| \|\|\|\|\|\|\|\| \|\| | |
| Sbjct: | 7537 | gggtacgtcctgcaggaggacggaaagacgtgcaaagacctcgacgaatgtcaaaccaaa | 7596 |
| Query: | 1351 | cagcacaactgtcagttcctctgtgtcaacactgtgggcgccttcacctgccgctgtcca | 1410 |
| | | \|\|\|\|\|\|\|\|\|\|\| \|\|\|\|\|\|\|\|\|\|\|\|\|\|\|\|\|\|\|\| \|\|\|\| \| \|\|\|\|\|\|\|\| \|\|\|\|\| | |
| Sbjct: | 7597 | cagcacaactgccagttcctctgtgtcaacaccctggggggattcacctgtaaatgtccg | 7656 |
| Query: | 1411 | cccggcttcacccagca | 1427 |
| | | \|\|\|\|\| \|\|\|\|\|\|\|\|\|\|\| | |
| Sbjct: | 7657 | cccggtttcacccagca | 7673 |

In addition, the AMF1 nucleic acid sequence has strong homology to other nucleic acids as shown in the BlastN results in Table 1D.

TABLE 1D
BLASTN alignment data of AMF1

| | Score | E |
|---|---|---|
| b Sequences producing significant alignments: | (bits) | Value |
| MUSFBN2 L39790 Mus musculus fibrillin 2 (fbn2) gene, complet . . . | 125 | 9e − 26 |
| MMU20217 U20217 Mus musculus fibrillin-2 mRNA, partial cds . . . | 115 | 9e − 23 |
| HUMFIBRLLN L13923 Homo sapiens fibrillin mRNA, complete cds . . . | 72 | 1e − 09 |
| HSFIBRMR X63556 H.sapiens mRNA for fibrillin. 2/1997 | 72 | 1e − 09 |
| AF187554 AF187554 Homo sapiens sperm antigen-36 mRNA, comple . . . | 72 | 1e − 09 |
| AF135060 AF135060 Rattus norvegicus fibrillin-2 mRNA, comple . . . | 66 | 7e − 08 |
| AF073800 AF073800 Sus scrofa fibrillin-1 precursor (FBN1) mR . . . | 58 | 2e − 05 |
| AF135059 AF135059 Rattus norvegicus fibrillin-1 mRNA, comple . . . | 56 | 7e − 05 |

A BLASTP search was performed against public protein databases. As shown in Table 1E, the AMF1 protein has 137 of 349 amino acid residues (39%) identical to, and 200 of 349 residues (57%) positive with, the 492 amino acid residue long Homo sapiens transmembrane protease, serine 2 (ec 3.4.21.-.) (SEQ ID NO:63).

TABLE 1E
BLASTN of AMF1 against TMS 2 (SEQ ID NO:63)

TMS2_HUMAN homo sapiens transmembrane protease, serine 2 (ec 3.4.21.-). 7/1998
Length = 492
Score = 266.0, bits (673.0), Expect = 1e − 70
Identities = 137/349 (39%), Positives = 200/349, (57%)

| | | | | |
|---|---|---|---|---|
| Query: | 1 | CVRFDWDKSLLKIYSGSSHQWLPICSSNWNDSYSEKTCQQLGFESAHRTTEVAHRDFANS | 60 | (SEQ ID NO: 63) |
| | | \|\|\| +\|++\|\| \| \|+\| +\|\|++\| \|+ +\|+++ +++ \| + \| | | |
| Sbjct: | 148 | CVRLYGPNFILQMYSSQRKSWHPVCQDDWNENYGRAACRDMGYKNNFYSSQGIVDD-SGS | 206 | |
| Query: | 61 | FSILRYNST-----IQESLHRSE-CPSQRYISLQCSHCGLR---AMTGRIVGGALASDSK | 111 | |
| | | \| ++ \|++ \| + \|+ \|+ \| \|+ +\|\|+\| \|\|+ + \|\|\|\|\| \| | | |
| Sbjct: | 207 | TSFMKLNTSAGNVDIYKKLYHSDACSSKAVVSLRCLACGVNLNSSRQSRIVGGESALPGA | 266 | |
| Query: | 112 | WPWQVSLHFGTTHICGGTLIDAQWVLTAAHCFFVTREKVLEG---WKVYAGTSNLHQLPE | 168 | |
| | | \|\|\|\|\|\|\|\| \|+\|\|\|++\| +\|+\|\|\|\|\| \|\| \| \| +\|\| + | | |
| Sbjct: | 267 | WPWQVSLHVQNVHVCGGSIITPEWIVTAAHCV----EKPLNNPWHWTAFAGILRQSFMFY | 322 | |
| Query: | 169 | AAS--IAEIIINSNYTDEEDDYDIALMRLSKPLTLSAHIHPACLPMHGQTFSLNETCWIT | 226 | |
| | | \| + ++\| + \|\| + + \|\|\|\|\|+\| \|\|\|\| + + \| \|\|\| \| + \|\|\|+ | | |
| Sbjct: | 323 | GAGYQVQKVISHPNYDSKTKNNDIALMKLQKPLTFNDLVKPVCLPNPGMMLQPEQLCWIS | 382 | |
| Query: | 227 | GFGKTRETDDKTSPFLREVQVNLIDFKKCNDYLVYDSYLTPRMMCAGDLRGGRDSCQCDS | 286 | |
| | | \|+\| \| \| \|\|\| \| +\| \|\|+ ++\|\| \|\|\| + +\|\| \|+ \|\|\| \| +\| \|\|\|\|\|\|\| | | |
| Sbjct: | 383 | GWGATEEKG-KTSEVLNAAKVLLIETQRCNSRYVYDNLITPAMICAGFLQGNVDSCQGDS | 441 | |
| Query: | 287 | GGPLVCEQNNRWYLAGVTSWGTGCGQRNKPGVYTKVTEVLPWIYSKMES | 335 | |
| | | \|\|\|\|\| \|\| \|+\| \| \|\|\|\|+\|\| + +\|\|\|\| \| \|\|\| +\|++ | | |
| Sbjct: | 442 | GGPLVTSNNNIWWLIGDTSWGSGCAKAYRPGVYGNVMVFTDWTYRQMKA | 490 | |

AMF1 also has high homology to a number of other amino acid sequences as shown in the BLASTP alignment data in Table 1F.

TABLE 1F
BLASTP analysis results for AMF1

| Matching Entry | | | | |
|---|---|---|---|---|
| (in SwissProt + | Begin- | | | E |
| SpTrEMBL) | End | Description | Score | Value |
| TMS2_HUMAN | [1-335] | TRANSMEMBRANE PROTEASE, SERINE 2 (EC | 266.0 | 1e−70 |
| | | 3.4.21.-). | | |
| HEPS_HUMAN | [11-335] | SERINE PROTEASE HEPSIN (EC 3.4.21.-) | 232.0 | 2e−60 |
| | | (TRANSMEMBRANE PROTEASE, SERENE1). | | |
| HEPS_MOUSE | [9-335] | SERINE PROTEASE HEPSIN (EC 3.4.21.-). | 230.0 | 1e−59 |
| HEPS_RAT | [9-340] | SERINE PROTEASE HEPSIN (EC 3.4.21.-). | 224.0 | 8e−58 |
| KAL_HUMAN | [90-335] | PLASMA KALLIKREIN PRECURSOR (EC 3.4.21.34) | 219.0 | 2e−56 |
| | | (PLASMA PREKALLIKREIN)(KININOGENJN) | | |
| | | (FLETCHER FACTOR). | | |
| KAL_MOUSE | [97-335] | PLASMA KALLIKREIN PRECURSOR (EC 3.4.21.34) | 215.0 | 3e−55 |
| | | (PLASMA PREKALLIKREIN)(KININOGENIN) | | |
| | | (FLETCHER FACTOR). | | |
| KAL_RAT | [87-335] | PLASMA KALLIKREIIN PRECURSOR (EC 3.4.21.34) | 213.0 | 2e−54 |
| | | (PLASMA PREKALLIKREIN)(KININOGENIN) | | |
| | | (FLETCHER FACTOR). | | |
| O95518 | [92-329] | DJ1170K4.2 (NOVEL TRYPSIN FAMILY PROTEIN | 213.0 | 2e−54 |
| | | WITH CLASS A LDL RECEPTORDOMAINS) | | |
| | | (FRAGMENT). | | |
| O97506 | [90-336] | ALLIKREIN. | 204.0 | 6e−52 |

The presence of identifiable domains in AMF1, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMFX RNA was derived using tissue sources including, but not limited to, proprietary database sources, public EST sources, literature sources, and/or RACE sources, as described in the Examples. AMF1 is expressed in at least the following tissues: colon, gastric and ovarian cancer derived cell lines. It is also strongly expressed in fetal kidney and lung indicating an oncofetal phenotype.

The nucleic acids and proteins of AMF1 are useful in potential therapeutic applications implicated in various AMF-related pathologies and/or disorders. For example, a cDNA encoding the fibrillin-like protein may be useful in gene therapy, and the fibrillin-like protein may be useful when administered to a subject in need thereof. The novel nucleic acid encoding AMF1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from: colon, gastric, and ovarian cancer, and other diseases, disorders and conditions of the like. By way of nonlimiting example, the compositions of the present invention will have efficacy for treatment of patients suffering from colon, gastric, and ovarian cancer. Additional AMF-related diseases and disorders are mentioned throughout the Specification.

Further, the protein similarity information, expression pattern, and map location for AMF1 suggests that AMF1 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF1 substances for use in therapeutic or diagnostic methods. These antibodies may be generated according to methods known in the art, using prediction from hydrophobicity charts, as described in the “Anti-AMFX Antibodies” section below. In various embodiments, contemplated AMF I epitopes are hydrophilic regions of the AMF I polypeptide as predicted by software programs well known in the art that generate hydrophobicity or hydrophilicity plots.

AMF-2 (Also Referred to as Acc. No. 20421338)

Novel AMF2 is a nephrin-like protein. The AMF2 clone is alternatively referred to herein as Acc No. 20421338. The AMF2 nucleic acid (SEQ ID NO:3) of 379 nucleotides is shown in Table 2A. In one embodiment, the AMF2 construct is an internal fragment of a larger gene, wherein it is contemplated that the ORF extends beyond the N- and C-termini depicted in Tables 2A and 2B. As shown in Table 2A, the first coding triplet beginning at position 1 is in bold letters.

TABLE 2A
AMF2 nucleotide sequence (SEQ ID NO:3).

GGAGGGCCTGTGATTCTACTGCAGGCAGGCACCCCCCACAACCTCACATGCCGGGCCTTCAATGCGAAGCCTGCTG
CACCATCATCTGGTTCCGGGACGGGACGCAGCAGGAGGGCGCTGTGGCCAGCACGGAATTGCTGAAGGATGGGAA
GAGGGAGACCACCGTGAGCCAACTGCTTATTAACCCCACGGACCTGGACATAGGGCGTGTCTTCACTTGCCGAAGC
ATGAACGAAGCCATCCCTAGTGGCAAGGAGACTTCCATCGAGCTGGATGTGCACCACCCTCCTACAGTGACCCTGT
CCATTGAGCCACAGACGGGGCAGGAGGGTGAGCGTGTTGTCTTTACCTGCCAGGCCACAGCCAACCCCGAGATCT

The encoded AMF2 protein (SEQ ID NO:4) of 126 amino acids (SEQ ID NO:4) is shown in Table 2B.

TABLE 2B
AMF2 amino acid sequence (SEQ ID NO:4).

GGPVILLQAGTPHNLTCRAFNAKPAATIIWFRDGTQQEGAVASTELLKDGKRETTVSQLLINPTDLDIGRVFTCRS
MNEAIPSGKETSIELDVHHPPTVTLSIEPQTGQEGERVVFTCQATANPEI

In an analysis of public nucleic acid sequence databases, it was found, for example, that the AMF2 nucleic acid sequence has 162 of 163 bases (99%) identical to a Homo sapiens cDNA FLJ12646 fis, clone NT2RM4001987, weakly similar to Neural Cell Adhesion Molecule 1, Large Isoform Precursor (GenBank Acc. No. AK022708) (SEQ ID NO:64) shown in Table 2C.

TABLE 2C
BLASTN alignment of AMF2 against NT2RM4001987 (SEQ ID NO:64)

>AK022708 AK022708 Homo sapiens cDNA FLJ12646 fis, clone NT2RM4001987, weakly
similar to NEURAL CELL ADHESION MOLECULE 1, LARGE ISOFORM PRECURSOR. 9/2000
Length = 2656
Score = 315 bits (159), Expect = 9e − 84
Identities = 162/163 (99%)
Strand = Plus/Plus

| | | | | |
|---|---|---|---|---|
| Query: | 217 | acttgccgaagcatgaacgaagccatccctagtggcaaggagacttccatcgagctggat | 276 | (SEQ ID NO: 64) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | acttgccgaagcatgaacgaagccatccctagtggcaaggagacttccatcgagctggat | 60 | |
| Query: | 277 | gtgcaccaccctcctacagtgaccctgtccattgagccacagacggggcaggagggtgag | 336 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | gtgcaccaccctcctacagtgaccctgtccattgagccacagacggtgcaggagggtgag | 120 | |
| Query: | 337 | cgtgttgtctttacctgccaggccacagccaaccccgagatct | 379 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | cgtgttgtctttacctgccaggccacagccaaccccgagatct | 163 | |

A BLASTP search was performed against public protein databases. As shown in Table 2D, the AMF2 protein has 36 of 120 amino acid residues (30%) identical to, and 54 of 120 residues (45%) positive with, the 1011 amino acid residue long Drosophila melanogaster (fruit fly) neuromusculin (Acc. No. Q24273) (SEQ ID NO:65).

TABLE 2D
BLASTP of AMF2 against Neuromusculin (SEQ ID NO:65)

>Q24273 Q24273 drosophila melanogaster (fruit fly). neuromusculin. 5/1999
Length = 1011
Score = 55.8 bits (132), Expect = 9e − 08
Identities = 36/120 (30%), Positives = 54/120 (45%), Gaps = 10/120 (8%)

| | | | |
|---|---|---|---|
| Query: | 15 | LTCRAFNAKPAATIIWFR------DGTQQEGAVASTELLKDGKRETTVSQLLINPTDLDI | 68 |
| | | \|\|\| \|+\|\| + \|+ \| + + \| \| \| \| \| \| +\|\| \| \| + | |
| Sbjct: | 282 | LTCDIHGARPAVNLTWYNTTTIISSGENEITEVRSKSLEKSDGTFHTQSELIFNATRFEN | 341 |
| Query: | 69 | GRVFTCRSMNEAIPSGKE----TSIELDVHHPPTVTLSIEPQTGQEGERVVFTCQATANP | 124 |
| | | \|\|\| \| + \| + +\| +++ \|+\| +\|\| \| +\| \| \| \|+ \|+ \|\|\| | |
| Sbjct: | 342 | DRVFRCEAENIVLQINREKPISSALTLEVLYPPVVKVSPSAITANTSEIVLLNCEYFANP | 401 |

AMF2 also has high homology 30 of 114 amino acids (26%) identical and 59 of 114 amino acids (51%) positive with the 862 amino acid protein Mus musculus (mouse) b-cell receptor cd22 precursor (leu-14) (b-lymphocyte cell adhesion molecule) (bl-cam) (Acc. No. P35329)(SEQ ID NO:66). Table 2E.

TABLE 2E
BLASTP of AMF2 against CD22 (SEQ ID NO:66)

>CD22_MOUSE P35329 mus musculus (mouse). b-cell receptor cd22 precursor
(leu-14) (b-lymphocyte
cell adhesion molecule) (bi-cam).
7/19 99
Length = 862
Score = 51.5 bits (121), Expect = 2e − 06
Identities = 30/114 (26%), Positives = 59/114 (51%), Gaps = 13/114 (11%)

| | | | |
|---|---|---|---|
| Query: | 15 | LTCRAFNAKP---AATIIWFRDGTQQEGAVASTELLKDGKRETTVSQLLINPTDLDIGRV | 71 |
| | | +\|\|\| ++ \| + \|\|+\|\| \| ++ ++\| +\|+\|+++ \|+ | |
| Sbjct: | 270 | MTCRVNSSNPKLRTVAVSWFKDGRPLED--------QELEQEQQMSKLILHSVTKDMRGK | 321 |
| Query: | 72 | FTCRSMNEAIPSGKETSIELDVHHPPTVT-LSIEPQTGQEGERVVFTCQATANP | 124 |
| | | + \|++ \|+ \| \|+ +\|\| \|\| \|\|+ \| + + \| \| +\|\|+ \| \|++ \|+\| | |
| Sbjct: | 322 | YRCQASNDIGP-GESEEVELTVHYAPEPSRVHIYPSPAEEGQSVELICESLASP | 374 |

AMF2 also has high homology to other amino acid sequences shown in the BLASTP alignment data in Table 2F.

TABLE 2F
BLASTP alignments of AMF2

| BLASTP | Score | E |
|---|---|---|
| Sequences producing significant alignments: | (bits) | Value |
| Q24273 Q24273 drosophila melanogaster (fruit fly) . neuromusc . . . | 56 | 9e−08 |
| CD22_MOUSE P35329 mus musculus (mouse) . b-cell receptor cd22 . . . | 52 | 2e−06 |
| O97174 O97174 drosophila melanogaster (fruit fly) . eg:163a10 . . . | 50 | 5e−06 |
| Q9Z2H8 Q9z2h8 mus musculus (mouse) . immunosuperfamily protei . . . | 49 | 1e−05 |

The presence of identifiable domains in AMF1, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMF2 RNA was derived using tissue sources including, but not limited to, proprietary database sources, public EST sources, literature sources, and/or RACE sources, as described in the Examples. AMF2 is expressed in at least the following tissues: fetal kidney and several cell lines derived from renal cell carcinomas. It is also upregulated in brain tumor and melanoma derived cell lines.

The nucleic acids and proteins of AMF1 are useful in potential therapeutic applications implicated in various AMF-related pathologies and/or disorders. For example, a cDNA encoding the nephrin-like protein may be useful in gene therapy, and the nephrin-like protein may be useful when administered to a subject in need thereof. The novel nucleic acid encoding AMF2 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMF2 nucleic acids and proteins are useful in potential diagnostic and therapeutic applications implicated in various diseases and disorders described below and/or other pathologies. For example, the compositions of the present invention will have efficacy for treatment of patients suffering from: renal cell carcinoma, brain tumors, melanoma, congenital nephritic syndrome of Finnish type and other diseases, disorders and conditions of the like. By way of nonlimiting example, the compositions of the present invention will have efficacy for treatment of patients suffering from renal cell carcinoma, brain tumors, melanoma, congenital nephritic syndrome of Finnish type. Additional AMF2-related diseases and disorders are mentioned throughout the Specification.

Further, the protein similarity information, expression pattern, and map location for AMF2 suggests that AMF2 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF2 substances for use in therapeutic or diagnostic methods. These antibodies may be generated according to methods known in the art, using prediction from hydrophobicity charts, as described in the “Anti-AMFX Antibodies” section below. In various embodiments, contemplated AMF2 epitopes are hydrophilic regions of the AMF2 polypeptide as predicted by software programs well known in the art that generate hydrophobicity or hydrophilicity plots.

AMF-3 (Also Referred to as Acc. No. 27251385)

Novel AMF3 is a fibrillin-like protein related to the gene. The AMF3 clone is alternatively referred to herein as Acc. No.27251385. The AMF3 nucleic acid (SEQ ID NO:5) of 3374 nucleotides is shown in Table3A. The AMF3 open reading frame (“ORF”) begins at nucleotides 3-5. The AMF3 ORF terminates at a TAG codon at nucleotides 3357-3359. AMF3 appears to be a C-terminal fragment, so it is contemplated that the ORF extends beyond the depicted N-terminus. As shown in Table 3A, putative untranslated regions 5′ to the start codon and 3′ to the stop codon are underlined, and the first coding triplet and stop codon are in bold letters.

TABLE 3A
AMF3 nucleotide sequence (SEQ ID NO:5).

GCCAGGGAGGCAGCTGCGTCAACATGGTGGGCTCCTTCCATTGCCGCTGTCCAGTTGGACACCGGCTCAGTGACAG
CAGCGCCGCATGTGAAGACTACCGGGCCGGCGCCTGCTTCTCAGTGCTTTTCGGGGGCCGCTGTGCTGGAGACCTC
GCCGGCCACTACACTCGCAGGCAGTGCTGCTGTGACAGGGGCAGGTGCTGGGCAGCTGGCCCGGTCCCTGAGCTGT
GTCCTCCTCGGGGCTCCAATGAATTCCAGCAACTGTGCGCCCAGCGGCTGCCGCTGCTACCCGGCCACCCTGGCCT
CTTCCCTGGCCTCCTGGGCTTCGGATCCAATGGCATGGGTCCCCCTCTTGGGCCAGCGCGACTCAACCCCCATGGC
TCTGATGCGCGTGGGATCCCCAGCCTGGGCCCTGGCAACTCTAATATTGGCACTGCTACCCTGAACCAGACCATTG
ACATCTGCCGACACTTCACCAACCTGTGTCTGAATGGCCGCTGCCTGCCCACGCCTTCCAGCTACCGCTGCGAGTG
TAACGTGGGCTACACCCAGGACGTGCGCGGCGAGTGCATTGATGTAGACGAATGCACCAGCAGCCCCTGCCACCAC
GGTGACTGCGTCAACATCCCCGGCACCTACCACTGCCGGTGCTACCCGGGCTTCCAGGCCACGCCCACCAGGCAGG
CATGCGTGGATGTGGACGAGTGCATTGTCAGTGGTGGCCTTTGTCACCTGGGCCGCTGTGTCAACACAGAGGGCAG
CTTCCAGTGTGTCTGCAATGCAGGCTTCGAGCTCAGCCCTGACGGCAAGAACTGTGTGGACCACAACGAGTGTGCC
ACCAGCACCATGTGCGTCAACGGCGTGTGTCTCAACGAGGATGGCAGCTTCTCCTGCCTCTGCAAACCCGGCTTCC
TGCTGGCGCCTGGCGGCCACTACTGCATGGACATTGACGAGTGCCAGACGCCCGGCATCTGCGTGAACGGCCACTG
TACCAACACCGAGGGCTCCTTCCGCTGCCAGTGCCTGGGGGGGCTGGCGGTAGGCACGGATGGCCGCGTGTGCGTG
GACACCCACGTGCGCAGCACCTGCTATGGGGCCATCGAGAAGGGCTCCTGTGCCCGCCCCTTCCCTGGCACTGTCA
CCAAGTCGGAGTGCTGCTGTGCCAATCCGGACCACGGTTTTGGGGAGCCCTGCCAGCTTTGTCCTGCCAAAAACTC
CGCTGAGTTCCAGGCACTGTGCAGCAGTGGGCTTGGCATTACCACGGATGGTCGAGACATCAACGAGTGTGCTCTG
GATCCTGAGGTTTGTGCCAATGGCGTGTGCGAGAACCTTCGGGGCAGCTACCGCTGTGTCTGCAACCTGGGTTATG
AGGCAGGTGCCTCAGGCAAGGACTGCACAGACGTGGATGAGTGTGCCCTCAACAGCCTCCTGTGTGACAACGGGTG
GTGCCAGAATAGCCCTGGCAGCTACAGCTGCTCCTGCCCCCCCGGCTTCCACTTCTGGCAGGACACGGAGATCTGC
AAAGATGTCGACGAATGCCTGTCCAGCCCGTGTGTGAGTGGCGTTTGTCGGAACCTGGCCGGCTCCTACACCTGCA
AATGTGGCCCTGGCAGCCGGCTGGACCCCTCTGGTACCTTCTGTCTAGACAGCACCAAGGGCACCTGCTGGCTGAA
GATCCAGGAGAGCCGCTGTGAGGTGAACCTTCAGGGAGCCAGCCTGCGGTCTGAGTGCTGTGCCACCCTCGGGGCA
GCCTGGGGGAGCCCCTGCGAACGCTGCGAGATCGACCCTGCCTGTGCCCGGGGCTTTGCCCGGATGACGGGTGTCA
CCTGCGATGATGTGAACGAGTGTGAGTCCTTCCCGGGAGTCTGTCCCAACGGGCGTTGCGTCAACACTGCTGGGTC
TTTCCGCTGTGAGTGTCCAGAGGGCCTGATGCTGGACGCCTCAGGCCGGCTGTGCGTGGATGTGAGATTGGAACCA
TGTTTCCTGCGATGGGATGAGGATGAGTGTGGGGTCACCCTGCCTGGCAAGTACCGGATGGACGTCTGCTGCTGCT
CCATCGGGGCCGTGTGGGGAGTCGAGTGCGAGGCCTGCCCGGATCCCGAGTCTCTGGAGTTCGCCAGCCTGTGCCC
GCGGGGGCTGGGCTTCGCCAGCCGGGACTTCCTGTCTGGCCGACCATTCTATAAAGATGTGAATGAATGCAAGGTG
TTCCCTGGCCTCTGCACGCACGGTACCTGCAGAAACACGGTGGGCAGCTTCCACTGCGCCTGTGCGGGGGGCTTCG
CCCTGGATGCCCAGGAACGGAACTGCACAGATATCGACGAGTGTCGCATCTCTCCTGACCTCTGCGGCCAGGGCAC
CTGTGTCAACACGCCGGGCAGCTTTGAGTGCGAGTGTTTTCCCGGCTACGAGAGTGGCTTCATGCTGATGAAGAAC
TGCATGGACGTGGACGAGTGTGCAAGGGACCCGCTGCTCTGCCGGGGAGGCACTTGCACCAACACGGATGGGAGCT
ACAAGTGCCAGTGTCCCCCTGGGCATGAGCTGACGGCCAAGGGCACTGCCTGTGAGGACATCGATGAGTGCTCCCT
GAGTGATGGCCTGTGTCCCCATGGCCAGTGTGTCAATGTCATCGGTGCCTTCCAGTGCTCCTGCCATGCCGGCTTC
CAGAGCACACCTGACCGCCAGGGCTGCGTGGACATCAACGAATGCCGGGTCCAGAATGGTGGGTGTGACGTGCACC
GTATTAACACTGAGGGCAGCTACCGGTGCAGCTGTGGGCAGGGCTACTCGCTGATGCCCGACGGAAGGGCATGTGC
AGACGTGGACGAGTGTGAAGAGAACCCCCGCGTTTGTGACCAAGGCCACTGCACCAACATGCCAGGGGGTCACCGC
TGCCTGTGCTATGATGGCTTCATGGCCACGCCAGACATGAGGACATGTGTTGATGTGGATGAGTGTGACCTGAACC
CTCACATCTGCCTCCATGGGGACTGCGAGAACACGAAGGGTTCCTTTGTCTGCCACTGTCAGCTGGGCTACATGGT
CAGGAAGGGGGCCACAGGCTGCTCTGATGTGGATGAATGCGAGGTTGGAGGACACAACTGTGACAGTCACGCCTCC
TGTCTCAACATCCCGGGGAGTTTCAGCTGTAGGTGCCTGCCAGGCTGGGTGGGGGATGGCTTCGAATGTCACGACC
TGGATGAATGCGTCTCCCAGGAGCACCGGTGCAGCCCAAGAGGTGACTGTCTCAATGTCCCTGGCTCCTACCGCTG
CACCTGCCGCCAGGGCTTTGCCGGGGATGGCTTCTTCTGCGAAGACAGGGATGAATGTGCCGAGAACGTGGACCTC
TGTGACAACGGGTAGTGCCTCAATGCGCCC

The encoded AMF3 protein (SEQ ID NO:6) of 1118 amino acids (SEQ ID NO:6) is shown in Table 3B.

TABLE 3B
AMF3 amino acid sequence (SEQ ID NO:6)

QGGSCVNMVGSFHCRCPVGHRLSDSSAACEDYRAGACFSVLFGGRCAGDLAGHYTRRQCCCDRGRCWAAGPVPELC
PPRGSNEFQQLCAQRLPLLPGHPGLFPGLLGFGSNGMGPPLGPARLNPHGSDARGIPSLGPGNSNIGTATLNQTID
ICRHFTNLCLNGRCLPTPSSYRCECNVGYTQDVRGECIDVDECTSSPCHHGDCVNIPGTYHCRCYPGFQATPTRQA
CVDVDECIVSGGLCHLGRCVNTEGSFQCVCNAGFELSPDGKNCVDHNECATSTMCVNGVCLNEDGSFSCLCKPGFL
LAPGGHYCMDIDECQTPGICVNGHCTNTEGSFRCQCLGGLAVGTDGRVCVDTHVRSTCYGAIEKGSCARPFPGTVT
KSECCCANPDHGFGEPCQLCPAKNSAEFQALCSSGLGITTDGRDINECALDPEVCANGVCENLRGSYRCVCNLGYE
AGASGKDCTDVDECALNSLLCDNGWCQNSPGSYSCSCPPGFHFWQDTEICKDVDECLSSPCVSGVCRNLAGSYTCK
CGPGSRLDPSGTFCLDSTKGTCWLKIQESRCEVNLQGASLRSECCATLGAAWGSPCERCEIDPACARGFARMTGVT
CDDVNECESFPGVCPNGRCVNTAGSFRCECPEGLMLDASGRLCVDVRLEPCFLRWDEDECGVTLPGKYRMDVCCCS
IGAVWGVECEACPDPESLEFASLCPRGLGFASRDFLSGRPFYKDVNECKVFPGLCTHGTCRNTVGSFHCACAGGFA
LDAQERNCTDIDECRISPDLCGQGTCVNTPGSFECECFPGYESGFMLMKNCMDVDECARDPLLCRGGTCTNTDGSY
KCQCPPGHELTAKGTACEDIDECSLSDGLCPHGQCVNVIGAFQCSCHAGFQSTPDRQGCVDINECRVQNGGCDVHR
INTEGSYRCSCGQGYSLMPDGRACADVDECEENPRVCDQGHCTNMPGGHRCLCYDGFMATPDMRTCVDVDECDLNP
HICLHGDCENTKGSFVCHCQLGYMVRKGATGCSDVDECEVGGHNCDSHASCLNIPGSFSCRCLPGWVGDGFECHDL
DECVSQEHRCSPRGDCLNVPGSYRCTCRQGFAGDGFFCEDRDECAENVDLCDNG

In an analysis of public nucleic acid sequence databases, it was found, for example, that a fragment of the AMF3 nucleic acid sequence has 134 of 134 bases (100%) identical to a Homo sapiens cDNA FLJ20029 fis, clone ADSE02022 (GenBank Acc. No. AK000036) (SEQ ID NO:67) shown in Table 3C.

TABLE 3C
BLASTN of AMF3 against FLJ20029 (SEQ ID NO:67)

>AK000036 AK000036 Homo sapiens cDNA FLJ20029 fis, clone ADSE02022. 2/2000
Length = 1399; Strand = Plus/Plus
Score = 266 bits (134), Expect = 7e-68
Identities = 134/134 (100%)

| | | | |
|---|---|---|---|
| Query: | 2306 | cacagatatcgacgagtgtcgcatctctcctgacctctgcggccagggcacctgtgtcaa | 2365 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 190 | cacagatatcgacgagtgtcgcatctctcctgacctctgcggccagggcacctgtgtcaa | 249 |
| Query: | 2366 | cacgccgggcagctttgagtgcgagtgttttcccggctacgagagtggcttcatgctgat | 2425 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 250 | cacgccgggcagctttgagtgcgagtgttttcccggctacgagagtggcttcatgctgat | 309 |
| Query: | 2426 | gaagaactgcatgg 2439 | |
| | | \|\|\|\|\|\|\|\|\|\|\|\|\|\| | |
| Sbjct: | 310 | gaagaactgcatgg 323 | |

In addition, the AMF3 nucleic acid sequence has high homology to other nucleic acid sequences, as shown in BLASTN alignment data in Table 3D.

TABLE 3D
BLASTN alignment results for AMF3

| | Score | E |
|---|---|---|
| Sequences producing significant alignments: | (bits) | Value |
| AK000036 AK000036 Homo sapiens cDNA FLJ20029 fis, clone ADSE. . . | 266 | 7e−68 |
| AF135060 AF135060 Rattus norvegicus fibrillin-2 mRNA, comple . . . | 125 | 2e−25 |
| MUSFBN2 L39790 Mus musculus fibrillin 2 (fbn2) gene, complet . . . | 109 | 1e−20 |
| HSU03272 U03272 Human fibrillin-2 mRNA, complete cds. 6/1994 | 98 | 4e−17 |
| HSFIB5 X62009 Homo sapiens partial mRNA for fibrillin 5. 9/1999 | 98 | 4e−17 |
| AC025169 AC025169 Homo sapiens chromosome 5 clone CTC-352M6, . . . | 90 | 9e−15 |
| AC010461 AC010461 Homo sapiens chromosome 5 clone CTD-2275A5 . . . | 90 | 9e−15 |

A BLASTP search was performed against public protein databases. As shown in Table 3E, the AMF3 protein has 766 of 1178 amino acid residues (65%) identical to, and 913 of 1178 amino acid residues (77%) positive with, the 2911 amino acid residue long Homo sapiens (human). fibrillin 2 precursor (Acc. No. P35556) (SEQ ID NO:68).

TABLE 3E
BLASTP of AMF3 against FBN2 (SEQ ID NO:68)

>FBN2_HUMAN P35556 homo sapiens (human). fibrillin 2 precursor. 11/1997
Length = 2911
Score = 1804 bits (4622), Expect = 0.0
Identities = 766/1178 (65%), Positives = 913/1178 (77%), Gaps = 62/1178 (5%)

| | | | |
|---|---|---|---|
| Query: | 1 | QGGSCVNMVGSFHCRCPVGHRLSDSSAACE------------------------------ | 30 |
| | | \|\|\|+\|+\| \|\|\|\| \|\|\|\| \|\|+ \|+++ \|\| | |
| Sbjct: | 287 | QGGNCINTVGSFECRCPAGHKQSETTQKCEDIDECSIIPGICETGECSNTVGSYFCVCPR | 346 |
| Query: | 31 | ------------DYRAGACFSVLFGGRCAGDLAGHYTRRQCCCDRGRCWAAGPVPELCPP | 78 |
| | | \| \| \| \|\|\| \| \|\|\|\| +\| \| \|+ \|\|\|\|+ \|\|\|\| \| +\|\| \|\| | |
| Sbjct: | 347 | GYVTSTDGSRCIDQRTGMCFSGLVNGRCAQELPGRMTKMQCCCEPGRCWGIGTIPEACPV | 406 |
| Query: | 79 | RGSNEFQQLCAQRLPL--LPGHPGLFPGLLGFGSNGMGPPLGPARLNPHGSDARGIP--- | 133 |
| | | \|\|\| \|+++\|\| \|\|+ +\|\| \| \|\| \| \| \|\| \| \| \|+ \|\| | |
| Sbjct: | 407 | RGSEEYRRLCMDGLPMGGIPGSAGSRPG--GTGGNGFAPSGNGNGYGPGGTGFIPIPGGN | 464 |
| Query: | 134 | --SLGPGNSNIGT----------ATLNQTIDICRHFTNLCLNGRCLPTPSSYRCECNVGY | 181 |
| | | \| \| \| + +\| \|\|\|\|\|\|\|\|+\| \|\|\|\|\|\|\|\|+\|\| \|\|\|\|\|\|\|\|+\|\| | |
| Sbjct: | 465 | GFSPGVGGAGVGAGGQGPIITGLTILNQTIDICKHHANLCLNGRCIPTVSSYRCECNMGY | 524 |
| Query: | 182 | TQDVRGECIDVDECTSSPCHHGDCVNIPGTYHCRCYPGFQATPTRQACVDVDECIVSGGL | 241 |
| | | \|\| \|+\|\|\|\|\|\|\|\|\|+\|\| +\|\|\|\|\| \|\|+\|+\|+\|+ \|\|\| \|\|\|+\|\|\|+\|+\|\|\|\| +\| \| | |
| Sbjct: | 525 | KQDANGDCIDVDECTSNPCTNGDCVNTPGSYYCKCHAGFQRTPTKQACIDIDECIQNGVL | 584 |
| Query: | 242 | CHLGRCVNTEGSFQCVCNAGFELSPDGKNCVDHNECATSTMCVNGVCLNEDGSFSCLCKP | 301 |
| | | \| \|\|\|\|\|++\|\|\|\|\|+\|\|\|\|\|\|\|+ \|\|\|\|\|\|\|\|+\|\| \|+ \|\|+\|\|+\|+\|\|\|\|\|\| \|+\|\|\| | |
| Sbjct: | 585 | CKNGRCVNSDGSFQCICNAGFELTTDGKNCVDHDECTTTNMCLNGMCINEDGSFKCICKP | 644 |
| Query: | 302 | GFLLAPGGHYCMDIDECQTPGICVNGHCTNTEGSFRCQCLGGLAVGTDGRVCVDTHVRST | 361 |
| | | \|\|+\|\|\| \| \|\| \|+\|\|\|\|\|\|\|\|\|+\|\|\|\| \|+\|\|\|\|\|\| \| \|\|\|\|\| \|\|\|\|\|\|\|\|\|+\|\|\| | |
| Sbjct: | 645 | GFVLAPNGRYCTDVDECQTPGICMNGHCINSEGSFRCDCPPGLAVGMDGRVCVDTHMRST | 704 |
| Query: | 362 | CYGAIEKGSCARPFPGTVTKSECCCANPDHGFGEPCQLCPAKNSAEFQALCSSGLGITTD | 421 |
| | | \|\|\| \|+\|\| \| \|\|\|\|\| \|\|\|\|\|\|\|\|\|\|\|\|+\|\|\|\|\|\|\| \|\|\|\|\|\|\|\|\| \|\|\|\|\|+\|\|\| \| | |
| Sbjct: | 705 | CYGGIKKGVCVRPFPGAVTKSECCCANPDYGFGEPCQPCPAKNSAEFHGLCSSGVGITVD | 764 |
| Query: | 422 | GRDINECALDPEVCANGVCENLRGSYRCVCNLGYEAGASGKDCTDVDECALNSLLCDNGW | 481 |
| | | \|\|\|\|\|\|\|\|\|\|\|++\|\|\|\|+\|\|\|\|\|\|\|\|\|\| \|\| \|\|\| \|\|\|++\| \|+\|\|\| +\| \|\|\|\|\|\| | |
| Sbjct: | 765 | GRDINECALDPDICANGICENLRGSYRCNCNSGYEPDASGRNCIDIDECLVNRLLCDNGL | 824 |
| Query: | 482 | CQNSPGSYSCSCPPGFHFWQDTEICKDVDECLSSPCVSGVCRNLAGSYTCKCGPGSRLDP | 541 |
| | | \|+\|+\|\|\|\|\|\|+\|\|\|\|+ \| +\|\| \|+\|++\|\| \|+\|\|\|+\| \|\|\| \|\|+ \|+\| \|\|\|+\| | |
| Sbjct: | 825 | CRNTPGSYSCTCPPGYVFRTETETCEDINECESNPCVNGACRNNLGSFNCECSPGSKLSS | 884 |
| Query: | 542 | SGTFCLDSTKGTCWLKIQESRCEVNLQGASLRSECCATLGAAWGSPCERCEIDPACARGF | 601 |
| | | +\| \|+\|\| \|\|\|\|\|\| \|\|+\|\|\|\|\|\|+ \|\|+\|+\|\|\|\|\|\|\|\|\|\|\|\|\|\|\|\|\|\|\|+\| \|\| \|\| | |
| Sbjct: | 885 | TGLICIDSLKGTCWLNIQDSRCEVNINGATLKSECCATLGAAWGSPCERCELDTACPRGL | 944 |
| Query: | 602 | ARMTGVTCDDVNECESFPGVCPNGRCVNTAGSFRCECPEGLMLDASGRLCVDVRLEPCFL | 661 |
| | | \|\|+ \|\|\|\|+\|\|\|\|\|\| \|\|\|\|\|\|\|\|\|\|\|\|+ \|\|\| \|\|\|\|\|\|\| \|\| +\|\|+\|+\|+\|+\| \|+\| | |
| Sbjct: | 945 | ARIKGVTCEDVNECEVFPGVCPNGRCVNSKGSFHCECPEGLTLDGTGRVCLDIRMEQCYL | 1004 |
| Query: | 662 | RWDEDECGVTLPGKYRMDVCCCSIGAVWGVECEACPDPESLEFASLCPRGLGFASR-DFL | 720 |
| | | +\|\|\|\|\|\| +\|\|\|+\|\|\| \|\|\|++\|\| \|\| \|\|\| \|\| \| + \|+ +\|\|\|\|\| \|\|\|+\| \| \| | |
| Sbjct: | 1005 | KWDEDECIHPVPGKFRMDACCCAVGAAWGTECEECPKPGTKEYETLCPRGAGFANRGDVL | 1064 |
| Query: | 721 | SGRPFYKDVNECKVFPGLCTHGTCRNTVGSFHCACAGGFALDAQERNCTDIDECRISPDL | 780 |
| | | +\|\|\|\|\|\|\|+\|\|\|\| \|\|\|+\|\|+\| \|\|\|\|+\|\|\| \| \| \|\|\|\|\| +\|\|\|\|\|\|\|\|\|\|\|\|\|\|\|\| | |
| Sbjct: | 1065 | TGRPFYKDINECKAFPGMCTYGKCRNTIGSFKCRCNSGFALDMEERNCTDIDECRISPDL | 1124 |
| Query: | 781 | CGQGTCVNTPGSFECECFPGYESGFMLMKNCMDVDECARDPLLCRGGTCTNTDGSYKCQC | 840 |
| | | \|\| \| \|\|\|\|\|\|\|\|\|\|\|\|\| \|\|\|\|\|\|\|+\|\|\|\|\|\|+\| \| \|+\|\|\|\|\|\|\|\|\| \|\|+\|\|++\| \| | |
| Sbjct: | 1125 | CGSGICVNTPGSFECECFEGYESGFMMMKNCMDIDGCERNPLLCRGGTCVNTEGSFQCDC | 1184 |
| Query: | 841 | PPGHELTAKGTACEDIDECSLSDGLCPHGQCVNVIGAFQCSCHAGFQSTPDRQGCVDINE | 900 |
| | | \| \|\|\|\|+ \| \|\|+\|\|\|\|\|\| \|\| +\|+\|\|\|+\|\| +\|\|\|\|+ \|+\|+\|\|\|\|\|\|\| \|\|+\| | |
| Sbjct: | 1185 | PLGHELSPSREDCVDINECSLSDNLCRNGKCVNMIGTYQCSCNPGYQATPDRQGCTDIDE | 1244 |
| Query: | 901 | CRVQNGGCDVHRINTEGSYRCSCGQGYSLMPDGRACADVDECEENPRVCDQGHCTNMPGG | 960 |
| | | \| + \|\|\|\|\| \|+\|\|\|\| \|\|\| +\|\|+\|\|\|\|\|\|+\|\|\|+\|\|\|\| \|\| +\|\| \| \|\|\|+\|\| | |
| Sbjct: | 1245 | CMIMNGGCDTQCTNSEGSYECSCSEGYALMPDGRSCADIDECENNPDICDGGQCTNIPGE | 1304 |
| Query: | 961 | HRCLCYDGFMATPDMRTCVDVDECDLNPHICLHGDCENTKGSFVCHCQLGYMVRKGATGC | 1020 |
| | | +\|\|\|\|\|\|\|\|\|\|+ \|\|+\|\|+\|\|+\|\|\|\|\| +\|\|+ \|+\|\|\|\|\|\|\|\|+\|\|\|\|\|\|\| \|+\|\| \|\|\| | |
| Sbjct: | 1305 | YRCLCYDGFMASMDMKTCIDVNECDLNSNICMFGECENTKGSFICHCQLGYSVKKGTTGC | 1364 |
| Query: | 1021 | SDVDECEVGGHNCDSHASCLNIPGSFSCRCLPGWVGDGFECHDLDECVSQEHRCSPRGDC | 1080 |
| | | +\|\|\|\|\|\|+\| \|\|\|\| \|\|\|\|\|\|\|\|\|\|\| \| \| \|\|+\|+\| +\| \|\|\|\|\| + \|+\|\| \| | |
| Sbjct: | 1365 | TDVDECEIGAHNCDMHASCLNIPGSFKCSCREGWIGNGIKCIDLDECSNGTHQCSINAQC | 1424 |
| Query: | 1081 | LNVPGSYRCTCRQGFAGDCFFCEDRDECAENVDLCDNG 1118 | |
| | | +\| \|\|\|\|\|\| \| +\|\| \|\|\|\| \| \| \|\|\|\|\|\|++\|\|+\|\| | |
| Sbjct: | 1425 | VNTPGSYRCACSEGFTGDGFTCSDVDECAENINLCENG 1462 | |

AMF3 also has high homology to other amino acid sequences, as shown in BLASTP alignment data shown in Table 3F.

TABLE 3F
BLASTP alignment results for AMF3

| | Score | E |
|---|---|---|
| Sequences producing significant alignments: | (bits) | Value |
| FBN2_HUMAN P35556 homo sapiens (human) . fibrillin 2 precurso . . . | 1804 | 0.0 |
| FBN2_MOUSE Q61555 mus musculus (mouse) . fibrillin 2 precurso . . . | 1802 | 0.0 |
| O88840 O88840 mus musculus (mouse) . mutant fibrillin-1. 5/1999 | 1596 | 0.0 |
| FBN1_BOVIN P98133 bos taurus (bovine) . fibrillin 1 precursor . . . | 1594 | 0.0 |
| FBN1_HUMAN P35555 homo sapiens (human) . fibrillin 1 precurso . . . | 1591 | 0.0 |
| FBN1_MOUSE 061554 mus musculus (mouse) . fibrillin 1 precurso . . . | 1590 | 0.0 |
| Q60784 Q60784 mus musculus (mouse) . fibrillin-1 (fragment) . . . . | 1108 | 0.0 |
| P87363 P87363 gallus gallus (chicken) . fibrillin-1 (fragment . . . | 713 | 0.0 |
| Q60789 Q60789 mus musculus (mouse) . fibrillin-2 (fragment) . . . . | 534 | e−150 |

The presence of identifiable domains in AMF3, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMFX RNA was derived using tissue sources including, but not limited to, proprietary database sources, public EST sources, literature sources, and/or RACE sources, as described in the Examples. AMF3 is expressed in at least the following tissues: colon and gastric cancers. Highest expression is lung cancer cell lines and this correlates with expression in fetal lung, indicating an oncofetal phenotype.

The nucleic acids and proteins of AMF3 are useful in potential therapeutic applications implicated in various AMF-related pathologies and/or disorders. For example, a cDNA encoding the fibrillin-like protein may be useful in gene therapy, and the fibrillin-like protein may be useful when administered to a subject in need thereof. The novel nucleic acid encoding AMF3 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMF3 nucleic acids and proteins are useful in potential diagnostic and therapeutic applications implicated in various diseases and disorders described below and/or other pathologies. For example, the compositions of the present invention will have efficacy for treatment of patients suffering from: Marfan syndrome, congenital contractural arachnodactyly, Marfan-like habitus, familial adenomatous polyposis and other diseases, disorders and conditions of the like. By way of nonlimiting example, the compositions of the present invention will have efficacy for treatment of patients suffering from Marfan syndrome, congenital contractural arachnodactyly, Marfan-like habitus, familial adenomatous polyposis. Additional AMF3-related diseases and disorders are mentioned throughout the Specification.

Further, the protein similarity information, expression pattern, and map location for AMF3 suggests that AMF3 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF3 substances for use in therapeutic or diagnostic methods. These antibodies may be generated according to methods known in the art, using prediction from hydrophobicity charts, as described in the “Anti-AMFX Antibodies” section below. In various embodiments, contemplated AMF3 epitopes are hydrophilic regions of the AMF3 polypeptide as predicted by software programs well known in the art that generate hydrophobicity or hydrophilicity plots.

AMF-4 (Also Referred to as Acc. No. 27486474)

Novel AMF4 is a plasminogen-like protein. The AMF4 clone is alternatively referred to herein as Acc. No. 27486474. The AMF4 nucleic acid of 439 nucleotides is shown in Table 4A. The AMF4 open reading frame (“ORF”) begins at positions 2-5. The AMF4 ORF terminates at a TAA codon at nucleotides 93-95. As shown in Table 4A, putative untranslated regions 3′ to the stop codon are underlined, and the stop codon is in bold letters. AMF4 does not begin at an ATG start site, so it is most likely a C-terminal coding fragment. It is contemplated that the AMF4 ORF extends in the 5′ direction of the nucleic acid (SEQ ID NO:7) and the N-terminal direction of the polypeptide (SEQ ID NO:8).

TABLE 4A
AMF4 nucleic acid (SEQ ID NO:7)

T CAC GGG AAT AAG CCT GGG CCC GTC CCT TTG ATT TCC AAC AAG ATC
TGC AAC CAC AGG GAC GTG TAC GGT GGC ATC ATC TCC CCC TCC ATG
CTC TGC GCG GGC TAC CTG ACG GGT GGC GTG GAC AGC TGC CAG GGG
GAC AGC GGG GGG CCC CTG GTG TGT CAA GAG AGG AGG CTG TGG AAG
TTA GTG GGA GCG ACC AGC TTT GGC ATC GGC TGC GCA GAG GTG AAC
AAG CCT GGG GTG TAC ACC GTG TCA CCT CCT TCC TGG ACT GGA TCC
ACG AGC AGA TGG AGA GAG ACC TAA AAA CCT GAA GAG GAA GGG GAT
AAG TAG CCA CCT GAG TTC CTG AGG TGA TGA AGA CAG CCC GAT CCT
CCC CTG GAC TCC CGT GTA GGA ACC TGC ACA CGA GCA GAC ACC CTT
GGA GCT CTG AGT TCC GGC ACC AGT AGC AGG CCC

The encoded AMF4 polypeptide (SEQ ID NO:8) is shown using the one-letter amino acid code in Table 4B.

TABLE 4A
AMF4 polypeptide (SEQ ID NO:8)

HGNKPGPVPLISNKICNHRDVYGGIISPSMLCAGYLTGGVDSCQGDSGGPLVCQERRLWKLV
GATSFGIGCAEVNKPGVYTVSPPSWTGSTSRWRET

In an analysis of public nucleic acid sequence databases, it was found, for example, that the AMF4 nucleic acid sequence has 418 of 420 bases (99%) identical to a serine protease (GenBank Acc. No. AB038159) (SEQ ID NO:69) shown in Table 4C. In all BLAST alignments herein, the “E-value” or “Expect” value is a numeric indication of the probability that the aligned sequences could have achieved their similarity to the BLAST query sequence by chance alone, within the database that was searched. For example, as shown in Table 4C, the probability that the subject (“Sbjct”) retrieved from the AMF4 BLAST analysis, in this case the serine protease gene/protein, matched the Query AMF4 sequence purely by chance is zero, E value 0.0.

TABLE 4C
BLASTN of AMF4 against AB038159 (SEQ ID NO:69)

>AB038159 H. sapiens TMPRSS3c mRNA for serine protease, complete cds. 1/2001
Length = 2135 Strand = Plus/Plus
Score = 809 bits (408), Expect = 0.0
Identities = 418/420 (99%), Gaps = 1/420 (0%)

| | | | |
|---|---|---|---|
| Query: | 21 | ccgtccctttgatttccaacaagatctgcaaccacagggacgtgtacggtggcatcatct | 80 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 950 | ccgtccctttgatttccaacaagatctgcaaccacagggacgtgtacggtggcatcatct | 1009 |
| Query: | 81 | ccccctccatgctctgcgcgggctacctgacgggtggcgtggacagctgccagggggaca | 140 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1010 | ccccctccatgctctgcgcgggctacctgacgggtggcgtggacagctgccagggggaca | 1069 |
| Query: | 141 | gcggggggcccctggtgtgtcaagagaggaggctgtggaagttagtgggagcgaccagct | 200 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1070 | gcggggggcccctggtgtgtcaagagaggaggctgtggaagttagtgggagcgaccagct | 1129 |
| Query: | 201 | ttggcatcggctgcgcagaggtgaacaagcctggggtgtaca-ccgtgtcacctccttcc | 259 |
| | | \|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| | |
| Sbjct: | 1130 | ttggcatcggctgcgcagaggtgaacaagcctggggtgtacacccgtgtcacctccttcc | 1189 |
| Query: | 260 | tggactggatccacgagcagatggagagagacctaaaaacctgaagaggaaggggataag | 319 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\| | |
| Sbjct: | 1190 | tggactggatccacgagcagatggagagagacctaaaaacctgaagaggaaggggacaag | 1249 |
| Query: | 320 | tagccacctgagttcctgaggtgatgaagacagcccgatcctcccctggactcccgtgta | 379 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1250 | tagccacctgagttcctgaggtgatgaagacagcccgatcctcccctggactcccgtgta | 1309 |
| Query: | 380 | ggaacctgcacacgagcagacacccttggagctctgagttccggcaccagtagcaggccc | 439 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1310 | ggaacctgcacacgagcagacacccttggagctctgagttccggcaccagtagcaggccc | 1369 |

Additional BLASTN information for related nucleic acid sequences is shown in Table 4D.

TABLE 4D
BLASTN analysis results for AMF4

| | Score | E |
|---|---|---|
| Sequences producing significant alignments: | (bits) | Value |
| AB038159 AB038159 Homo sapiens TMPRSS3c mRNA for serine prot . . . . | 809 | 0.0 |
| AB038158 AB038158 Homo sapiens TMPRSS3b mRNA for serine prot . . . | 809 | 0.0 |
| AB038157 AB038157 Homo sapiens TMPRSS3a mRNA for serine prot . . . | 809 | 0.0 |
| AF201380 AF201380 Homo sapiens serine protease TADGl2 mRNA, . . . | 753 | 0.0 |
| AP001746 AP001746 Homo sapiens genomic DNA, chromosome 21q, . . . | 301 | 2e−79 |
| AP001823 AP001523 Homo sapiens genomic DNA, chromosome 21, c . . . . | 301 | 2e−79 |
| AC015555 AC015555 Homo sapiens chromosome 21 clone RP11-113F . . . | 301 | 2e−79 |

A BLASTP search was performed against public protein databases. The results from this comparison are shown in Table 4E.

TABLE 4E
BLASTP analysis results for AMF4

| | Score | E |
|---|---|---|
| Sequences producing significant alignments: | (bits) | Value |
| PLMN_PIG P06867 sus scrofa (pig) . plasminogen (ec 3.4.21.7) . . . . | 102 | 6e−22 |
| PLMN_BOVIN P06868 bos taurus (bovine) . plasminogen precursor . . . | 101 | 2e−21 |
| HEPS_MOUSE O35453 mus musculus (mouse) . serine protease heps . . . | 98 | 2e−20 |
| PLMN_HORSE P80010 equus caballus (horse) . plasminogen (ec 3 . . . . | 97 | 3e−20 |
| PLMN_MACMU P12845 macaca mulatta (rhesus macaque) . plasminog . . . | 96 | 5e−20 |
| HEPS_RAT Q05511 rattus norvegicus (rat). serine protease hep . . . | 96 | 5e−20 |
| REPS_HUMAN P05981 homo sapiens (human) . serine protease heps . . . | 96 | 5e−20 |
| PLMN_HUMAN P00747 homo sapiens (human) . plasminogen precurso . . . | 96 | 6e−20 |
| Q15146 Q15146 homo sapiens (human) . plasminogen precursor . 1 . . . | 96 | 6e−20 |
| O46507 O46507 papio hamadryas (hamadryas baboon) . plasminoge . . . | 96 | 8e−20 |

For example, as shown in Table 4F, the AMF4 protein has 48 of 81 amino acid residues (59%) identical to, and 60 of 81 residues (73%) positive with, the 790 amino acid residue long plasminogen from pig (Acc. No. P06867) (SEQ ID NO:70).

TABLE 4F
BLASTP of AMF4 against P06867 (SEQ ID NO:70)

PLM_PIG P06867 sus scrofa (pig). plasminogen (ec 3.4.21.7). 10/1996
Length = 790
Score = 102 bits (252), Expect = 6e-22
Identities = 48/81 (59%), Positives = 60/81 (73%), Gaps = 1/81 (1%)

| | | | |
|---|---|---|---|
| Query: | 4 | KPGPVPLISNKICNHRDVYGGIISPSMLCAGYLRGGVDSCQGDSGGPLVCQERRLWKLVG | 63 |
| | | \| +\|+\| \|\|+\|\| + \|\| +\|\|+ \|\|\|\|+\| \|\|+\|\|\|\|\|\|\|\|\|\|\|\|\| \|+ + \| \| | |
| Sbjct: | 697 | KEARLPVIENKVCNRYEYLGGKVSPNELCAGHLAGGIDSCQGDSGGPLVCFEKDKYILQG | 756 |
| Query: | 64 | ARSFGIGCAEVNKPGVY-RVS 83 | |
| | | \|+\|+\|\|\| \|\|\|\|\|\| \|\|\| | |
| Sbjct: | 757 | VTSWGLGCALPNKPGVYVRVS 777 | |

In addition, as shown in Table 4G, the AMF4 protein has 47 of 82 amino acid residues (57%) identical to, and 58 of 82 residues (70%) positive with, the 812 amino acid residue long bovine plasminogen precursor (Acc. No. P06868) (SEQ ID NO:71).

TABLE 4G
BLASTP of AMF4 against P06868 (SEQ ID NO:71)

PLMN_BOVIN P06868 bos taurus plasminogen precursor (ec 3.4.21.7) 11/1997
Length = 812
Score = 101 bits (248), Expect = 2e-21
Identities = 47/82 (57%), Positives = 58/82 (70%), Gaps = 1/82 (1%)

| | | | |
|---|---|---|---|
| Query: | 4 | KPGPVPLISNKICNHRDVYGGIISPSMLCAGYLRGGVDSCQGDSGGPLVCQERRLWKLVG | 63 |
| | | \| +\|+\| \|\|+\|\| + \| + \|+ \|\|\|\|+\| \|\| \|\|\|\|\|\|\|\|\|\|\|\|\| \|+ + \| \| | |
| Sbjct: | 719 | KEAHLPVIENKVCNRNEYLDGRVKPTELCAGHLIGGTDSCQGDSGGPLVCFEKDKYILQG | 778 |
| Query: | 64 | ARSFGIGCAEVNKPGVY-RVSP 84 | |
| | | \|+\|+\|\|\| \|\|\|\|\|\| \|\|\|\| | |
| Sbjct: | 779 | VTSWGLGCARPNKPGVYVRVSP 800 | |

The presence of identifiable domains in AMF4, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections. For this DOMAIN sequence alignments, fully conserved single residues are indicated by black shading “strong” semi-conserved residues are indicated by grey. The “strong” group of conserved amino acid residues may be any one of the following groups of amino acids: STA, NEQK, NHQK, NDEQ, QHRK, MILV, MILF, HY, FYW. AMF4 shows good homology with the consensus sequence of the trypsin-like serine protease domain (Smart|Tryp SPc, E=2e-21) and the trypsin domain (Pfam00089, E=2e-14). The alignment with the trypsin-like serine protease domain (SEQ ID NO:72)(labeled “Consensus”) is shown in Table 4H.

TABLE 4H
DOMAIN ANALYSIS FOR AMF4 - ALIGNMENT WITH TRYPSIN-LIKE SERINE PROTEASE DOMAIN (SEQ ID NO:72)

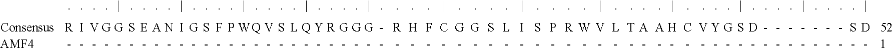

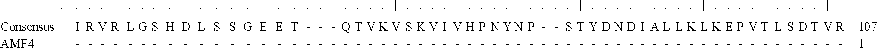

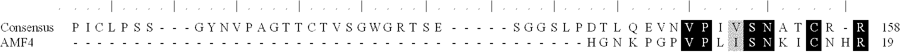

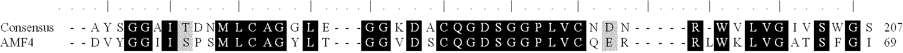

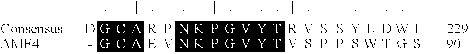

The trypsin-like serine protease domain is present in a large family of proteins, including many that are synthesized as inactive precursor zymogens that are cleaved during limited proteolysis to generate their active forms.

AMF4 has similarity to plasminogens. Plasmin dissolves the fibrin of blood clots and acts as a proteolytic factor in a variety of other processes including embryonic development, tissue remodeling, tumor invasion, and inflammation; in ovulation it weakens the walls of the graafian follicle. It activates the urokinase-type plasminogen activator, collagenases and several complement zymogens, such as c I and c5. it cleaves fibrin, fibronectin, thrombospondin, laminin and von Willebrand factor.

Plasminogen is the zymogen in the circulating blood from which plasmin is formed. Plasminogen is a single-chain glycoprotein with 790 amino acid residues. Activation to the active form, plasmin, by urokinase (Online Mendelian Inheritance in Man (“OMIM”) Acc. No. 191840) involves cleavage at the Arg-Val bond between residues 560 and 561, resulting in the formation of the 2-chain plasmin molecule held together by 2 disulfide linkages. The heavier chain contains about 411 residues and the lighter chain about 233. The main function of plasmin is the digestion of fibrin in blood clots. Plasmin is a proteolytic enzyme with a specificity similar to that of trypsin. Like trypsin, plasmin belongs to the family of serine proteinases, in which the active site catalytic triad, His-57, Asp-102, and Ser-195 (chymotrypsin numbering), is situated in the light chain.

The plasminogen activation system is one pathway that has been consistently implicated in cancer. Its relevance to cancer extends from being responsible for many of the hemorrhagic episodes that occur in cancer patients to being fundamental to many, if not all of the molecular mechanisms that define tumor progression. Extravasation and intravasation of solid malignant tumors is controlled by attachment of tumor cells to components of the basement membrane and the extracellular matrix, by local proteolysis and tumor cell migration. Strong clinical and experimental evidence has accumulated that the tumor-associated serine protease plasmin, its activator uPA (urokinase-type plasminogen activator), the receptor uPA-R (CD87), and the inhibitors PAI-I and PAI-2 are linked to cancer invasion and metastasis. In cancer, increase of uPA, uPA-R, and/or PAI-1 is associated with tumor progression and with shortened disease-free and/or overall survival in patients afflicted with malignant solid tumors. uPA and/or its inhibitor PAI-1 appear to be one of the strongest prognostic markers so far described. Strong prognostic value to predict disease recurrence and overall survival has been documented for patients with cancer of the breast, ovary, cervix, endometrium, stomach, colon, lung, bladder, kidney, brain, and soft-tissue. Due to the strong correlation between elevated uPA and/or PAI-1 values in primary cancer tissues and the tumor invasion/metastasis capacity of cancer cells, proteolytic factors have been selected as targets for therapy.

A novel angiogenesis inhibitor that mediated the suppression of metastases from a Lewis lung carcinoma was isolated and designated the inhibitor angiostatin. See, e.g., O'Reilly et al. 1994 Cell 79: 315-328. Angiostatin is a 38-kD internal fragment of plasminogen containing at least 3 of the kringles of plasminogen. Recombinant fragments of angiostatin show inhibitory activity in vitro. See, e.g., Cao et al. 1996 J. Clin. Invest. 101: 1055-1063. Angiostatin is produced by the proteolytic cleavage of plasminogen by a serine protease produced by several human prostate carcinoma cell lines. See, e.g., Gately et al. 1996 Cancer Res. 56: 4887-4890. A shift of balance of tumor angiogenesis by gene transfer of a cDNA coding for mouse angiostatin into murine T241 fibrosarcoma cells suppresses primary and metastatic tumor growth in vivo. See, e.g., Cao et al. 1998 J. Clin. Invest. 101: 1055-1063. Implementation of stable clones expressing mouse angiostatin in C57B16/J mice inhibited primary tumor growth by an average of 77%. After removal of primary tumors, the pulmonary micrometastases in approximately 70% of mice remained in a microscopic dormant and avascular state for 2 to 5 months. The tumor cells in the dormant micrometastases exhibited a high rate of apoptosis balanced by a high proliferation rate. These studies showed the diminished growth of lung metastases after removal of the primary tumor, suggesting that metastases are self-inhibitory by halting angiogenesis. The data may also provide a novel approach for cancer therapy by anti-angiogenic gene therapy with a specific angiogenesis inhibitor. The angiostatin-induced long-term dormancy of lung metastases was equivalent to 14 to 15 human years (when 1 mouse day is equivalent to approximately 35 human days).

Overexpression of AMF4 in concert with a plasminogen activator such as uPA (urokinase) could potentially stimulate tumor cell invasion and migration. Alternatively, AMF4 could serve as a substrate for an unidentified serine protease akin to the protease that cleaves plasminogen to angiostatin. In this manner, tumor cells might limit the production of this important anti-angiogenic factor.

Therapeutic targeting of AMF4 is anticipated to limit or block the extent of tumor cell invasion/motility and metastasis. Potentially therapeutic targeting of AMF4 might shift the balance in favor of the production of angiostatin or a similar molecule with anti-angiogenic activity.

Expression information for AMFX RNA was derived using tissue sources including, but not limited to, proprietary database sources, public EST sources, literature sources, and/or RACE sources, as described in the Examples.

The nucleic acids and proteins of AMF4 are useful in potential therapeutic applications implicated in various AMF-related pathologies and/or disorders. For example, a cDNA encoding the trypsin-like serine protease protein may be useful in gene therapy, and the trypsin-like serine protease protein may be useful when administered to a subject in need thereof. The novel nucleic acid encoding AMF4 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from: cancer, blood clotting disorders and other diseases, disorders and conditions of the like. By way of nonlimiting example, the compositions of the present invention will have efficacy for treatment of patients suffering from cancer, blood clotting disorders. Additional AMF-related diseases and disorders are mentioned throughout the Specification.

Further, the protein similarity information, expression pattern, and map location for AMF4 suggests that AMF4 may have important structural and/or physiological functions characteristic of the trypsin-like serine protease family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF4 substances for use in therapeutic or diagnostic methods. These antibodies may be generated according to methods known in the art, using prediction from hydrophobicity charts, as described in the “Anti-AMFX Antibodies” section below. In various embodiments, contemplated AMF4 epitopes are hydrophilic regions of the AMF4 polypeptide as predicted by software programs well known in the art that generate hydrophobicity or hydrophilicity plots.

AMF-5 (Also Referred to as Ace. No. 29691387)

Novel AMF5 is an organic anion transporting peptide-like protein (“OTAP”) protein. The AMF5 clone is alternatively referred to herein as Acc. No. 29691387. The AMF5 nucleic acid of 2646 nucleotides is shown in Table 5A. The AMF5 open reading frame (“ORF”) begins at nucleotides 3-5. AMF5 appears to be an internal fragment, so it is contemplated that the ORF could extend beyond the N- and C-termini depicted in Tables 5A and 5B. As shown in Table 5A, the first coding triplet is in bold letters.

TABLE 5A
AMF5 nucleotide sequence (SEQ ID NO:9).

TGTCATTGTCCTTTTACCTATTATATTTTTTCATACTCTGTGAAAACAAATCAGTTGCCGGACTAACCATGACCTA
TGATGGAAATAATCCAGTGACATCTCATAGAGATGTGCCACTTTCTTATTGCAACTCAGACTGCAATTGTGATGAA
AGTCAGTGGGAACCAGTCTGTGGGAACAATGGAATAACTTACCTGTCACCTTGTCTAGCAGGATGCAAATCCTCAA
GTGGTATTAAAAAGCATACAGTGTTTTATAACTGTAGTTGTGTGGAAGTAACTGGTCTCCAGAACAGAAATTACTC
AGCGCACTTGGGTGAATGCCCAAGAGATAATACTTGTACAAGGAAATTTTTCATCTATGTTGCAATTCAAGTCATA
AACTCTTTGTTCTCTGCAACAGGAGGTACC

The encoded AMF5 protein (SEQ ID NO:10) is a 136 amino acid protein shown in Table 5B.

### Table 5B. AMF5 Amino Acid Sequence (SEQ ID NO:10)

SLSFYLLYFFILCENKSVAGLTMTYDGNNPVTSHRDVPLSYCNSDCNCDESQWEPVCGNNGITYLSPCLAGCKSSS GIKKHTVFYNCSCVEVTGLQNRNYSAHLGECPRDNTCTRKFFIYVAIQVINSLFSATGGT

In an analysis of public nucleic acid sequence databases, it was found, for example, that the AMF5 nucleic acid sequence has 363 of 374 bases (97%) identical to a Homo sapiens mRNA for organic anion transporter 8 (SLC21A8 gene) (GenBank Acc. No. AJ251506) (SEQ ID NO:73) shown in Table 5C.

TABLE 5C
BLASTN of AMF5 against OAT-8 mRNA (SEQ ID NO:73)

>HSA251506 AJ251506 Homo sapiens mRNA for organic anion transporter 8
(SLC21A8 gene). 7/2000 Length = 2646; Strand = Plus/Plus
Score = 654 bits (330), Expect = 0.0
Identities = 363/374 (97%)

| | | | |
|---|---|---|---|
| Query: | 37 | tctgtgaaaacaaatcagttgccggactaaccatgacctatgatggaaataatccagtga | 96 |
| | | \|\|\|\| \|\|\|\| \|\|\|\|\|\|\|\|\|\|\|\|\|\|\| \|\|\|\|\|\| \|\|\|\|\|\|\|\|\|\|\|\|\|\|\|\|\|\|\|\| \|\|\|\|\| | |
| Sbjct: | 1330 | tctgcgaaagcaaatcagttgccggcctaaccttgacctatgatggaaataattcagtgg | 1389 |
| Query: | 97 | catctcatagagatgtgccactttcttattgcaactcagactgcaattgtgatgaaagtc | 156 |
| | | \|\|\|\|\|\|\|\| \|\|\|\|\|\| \|\|\|\|\|\|\|\|\|\|\|\|\|\|\|\|\|\|\|\|\|\|\| \|\|\|\|\|\|\|\|\|\|\|\|\|\|\|\|\|\|\| | |
| Sbjct: | 1390 | catctcatgtagatgtaccactttcttattgcaactcagagtgcaattgtgatgaaagtc | 1449 |
| Query: | 157 | agtgggaaccagtctgtgggaacaatggaataacttacctgtcaccttgtctagcaggat | 216 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1450 | agtgggaaccagtctgtgggaacaatggaataacttacctgtcaccttgtctagcaggat | 1509 |
| Query: | 217 | gcaaatcctcaagtggtattaaaaagcatacagtgttttataactgtagttgtgtggaag | 276 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1510 | gcaaatcctcaagtggtattaaaaagcatacagtgttttataactgtagttgtgtggaag | 1569 |
| Query: | 277 | taactggtctccagaacagaaattactcagcgcacttgggtgaatgcccaagagataata | 336 |
| | | \|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\| | |
| Sbjct: | 1570 | taactggtctccagaacagaaattactcagcacacttgggtgaatgcccaagagataata | 1629 |
| Query: | 337 | cttgtacaaggaaatttttcatctatgttgcaattcaagtcataaactctttgttctctg | 396 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1630 | cttgtacaaggaaatttttcatctatgttgcaattcaagtcataaactctttgttctctg | 1689 |
| Query: | 397 | caacaggaggtacc | 410 |
| | | \|\|\|\|\|\|\|\|\|\|\|\|\|\| | |
| Sbjct: | 1690 | caacaggaggtacc | 1703 |

In addition, the AMF5 nucleic acid sequence has high homology to other nucleic acid sequences whose BLASTN alignment data is shown in Table 5D.

TABLE 5D
BLASTN alignment results for AMF5

| | Score | E |
|---|---|---|
| Sequences producing significant alignments: | (bits) | Value |
| HSA251506 AJ25l506 Homo sapiens mRNA for organic anion trans . . . | 654 | 0.0 |
| AF187815 AF187815 Homo sapiens liver-specific organic anion . . . | 654 | 0.0 |
| AF205071 AF205071 Homo sapiens organic anion transport polyp . . . | 557 | e−156 |
| AF060500 AF060500 Homo sapiens liver specific transporter mR . . . | 557 | e−156 |
| AB026257 AB026257 Homo sapiens mRNA for organic anion transp . . . | 557 | e−156 |
| HSA132573 AJ132573 Homo sapiens mRNA for organic anion trans . . . | 549 | e−154 |

A BLASTP search was performed against public protein databases. As shown in Table 5E, the AMF5 protein has 119 of 136 amino acid residues (87%) identical to, and 125 of 136 residues (91%) positive with, the 691 amino acid residue long Homo sapiens (human). liver-specific organic anion transporter (organic anion transport polypeptide 2) (oatp 2) (Acc. No.) (SEQ ID NO:74).

TABLE 5E
BLASTP of AMF5a against OATP (SEQ ID NO:74)

>OAT6_HUMAN Q9y616 homo sapiens (human). liver-specific organic anion
transporter (organic anion transport polypeptide 2) (oatp 2). 10/2000 Length = 691
Score = 265 bits (670), Expect = 9e-71
Identities = 119/136 (87%), Positives = 125/136 (91%)

| | | | |
|---|---|---|---|
| Query: | 1 | SLSFYLLYFFILCENKSVAGLRMRYDGNNPVTSHRDVPLEYCNSDCNCDESQWEPVCGNN | 60 |
| | | \|\|\|\|\|\|\|\|\|\|\|\|\|\|\|\|\|\|\|\|\| \| \|\|\|\|\|\|\|\|\|\|\|\|\|\|\| \|\|\|\|\|\|\|\|\|\|\|\|\|\|\|\|\|\|\|\| | |
| Sbjct: | 418 | SLSFYLLYFFILCENKSVAGLTMTYDGNNPVTSHRDVPLSYCNSDCNCDESQWEPVCGNN | 477 |
| Query: | 61 | GITYLSPCLAGCKSSSGIKKHTVFYNCSCVEVTGLQNRNYSAHLGECPRDNTCTRKFFIY | 120 |
| | | \|\|\|\|+\|\|\|\|\|\|\|\|\|\|\|\| \|\| \|\|\|\|\|\|\|+\|\|\|\|\|\|\|\|\|\|\|\|\|\|\|\|\|\|\|\|+ \|\|\|\|\|+ + | |
| Sbjct: | 478 | GITYISPCLAGCKSSSGNKKPIVFYNCSCLEVTGLQNRNYSAHLGECPRDDACTRKFYFF | 537 |
| Query: | 121 | VAIQVINSLFSATGGT | 136 |
| | | \|\|\|\|\|+\| \|\|\| \|\|\| | |
| Sbjct: | 538 | VAIQVLNLFFSALGGT | 553 |

The amino acid sequence of AMF5 also has high homology to the amino acid sequences shown in BLASTP alignment data in Table 5F

TABLE 5F
BLASTP alignment results for AMF5

| | Score | E |
|---|---|---|
| Sequences producing significant alignments: | (bits) | Value |
| OAT6_HUMAN Q9y616 homo sapiens (human) . liver-specific organ . . . | 285 | 9e−71 |
| OAT3_RAT O88397 rattus norvegicus (rat) . sodium-independent . . . | 108 | 2e−23 |
| O88397 O88397 rattus norvegicus (rat) . organic anion transpo . . . | 108 | 2e−23 |
| OATP_HUMAN P46721 homo sapiens (human) . sodium-independent o . . . | 106 | 9e−23 |
| OAT2_RAT O35913 rattus norvegicus (rat) . sodium-independent . . . | 102 | 1e−21 |
| OATP_RAT P46720 rattus norvegicus (rat) . sodium-independent . . . | 99 | 8e−21 |
| OATK_RAT P70502 rattus norvegicus (rat) . sodium-independent . . . | 98 | 2e−20 |
| P70502 P70502 rattus norvegicus (rat) . oat-k1. 1/1999 | 98 | 2e−20 |

The presence of identifiable domains in AMF5, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMFX RNA was derived using tissue sources including, but not limited to, proprietary database sources, public EST sources, literature sources, and/or RACE sources, as described in the Examples. AMF5 is expressed in at least the following tissues: liver, brain, lung, kidney, and testis; additional transcripts were also observed. The authors stated that the extra-hepatic expression of OATP suggests a general role for OATP in trans-epithelial organic anion transport.

The nucleic acids and proteins of AMF5 are useful in potential therapeutic applications implicated in various AMF-related pathologies and/or disorders. For example, a cDNA encoding the organic anion transporting peptide-like protein may be useful in gene therapy, and the organic anion transporting peptide-like protein may be useful when administered to a subject in need thereof. The novel nucleic acid encoding AMF5 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from: colon adenocarcinomas, small cell lung cancers, ovarian cancers, prostate cancers and gliomas, and other diseases, disorders and conditions of the like. By way of nonlimiting example, the compositions of the present invention will have efficacy for treatment of patients suffering from colon adenocarcinomas, small cell lung cancers, ovarian cancers, prostate cancers and gliomas. Additional AMF-related diseases and disorders are mentioned throughout the Specification.

Further, the protein similarity information, expression pattern, and map location for AMF5 suggests that AMF5 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF5 substances for use in therapeutic or diagnostic methods. These antibodies may be generated according to methods known in the art, using prediction from hydrophobicity charts, as described in the “Anti-AMFX Antibodies” section below. In various embodiments, contemplated AMF5 epitopes are hydrophilic regions of the AMF5 polypeptide as predicted by software programs well known in the art that generate hydrophobicity or hydrophilicity plots.

AMF-6 (Also Referred to as Acc. No. 38905521)

Novel AMF6 is MEGF protein-related. The AMF6 clone is alternatively referred to herein as Acc. No. 38905521. The AMF6 nucleic acid (SEQ ID NO:11) of 332 nucleotides is shown in Table 6A. The AMF6 open reading frame (“ORF”) begins at nucleotides 3-5. The AMF6 ORF terminates at nucleotides 318-320. AMF5 appears to be an internal fragment so it is contemplated that the ORF could extend beyond the N- and C-termini. As shown in Table 6A, putative untranslated regions 5′ to the start codon and 3′ to the stop codon are underlined, and the start and stop codons are in bold letters.

TABLE 6A
AMF6 nucleotide sequence (SEQ ID NO:11).

TGGCAGCCCTGGAGGAGCCGATGGTGGACCTGGACGGCGAGCTGCCTTTCGTGCGGCCCCTGCCCCACATTGCCGT
GCTCCAGGACGAGCTGCCGCAACTCTTCCAGGATGACGACGTCGGGGCCGATGAGGAAGAGGCAGAGTTGCGGGGC
GAACACACGCTCACAGAGAAGTTTGTCTGCCTGGATGACTCCTTTGGCCATGACTGCAGCTTGACCTGTGATGACT
GCAGGAACGGAGGGACCTGCCTCCTGGGCCTGGATGGCTGTGATTGCCCCGAGGGGTGGACTGGGGTTATTTGCAA
TGAGATTTGTCCTCCGGA

The encoded AMF6 protein (SEQ ID NO:12) is a 106 amino acid protein shown in Table 6B.

### Table 6B. AMF6 amino acid sequence (SEQ ID NO:12)

AALEEPMVDLDGELPFVRPLPHIAV_{L}QDELPQLFQDDDVGADEEEAELRGEHTLTEKFVCLDDSFGHDCSLTCDDC RNGGTCLLGLDGCDCPEGWTGVICNEICPP

In an analysis of public nucleic acid sequence databases, it was found, for example, that the AMF6 nucleic acid sequence has one fragment 154 of 179 bases (86%) identical and a second fragment 79 of 91 bases (86%) identical to Rattus norvegicus mRNA for MEGF6, complete cds (GenBank Acc. No. ABO 1532) (SEQ ID NOs:75 and 76) shown in Table 6C.

TABLE 6C
BLASTN of AMF6 against MEGF6 mRNA (SEQ ID NO:75 and 76)

>AB011532 AB011532 Rattus norvegicus mRNA for MEGF6, complete cds. 8/1998
Length = 5523
Score = 157 bits (79), Expect = 4e-36
Identities = 154/179 (86%)
Sbjct: residues 1738 to 1916 (SEQ ID NO:75); Strand = Plus/Plus

| | | | |
|---|---|---|---|
| Query: | 141 | gagttgcggggcgaacacacgctcacagagaagtttgtctgcctggatgactcctttggc | 200 |
| | | \|\|\|\|\|\|\|\| \|\| \|\|\|\|\|\|\|\|\|\|\|\|\|\| \|\|\|\|\|\|\|\|\|\|\|\|\|\|\| \|\|\|\|\| \|\|\|\|\|\|\| \|\| | |
| Sbjct: | 1738 | gagttgcgtggagaacacacgctcactgagaagtttgtctgcttggatcactccttcggg | 1797 |
| Query: | 201 | catgactgcagcttgacctgtgatgactgcaggaacggagggacctgcctcctgggcctg | 260 |
| | | \|\|\|\|\|\|\|\|\|\|\|\| \| \|\|\|\|\| \|\|\|\|\|\|\|\|\|\|\|\|\|\| \|\| \|\|\|\|\| \|\|\| \|\|\| \|\|\|\|\| \| | |
| Sbjct: | 1798 | catgactgcagcctaacctgcgatgactgcaggaatggggggacttgcttcccgggccag | 1857 |
| Query: | 261 | gatggctgtgattgccccgaggggtggactggggttatttgcaatgagatttgtcctcc | 319 |
| | | \|\| \|\|\|\|\|\|\|\| \|\|\|\|\| \|\|\|\|\| \|\|\|\|\|\|\|\| \| \|\| \|\|\|\|\|\|\|\|\|\| \|\|\|\|\|\|\|\|\| | |
| Sbjct: | 1858 | gacggctgtgactgcccagagggctggactggaatcatctgcaatgagacttgtcctcc | 1916 |

Score = 85.7 bits (43), Expect = 1e-14
Identities = 79/91 (86%)
Sbjct: residues 1616 to 1706 (SEQ ID NO:76); Strand Plus/Plus

| | | | |
|---|---|---|---|
| Query: | 22 | tggtggacctggacggcgagctgcctttcgtgcggcccctgccccacattgccgtgctcc | 81 |
| | | \|\|\|\|\|\|\|\|\|\|\|\|\| \|\|\| \|\|\|\|\|\| \|\| \|\|\|\|\|\|\|\|\|\|\|\|\|\|\|\|\|\|\|\|\|\|\| \|\|\|\|\| | |
| Sbjct: | 1616 | tggtggacctggatggccggctgccctttgtgcggcccctgccccacattgcggtgctga | 1675 |
| Query: | 82 | aggacgagctgccgcaactcttccaggatga | 112 |
| | | \|\|\| \|\|\|\|\|\|\|\| \| \|\|\|\|\|\|\|\|\|\|\|\|\|\|\| | |
| Sbjct: | 1676 | gggatgagctgccccgactcttccaggatga | 1706 |

A BLASTP search was performed against public protein databases. As shown in Table 6D, the AMF6 protein has 89 of 107 amino acid residues (83%) identical to, and 95 of 107 residues (88%) positive with, the 1574 amino acid residue long Rattus norvegicus (rat). megf6 (Acc. No. 088281) (SEQ ID NO.77).

TABLE 6D
BLASTP of AMF6a against MEGF6 (SEQ ID NO:77)

>O88281 O88281 rattus norvegicus (rat). megf6. 5/1999 Length = 1574
Score = 194 bits (489), Expect = 1e-49
Identities = 89/107 (83%), Positives = 95/107 (88%), Gaps = 3/107 (2%)

| | | | |
|---|---|---|---|
| Query: | 2 | ALEEPMVDLDGELPFVRPLPHIAVLQDELPQLFQDDDVGADEEEA--ELRGEHTLTEKFV | 59 |
| | | +\|\|\| +\|\|\|\|\| \|\|\|\|\|\|\|\|\|\|\|\|\|+\|\|\|\|+\|\|\|\|\| \|\|+\|\| \| \|\|\|\|\|\|\|\|\|\|\|\|\| | |
| Sbjct: | 456 | SLEESVVDLDGRLPFVRPLPHIAVLRDELPRLFQDD-YGAEEEAAAAELRGEHTLTEKFV | 514 |
| Query: | 60 | CLDDSFGHDCSLTCDDCRNGGTCLLGLDGCDCPEGWTGVICNEICPP | 106 |
| | | \|\|\| \|\|\|\|\|\|\|\|\|\|\|\|\|\|\|\|\|\|\| \| \|\|\|\|\|\|\|\|\|\|\|+\|\|\|\| \|\|\| | |
| Sbjct: | 515 | CLDHSFGHDCSLTCDDCRNGGTCFPGQDGCDCPEGWTGIICNETCPP | 561 |

The presence of identifiable domains in AMF6, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMFX RNA was derived using tissue sources including, but not limited to, proprietary database sources, public EST sources, literature sources, and/or RACE sources, as described in the Examples. AMF6 is expressed in several regions of rat brain.

The nucleic acids and proteins of AMF6 are useful in potential therapeutic applications implicated in various AMF-related pathologies and/or disorders. For example, a cDNA encoding the MEGF-like protein may be useful in gene therapy, and the MEGF-like protein may be useful when administered to a subject in need thereof. The novel nucleic acid encoding AMF6 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from: gastric and renal cell carcinoma, breast and ovarian cancer, and other diseases, disorders and conditions of the like. By way of nonlimiting example, the compositions of the present invention will have efficacy for treatment of patients suffering from gastric and renal cell carcinoma, breast and ovarian cancer. Additional AMF-related diseases and disorders are mentioned throughout the Specification.

Further, the protein similarity information, expression pattern, and map location for AMF6 suggests that AMF6 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF6 substances for use in therapeutic or diagnostic methods. These antibodies may be generated according to methods known in the art, using prediction from hydrophobicity charts, as described in the “Anti-AMFX Antibodies” section below. In various embodiments, contemplated AMF6 epitopes are hydrophilic regions of the AMF6 polypeptide as predicted by software programs well known in the art that generate hydrophobicity or hydrophilicity plots.

AMF-7 (Also Referred to as Acc. No. 4194093)

Novel AMF7 is an Interleukin-11-like (“IL-11”) protein. The AMF7 clone is alternatively referred to herein as Acc. No. 4194093. The AMF7 nucleic acid (SEQ ID NO:13) of 1332 nucleotides is shown in Table 7A. The AMF7 open reading frame (“ORF”) begins at nucleotides 2-4. The AMF7 ORF terminates at a TGA codon at nucleotides 1307-1309. AMF7 appears to be a C-terminal fragment, so it is contemplated that the ORF extends beyond the N-terminus. As shown in Table 7A, putative untranslated regions 5′ to the start codon and 3′ to the stop codon are underlined, and the first coding triplet and the stop codon are in bold letters.

TABLE 7A
AMF7 nucleotide sequence (SEQ ID NO:13).

CGCCTTCATGCTGCCGGCGGGCTGCTCGCGCCGGCTGGTGGCCGAGCTGCAGGGCGCCCTGGACGCCTGCGCACAG
CGACAATTGCAATTGGAGCAGAGCCTGCGCGTTTGCCGTCGGCTGCTGCATGCCTGGGAACCAACTGGGACCCGGG
CTTTGAAGCCACCTCCAGGGCCAGAAACTAATGGAGAGGACCCCCTTCCAGCATGCACACCCAGTCCACAAGACCT
CAAAGAGTTGGAGTTTCTGACCCAGGCACTGGAGAAGGCTGTACGAGTTCGAAGAGGCATCACTAAGGCCGAAGAG
AGAGACAAGGCCCCCAGCCTGAAATCTAGGTCCATTGTCACCTCTTCTGGCACGACAGCCTCCGCCCCACCGCATT
CCCCAGGCCAAGCTGGTGGCCATGCTTCAGACACGAGACCCACCAAGGGCCTCCGCCAGACCACGGTGCCTGCCAA
GGGCCACCCTGAGCGCCGGCTGCTGTCAGTGGGGGATGGGACCCGTGTTGGGATGGGAGCCCGAACCCCCAGGCCT
GGGGCGGGCCTCAGGGACCAGCAAATGGCCCCATCCGCTGCTCCTCAGGCCCCAGAAGCCTTCACACTCAAGGAGA
AGGGGCACCTGCTGCGGCTGCCTGCGGCATTCAGGAAAGCAGCTTCCCAGAACTCGAGCCTGTGGGCCCAGCTCAG
TTCCACACAGACCAGTGATTCCACGGATGCCGCCGCTGCCAAAACCCAGTTCCTCCAGAACATGCAGACAGCTTCA
GGCGGGCCCCAGCCCAGGCTCAGTGCTGTGGAGGTGGAGGCGGAGGCGGGGCGCCTGCGGAAGGCCTGCTCGCTGC
TGAGACTGCGCATGAGGGAGGAGCTCTCAGCAGCCCCCATGGACTGGATGCAGGAgTACCGCTGCCTGCTCACGCT
GGAGGGGCTGCAGGCCATGGTGGGCCAGTGTCTGCACAGGCTGCAGGAGCTGCGTGCAGCGGTGGCGGAACAGCCA
CCAAGACCATGTCCTGTGGGGAGGCCCCCCGGAGCCTCGCCGTCCTGTGGGGGTAGAGCGGAGCCTGCATGGAGCC
CCCAGCTGCTTGTCTACTCCAGCACCCAGGAGCTGCAGACCCTGGCGGCCCTCAAGCTGCGAGTGGCTGTGCTGGA
CCAGCAGATCCACTTGGAAAAGGTCCTGATGGCTGAACTCCTCCCCCTGGTAAGCGCTGCACAGCCGCAGGGGCCG
CCCTGGCTGGCCCTGTGCCGGGCTGTGCACAGCCTGCTCTGCGAGGGAGGAGCACGTGTCCTTACCATCCTGCGGG
ATGAACCTGCAGTCTGAGCCTTTCCCATGCTGCCCTCGGC

The encoded AMF7 protein (SEQ ID NO:14) is a 435 amino acid protein shown in Table 7B.

TABLE 7B
AMF7 amino acid sequence (SEQ ID NO:14)

AFMLPAGCSRRLVAELQGALDACAQRQLQLEQSLRVCRRLLHAWEPTGTRALKPPPGPETNGEDPLPACTPSPQDL
KELEFLTQALEKAVRVRRGITKAEERDKAPSLKSRSIVTSSGTTASAPPHSPGQAGGHASDTRPTKGLRQTTVPAK
GHPERRLLSVGDGTRVGMGARTPRPGAGLRDQQMAPSAAPQAPEAFTLKEKGHLLRLPAAFRKAASQNSSLWAQLS
STQTSDSTDAAAAKTQFLQNMQTASGGPQPRLSAVEVEAEAGRLRKACSLLRLRMREELSAAPMDWMQEYRCLLTL
EGLQAMVGQCLHRLQELRAAVAEQPPRPCPVGRPPGASPSCGGRAEPAWSPQLLVYSSTQELQTLAALKLRVAVLD
QQIHLEKVLMAELLPLVSAAQPQCPPWLALCRAVHSLLCEGGARVLTILRDEPAV

In an analysis of public nucleic acid sequence databases, it was found, for example, that a fragment of the AMF7 nucleic acid sequence has 1299 of 1300 bases (99%) identical to a Homo sapiens cDNA FLJ13909 fis, clone Y79AA1000065 (GenBank Acc. No. AK023971) (SEQ ID NO:78) shown in Table 7C.

TABLE 7C
BLASTN of AMF7 against cDNA FLJ13909 (SEQ ID NO:78)

>AK023971 AK023971 Homo sapiens cDNA FLJ13909 fis, clone Y79AA1000065.
9/2000 Length = 1708 Strand = Plus/Plus
Score = 2569 bits (1296), Expect = 0.0
Identities = 1299/1300 (99%)

| | | | |
|---|---|---|---|
| Query: | 33 | ggctggtggccgagctgcagggcgccctggacgcctgcgcacagcgacaattgcaattgg | 92 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 138 | ggctggtggccgagctgcagggcgccctggacgcctgcgcacagcgacaattgcaattgg | 197 |
| Query: | 93 | agcagagcctgcgcgtttgccgtcggctgctgcatgcctgggaaccaactgggacccggg | 152 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 198 | agcagagcctgcgcgtttgccgtcggctgctgcatgcctgggaaccaactgggacccggg | 257 |
| Query: | 153 | ctttgaagccacctccagggccagaaactaatggagaggacccccttccagcatgcacac | 212 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 258 | ctttgaagccacctccagggccagaaactaatggagaggacccccttccagcatgcacac | 317 |
| Query: | 213 | ccagtccacaagacctcaaagagttggagtttctgacccaggcactggagaaggctgtac | 272 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 318 | ccagtccacaagacctcaaagagttggagtttctgacccaggcactggagaaggctgtac | 377 |
| Query: | 273 | gagttcgaagaggcatcactaaggccgaagagagagacaaggcccccagcctgaaatcta | 332 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\| | |
| Sbjct: | 378 | gagttcgaagaggcatcactaaggccggagagagagacaaggcccccagcctgaaatcta | 437 |
| Query: | 333 | ggtccattgtcacctcttctggcacgacagcctccgccccaccgcattccccaggccaag | 392 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 438 | ggtccattgtcacctcttctggcacgacagcctccgccccaccgcattccccaggccaag | 497 |
| Query: | 393 | ctggtggccatgcttcagacacgagacccaccaagggcctccgccagaccacggtgcctg | 452 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 498 | ctggtggccatgcttcagacacgagacccaccaagggcctccgccagaccacggtgcctg | 557 |
| Query: | 453 | ccaagggccaccctgagcgccggctgctgtcagtgggggatgggacccgtgttgggatgg | 512 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 558 | ccaagggccaccctgagcgccggctgctgtcagtgggggatgggacccgtgttgggatgg | 617 |
| Query: | 513 | gagcccgaacccccaggcctggggcgggcctcagggaccagcaaatggccccatccgctg | 572 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 618 | gagcccgaacccccaggcctggggcgggcctcagggaccagcaaatggccccatccgctg | 677 |
| Query: | 573 | ctcctcaggccccagaagccttcacactcaaggagaaggggcacctgctgcggctgcctg | 632 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 678 | ctcctcaggccccagaagccttcacactcaaggagaaggggcacctgctgcggctgcctg | 737 |
| Query: | 633 | cggcattcaggaaagcagcttcccagaactcgagcctgtgggcccagctcagttccacac | 692 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 738 | cggcattcaggaaagcagcttcccagaactcgagcctgtgggcccagctcagttccacac | 797 |
| Query: | 693 | agaccagtgattccacggatgccgccgctgccaaaacccagttcctccagaacatgcaga | 752 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 798 | agaccagtgattccacggatgccgccgctgccaaaacccagttcctccagaacatgcaga | 857 |
| Query: | 753 | cagcttcaggcgggccccagcccaggctcagtgctgtggaggtggaggcggaggcggggc | 812 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 858 | cagcttcaggcgggccccagcccaggctcagtgctgtggaggtggaggcggaggcggggc | 917 |
| Query: | 813 | gcctgcggaaggcctgctcgctgctgagactgcgcatgagggaggagctctcagcagccc | 872 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 918 | gcctgcggaaggcctgctcgctgctgagactgcgcatgagggaggagctctcagcagccc | 977 |
| Query: | 873 | ccatggactggatgcaggagtaccgctgcctgctcacgctggaggggctgcaggccatgg | 932 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 978 | ccatggactggatgcaggagtaccgctgcctgctcacgctggaggggctgcaggccatgg | 1037 |
| Query: | 933 | tgggccagtgtctgcacaggctgcaggagctgcgtgcagcggtggcggaacagccaccaa | 992 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1038 | tgggccagtgtctgcacaggctgcaggagctgcgtgcagcggtggcggaacagccaccaa | 1097 |
| Query: | 993 | gaccatgtcctgtggggaggccccccggagcctcgccgtcctgtgggggtagagcggagc | 1052 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1098 | gaccatgtcctgtggggaggccccccggagcctcgccgtcctgtgggggtagagcggagc | 1157 |
| Query: | 1053 | ctgcatggagcccccagctgcttgtctactccagcacccaggagctgcagaccctggcgg | 1112 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1158 | ctgcatggagcccccagctgcttgtctactccagcacccaggagctgcagaccctggcgg | 1217 |
| Query: | 1113 | ccctcaagctgcgagtggctgtgctggaccagcagatccacttggaaaaggtcctgatgg | 1172 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1218 | ccctcaagctgcgagtggctgtgctggaccagcagatccacttggaaaaggtcctgatgg | 1277 |
| Query: | 1173 | ctgaactcctccccctggtaagcgctgcacagccgcaggggccgccctggctggccctgt | 1232 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1278 | ctgaactcctccccctggtaagcgctgcacagccgcaggggccgccctggctggccctgt | 1337 |
| Query: | 1233 | gccgggctgtgcacagcctgctctgcgagggaggagcacgtgtccttaccatcctgcggg | 1292 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1338 | gccgggctgtgcacagcctgctctgcgagggaggagcacgtgtccttaccatcctgcggg | 1397 |
| Query: | 1293 | atgaacctgcagtctgagcctttcccatgctgccctcggc | 1332 |
| | | \|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1398 | atgaacctgcagtctgagcctttcccatgctgccctcggc | 1437 |

A BLASTP search was performed against public protein databases. As shown in Table 7D, the AMF7 protein has 78 of 332 amino acid residues (23%) identical to, and 113 of 332 residues (34%) positive with, the 1151 amino acid residue long Gallus gallus (chicken). high molecular mass nuclear antigen (fragment) (Acc. No. 057580) (SEQ ID NO:79).

TABLE 7D
BLASTP of AMF7 against chicken HMMNA (SEQ ID NO:79)

O57580 gallus gallus (chicken). high molecular mass nuclear antigen (fragment).
11/1998 Length = 1151
Score = 43.8, bits (101.0), Expect = 0.002
Identities = 78/332 (23%), Positives = 113/332, (34%)

| | | | |
|---|---|---|---|
| Query: | 44 | WEPTGTRALKPPPGPE-TNGEDPLPACTPSPQDLKELEFLTQALEKAVRVRRGITKAEER | 102 |
| | | \| \| \| \|\|\|\| \| \| \| +\|+ \|\| \| + + \| \| | |
| Sbjct: | 52 | WVPIG--GAPPPPGTEPTPPSKPTDGADAAPKASAELTSPPPASPSPPDGPKAPSGAGEA | 109 |
| Query: | 103 | DKAPSLKSRSIVTSSGTTASAPPHSPGQAGGSVGDGTRVGMGART---PRPGAGLRDQQM | 159 |
| | | + \|+ \| \|\| \| \| \|\| \|\|\|\| \| ++ +\| \| + | |
| Sbjct: | 110 | EAGTPPPSQG-------PAGTPPPSQGAAGAPKGDGTAQPSGTKSGADGKPAAQDVPKAT | 162 |
| Query: | 160 | AHASDTRPTKGLRQTTVPAKGHPERRLLPSAAPQAPEAFTLKEKGHLLRLPAAFRKAASQ | 219 |
| | | \|++ \|\| \|\|\| \| \| + +\|+ \|\|+\| \| \|\| \|\|\|\| | |
| Sbjct: | 163 | TAATEARPASA-ASPTVP-KATAEATAVTAASQSAPKAAT----------DAAAVTAASQ | 210 |
| Query: | 220 | NS-SLWAQLSSTQTSDSTDAAAAKTQFLQNMQTASGGPQPRLS----------------- | 261 |
| | | ++ ++ + + +\| \| \|\| \| \| \| | |
| Sbjct: | 211 | SAPKATVEVKPAAAAVAKEAKAVTAAAAAPKATAEAKPAPVTSPTIPCSSAEAKPLTAAS | 270 |
| Query: | 262 | --AVEVEAEAGRLRKACSLLRLRMREELSAAPMDWMQEYRCLLTLEGLQAMVGQCLHRLQ | 319 |
| | | \| + \|\|\| + \|\|+ ++ \| \|\| + + + + \| + ++ | |
| Sbjct: | 271 | PTASKATAEAKPVPATASLMATKVTAEAKPAPSPSVP--KATTDTKAVTATAPKAGPDVK | 328 |
| Query: | 320 | ELRAAVAEQPPRPCPVGRPPGASPSCGGRAEP | 351 |
| | | \| \|\| \| \| \| \|\| \| \| \| | |
| Sbjct: | 329 | PAVAVCAEAKPAPPP---PPQQLPKAAAAAAP | 357 |

AMF7 also is 16% identical to and 21% positive with Interleukin-11 Precursor (IL-11) (Adipogenesis InhibitorY Factor) (AGIF) (GenBank Acc. No. P20809) (SEQ ID NO:80) shown in Table 7E.

TABLE 7E
BLASTP of AMF7 against IL-11 Precursor (SEQ ID NO:80)

Identities: 0.16; Similarities: 0.21; Similarity Matrix: BLOSUM62

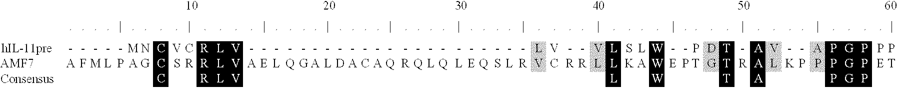

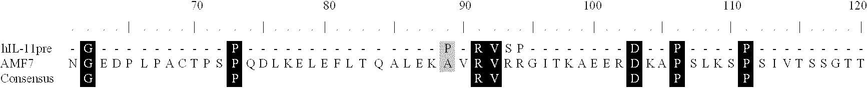

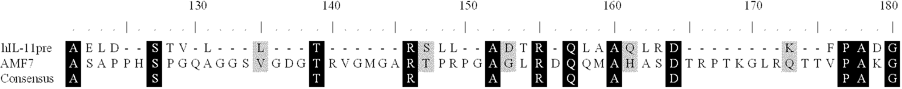

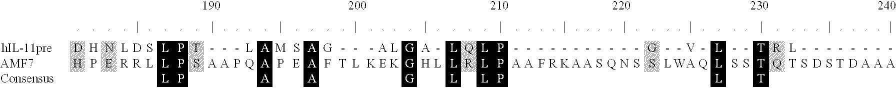

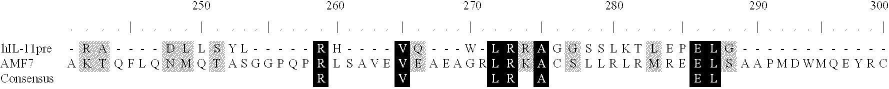

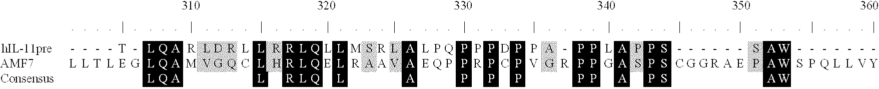

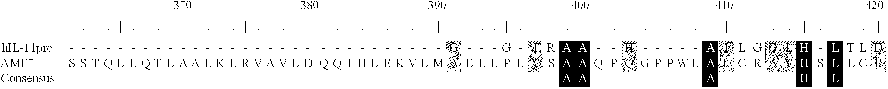

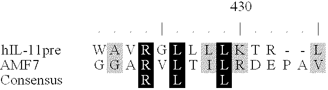

The presence of identifiable domains in AMF7, as well as all other AMFX proteins, can be determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results can then be collected from the Conserved Domain Database (CDD) with Reverse Position Specific BLAST analyses. This BLAST analysis software samples domains found in the Smart and Pfam collections.

Expression information for AMFX RNA was derived using tissue sources including, but not limited to, proprietary database sources, public EST sources, literature sources, and/or RACE sources, as described in the Examples. AMF7 is expressed in at least the following tissues: colon, ovarian, lung, renal and breast cancer. The expression in lung and renal cancer cell lines correlates with expression in the fetal tissues, indicating a oncofetal phenotype.

The nucleic acids and proteins of AMF7 are useful in potential therapeutic applications implicated in various AMF-related pathologies and/or disorders. For example, a cDNA encoding the IL-11-like protein may be useful in gene therapy, and the IL-11-like protein may be useful when administered to a subject in need thereof. The novel nucleic acid encoding AMF7 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from: diseases involving the growth of hematopoietic progenitor cells and platelet maturation, lung and renal cancer, and other diseases, disorders and conditions of the like. By way of nonlimiting example, the compositions of the present invention will have efficacy for treatment of patients suffering from diseases involving the growth of hematopoietic progenitor cells and platelet maturation, lung and renal cancer. Additional AMF-related diseases and disorders are mentioned throughout the Specification.

Further, the protein similarity information, expression pattern, and map location for AMF7 suggests that AMF7 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF7 substances for use in therapeutic or diagnostic methods. These antibodies may be generated according to methods known in the art, using prediction from hydrophobicity charts, as described in the “Anti-AMFX Antibodies” section below. In various embodiments, contemplated AMF7 epitopes are hydrophilic regions of the AMF7 polypeptide as predicted by software programs well known in the art that generate hydrophobicity or hydrophilicity plots.

AMF-8 (Also Referred to as Acc. No. AC01136_A)

AMF1 is a novel pleitrophin-like polypeptide. The AMF1 clone is alternatively referred to herein Acc. No. AC01136_A. The AMF1 nucleic acid (SEQ ID NO:15) of 510 nucleotides is shown in Table 8A. The AMF1 open reading frame (“ORF”) (SEQ ID NO: 16) begins at nucleotide 1. The AMF1 ORF terminates at a TAA codon at nucleotides 510-513. The AMF1 protein was predict to be a secreted protein.

TABLE 8A
AMF-8 DNA (SEQ ID NO:15) AND POLYPEPTIDE (SEQ ID NO:16)

| | |
|---|---|
| Translated Protein-Frame: 1-Nucleotide 1 to 510 | |
| ATGCAGGCTCAACAGTACCAGCAGCAGCGTCGAAAATTTGCAGCTGCCTTCTTGGCATTCATTTTCATACTGGCAGCTGT | 80 |
| M Q A Q Q Y Q Q Q R R K F A A A F L A F I F I L A A V | |
| GGATACTGCTGAAGCAGGGAAGAAAGAGAAACCAGAAAAAAAAGTGAAGAAGTCTGACTGTGGAGAATGGCAGTGGAGTG | 160 |
| D T A E A G K K E K P E K K V K K S D C G E W Q W S V | |
| TGTGTGTGCCCACCAGTGGAGACTGTGGGCTGGGCACACGGGAGGGCACTCGGACTGGAGCTGAGTGCAAGCAAACCATG | 240 |
| C V P T S G D C G L G T R E G T R G A E C K Q T M | |
| AAGACCCAGAGATGTAAGATCCCCTGCAACTGGAAGAAGCAATTTGGCGCGGAGTGCAAATACCAGTTCCAGGCCTGGGG | 320 |
| K T Q R C K I P C N W K K Q F G A E C K Y Q F Q A W G | |
| AGAATGTGACCTGAACACAGCCCTGAAGACCAGAACTGGAAGTCTGAAGCGAGCCCTGCACAATGCCGAATGCCAGAAGA | 400 |
| E C D L N T A L K T R T G S L K R A L H N A E C Q K T | |
| CTGTCACCATCTCCAAGCCCTGTGGCAAACTGACCAAGCCCAAACCTCAAGGTACCCTAGAACTTAAAGTAAAAAAAAAA | 480 |
| V T I S K P C G K L T K P K P Q G T L E L K V K K K | |
| AAAAAAAAAAAAAATTCTGAGGAGACCTTTTAG | 513 |
| K K K K N S E E T F | |

BLASTN information for AMF8-related nucleic acid sequences is shown in Table 8B.

TABLE 8B
BLASTN analysis results for AMF8

| | Score | E |
|---|---|---|
| Sequences producing significant alignments: | (bits) | Value |
| HUMHBNF1 M57399 Human nerve growth factor (HBNF-1) mRNA, com . . . | 894 | 0.0 |
| HSHBGF8 X52946 Human pleiotrophin (PTN) mRNA. 9/1993 | 894 | 0.0 |
| AB004306 AB004306 Homo sapiens mRNA for osteoblast stimulati . . . | 894 | 0.0 |
| D89546 D89546 Porcine mRNA for pleiotrophic factor beta, com . . . | 618 | e−175 |
| BTHBGF8 X52945 Bovine pleiotrophin (PTN) mRNA. 9/1993 | 609 | e−172 |
| RATHBGAM M55601 R. norvegicus heparin-binding growth associat . . . | 531 | e−148 |
| MUSOSF1 D90225 Mouse mRNA for OSF-1. 6/1999 | 502 | e−139 |

In an analysis of public nucleic acid sequence databases, it was found, for example, that the AMF1 nucleic acid sequence has 541/541 bases (100%) identical to human nerve growth factor (GenBank Acc. No. M57399) (SEQ ID NO:81) shown in Table 8C. In all BLAST alignments herein, the “E-value” or “Expect” value is a numeric indication of the probability that the aligned sequences could have achieved their similarity to the BLAST query sequence by chance alone, within the database that was searched. For example, as shown in Table 8B, the probability that the subject (“Sbjct”) retrieved from the AMF1 BLAST analysis, in this case the human nerve growth factor gene, matched the Query AMF1 sequence purely by chance is zero as shown by the E value of 0.0.

TABLE 8C
BLASTN of AMF1 against human NGF (SEQ ID NO:81)

>HUMHBNF1 M57399 Human nerve growth factor (HBNF-1) mRNA, complete cds. 4/1993
Length = 1029; Strand Plus/Plus
Score = 894 bits (451), Expect = 0.0
Identities = 451/451 (100%)

| | | | |
|---|---|---|---|
| Query: | 1 | atgcaggctcaacagtaccagcagcagcgtcgaaaatttgcagctgccttcttggcattc | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 396 | atgcaggctcaacagtaccagcagcagcgtcgaaaatttgcagctgccttcttggcattc | 455 |
| Query: | 61 | attttcatactggcagctgtggatactgctgaagcagggaagaaagagaaaccagaaaaa | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 456 | attttcatactggcagctgtggatactgctgaagcagggaagaaagagaaaccagaaaaa | 515 |
| Query: | 121 | aaagtgaagaagtctgactgtggagaatggcagtggagtgtgtgtgtgcccaccagtgga | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 516 | aaagtgaagaagtctgactgtggagaatggcagtggagtgtgtgtgtgcccaccagtgga | 575 |
| Query: | 181 | gactgtgggctgggcacacgggagggcactcggactggagctgagtgcaagcaaaccatg | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 576 | gactgtgggctgggcacacgggagggcactcggactggagctgagtgcaagcaaaccatg | 635 |
| Query: | 241 | aagacccagagatgtaagatcccctgcaactggaagaagcaatttggcgcggagtgcaaa | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 636 | aagacccagagatgtaagatcccctgcaactggaagaagcaatttggcgcggagtgcaaa | 695 |
| Query: | 301 | taccagttccaggcctggggagaatgtgacctgaacacagccctgaagaccagaactgga | 360 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 696 | taccagttccaggcctggggagaatgtgacctgaacacagccctgaagaccagaactgga | 755 |
| Query: | 361 | agtctgaagcgagccctgcacaatgccgaatgccagaagactgtcaccatctccaagccc | 420 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 756 | agtctgaagcgagccctgcacaatgccgaatgccagaagactgtcaccatctccaagccc | 815 |
| Query: | 421 | tgtggcaaactgaccaagcccaaacctcaag | 451 |
| | | \|\|\|\|\|\|\|\|\|\|\|\|\|\|\|\|\|\|\|\|\|\|\|\|\|\|\|\|\|\|\| | |
| Sbjct: | 816 | tgtggcaaactgaccaagcccaaacctcaag | 846 |

A BLASTP search was performed against public protein databases. The results from this comparison are shown in Table 8D.

TABLE 8D
BLASTP analysis results for AMF8

| | Score | E |
|---|---|---|
| Sequences producing significant alignments: | (bits) | Value |
| FGFJ_HUMAN O95750 homo sapiens (human) . fibroblast growth fa . . . | 92 | 2e−18 |
| O95750 O95750 homo sapiens (human) . fgf-19. 5/1999 | 92 | 2e−18 |
| FGFF_MOUSE O35622 mus musculus (mouse) . fibroblast growth fa . . . | 79 | 1e−14 |
| FGF3_MOUSE P05524 mus musculus (mouse) . int-2 proto-oncogene . . . | 71 | 5e−12 |
| FGF3_HUMAN P11487 homo sapiens (human) . int-2 proto-oncogene . . . | 70 | 8e−12 |

For example, as shown in Table 8E, the AMF8 protein has 57 of 143 amino acid residues (39%) identical to, and 79 of 143 residues (54%) positive with, the 216 amino acid residue long human fibroblast growth factor. (Acc. No. 095750) (SEQ ID NO:82).

### Table 8E. BLASTP of AMF1 against human FGF (SEQ ID NO:82)

>FGFJ_HUMAN 095750 homo sapiens (human). fibroblast growth factor-19 precursor (fgf-19). 10/2000 Length=216

Score=92.1 bits (225), Expect=2e-18

Identities=57/143 (39%), Positives=79/143 (54%), Gaps=6/143 (4%)

TABLE 8E
BLASTP of AMF1 against human FGF (SEQ ID NO:82)

>FGFJ_HUMAN 095750 homo sapiens (human). fibroblast growth factor-19
precursor (fgf-19). 10/2000 Length = 216
Score = 92.1 bits (225), Expect = 2e − 18
Identities = 57/143 (39%), Positives = 79/143 (54%), Gaps = 6/143 (4%)

| | | | |
|---|---|---|---|
| Query: | 15 | VSVLAGLLLGACQAHPIP--DSSPLLQFG--GQVRQRYLYTDDAQQ-TEAHLEIREDGTV | 69 |
| | | \| +\|\|\|\| \| \| \|+ \|+ \| + +\| +\| \|+\|\|\| + \| \|\| \|\| \| | |
| Sbjct: | 10 | VWILAGLWL-AVAGRPLAFSDAGPHVHYGWGDPIRLRHLYTSGPHGLSSCFLRIRADGVV | 68 |
| Query: | 70 | GGAADQSPESLLQLKALKPGVIQILGVKTSRFLCQRPDGALYGSLHFDPEACSFRELLLE | 129 |
| | | \| \|\| \|\|\|++\|\|+ + \| \|\| + \|+\|\| \|\| + \| \| + \| \|+\| \| + | |
| Sbjct: | 69 | DCARGQSAHSLLEIKAVALRTVAIKGVHSVRYLCMGADGKMQGLLQYSEEDCAFEEEIRP | 128 |
| Query: | 130 | DGYNVYQSEAHGLPLHLPGLQRR | 152 |
| | | \|\|\|\|\|\|+\|\| \| \|\|+ \| ++\| | |
| Sbjct: | 129 | DGYNVYRSEKHRLPVSLSSAKQR | 151 |

Expression information for AMFX RNA was derived using tissue sources including, but not limited to, proprietary database sources, public EST sources, literature sources, and/or RACE sources, as described in the Examples. AMF1 is expressed in at least the following tissues, several brain tumor cell lines and fetal derived tissue. The nucleic acids and proteins of AMF1 are useful in potential therapeutic applications implicated in various AMF-related pathologies and/or disorders. For example, a cDNA encoding the pleiotrophin-like protein may be useful in gene therapy, and the pleiotrophin-like protein may be useful when administered to a subject in need thereof. The novel nucleic acid encoding AMF1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from cancer and other cell proliferative disorders. By way of nonlimiting example, the compositions of the present invention will have efficacy for treatment of patients suffering from cancer and other cell proliferative disorders. Additional AMF-related diseases and disorders are mentioned throughout the Specification.

Further, the protein similarity information, expression pattern, and map location for AMF1 suggests that AMF1 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF1 substances for use in therapeutic or diagnostic methods. These antibodies may be generated according to methods known in the art, using prediction from hydrophobicity charts, as described in the “Anti-AMFX Antibodies” section below. In various embodiments, contemplated AMF1 epitopes are hydrophilic regions of the AMF1 polypeptide as predicted by software programs well known in the art that generate hydrophobicity or hydrophilicity plots.

AMF-9 (Also Referred to as Acc. No. AL307658)

AMF9 is a novel GPCR-like polypeptide. The AMF9 clone is alternatively referred to herein Acc. No. AL307658. The AMF9 nucleic acid (SEQ ID NO:17) is shown in Table 9A.

The AMF9 open reading frame (“ORF”) (SEQ ID NO: 18) encodes for a 94 amino acid protein. The AMF9 polypeptide is encoded in a negative reading frame. The sequence shown below has been reverse-complemented and renumbered to allow reading of the protein in the expected N to C direction.

TABLE 9A
AMF-9 DNA (SEQ ID NO:17) and Polypeptide (SEQ ID NO:18)

| Translated Protein - Frame: -1 - Nucleotide 16 to 297 | |
|---|---|
| CGAAGGGCTTTCACAATGCTAGGTGTGGTCTGGCTGGTGGCAGTCATCGTAGGATCACCCATGTGGCACGTGCAACAACT | 80 |
| M L G V V W L V A V I V G S P M W H V Q Q L | |
| TGAGATCAAATATGACTTCCTATATGAAAAGGAACACATCTGCTGCTTAGAAGAGTGGACCAGCCCTGTGCACCAGAAGA | 160 |
| E I K Y D F L Y E K E H I C C L E E W T S P V H Q K I | |
| TCTACACCACCTTCATCCTTGTCATCCTCTTCCTCCTGCCTCTTA\overline{\|TGGAAGAAGAAACGAGCTGTCA\|}TTATGATGGTGAC | 240 |
| Y T T F I L V I L F L L P L M E E E T S C H Y D G D | |
| AGTGGTGGCTCTCTTTGCTGTG\overline{\|TGCTGGGCACCATTCCAT\|}GTTGTCCATATGATGATTGAATACAGTAATTTTGAAAAGG | 320 |
| S G G S L C C V L G T I P C C P Y D D | |
| AATATGATGATGTCACAATCAAGATGATTTTTGCTATCGTGCAAATTATTGGATTTTCCAACTCCATCTGTAATCCCATT | 400 |
| GTCTATGCATTTATGAATGAAAACTTCAAAAA | 432 |

A BLASTN analysis produced no significant homologies, as shown in Table 9B below. In all BLAST alignments herein, the “E-value” or “Expect” value is a numeric indication of the probability that the aligned sequences could have achieved their similarity to the BLAST query sequence by chance alone, within the database that was searched.

TABLE 9B
BLASTN alignment results for AMF9

| Matching | | | | |
|---|---|---|---|---|
| Entry (in | | | | |
| GenBank | Begin- | | | E |
| Main) | End | Description | Score | Value |
| gb:AL079305 | [255- | Human chromosome 14 DNA sequence *** | 44.1 | 0.059 |
| CNS00M8M | 276] | IN PROGRESS *** | | |
| | | BAC R-306B9 of library RPCI-11 from | | |
| | | chromosome 14 of Homo sapiens (Human), | | |
| | | complete sequence. | | |
| gb:AP001729 | [219- | Homo sapiens genomic DNA, chromosome | 44.1 | 0.059 |
| AP001729 | 240] | 21q, section 73/105. | | |
| gb:AP001436 | [219- | Homo sapiens genomic DNA, chromosome | 44.1 | 0.059 |
| AP001436 | 240] | 21q22.2, clone:T556, | | |
| | | LB7T-ERG region, complete sequence. | | |
| gb:AP000156 | [219- | Homo sapiens genomic DNA, chromosome | 44.1 | 0.059 |
| AP000156 | 240] | 21q22.2, DSCR region, clone D47-S479, | | |
| | | segment 8/16, complete sequence. | | |
| gb:AP000014 | [219- | Homo sapiens genomic DNA of 21q22.2 | 44.1 | 0.059 |
| AP000014 | 240] | Down Syndrome region, segment 7/13. | | |
| gb:L21977 | [276- | Petunia hybrida potential | | |
| PETACO2A | 297] | 1-aminocyclopropane-1-carboxylate | 44.1 | 0.059 |
| | | oxidase (ACO2) pseudogene sequence. | | |

A BLASTP search was performed against public protein databases. The results from this comparison are shown in Table 9C. In both Table 9B and Table 9C, as indicated by the fact that all resulting E values are higher than 0.001, no database entries were identified that had highly significant homologies to AMF9, i.e., that at least one subject sequence within the public databases searched would have homology to the AMF9 Query sequence, due to chance alone, would be more frequent than 1 in 1000.

TABLE 9C
BLASTP alignment results for AMF9

| Matching Entry | | | | |
|---|---|---|---|---|
| (in SwissProt + | Begin- | | | E |
| SpTrEMBL) | End | Description | Score | Value |
| spt:Q62805 | [2-64] | GALANIN RECEPTOR TYPE 1 (GAL 1-R) (GALR1). | 40.2 | 0.003 |
| GALR_RAT | | | | |
| spt:P56479 | [2-64] | GALANIN RECEPTOR TYPE 1 (GAL 1-R) (GALR1). | 40.2 | 0.003 |
| GALR_MOUSE | | | | |
| spt:P50391 | [4-63] | NEUROPEPTIDE Y RECEPTOR TYPE 4 (NPY4-R) | 39.1 | 0.008 |
| NY4R_HUMAN | | (PANCREATIC POLYPEPTIDERECEPTOR 1) (PP1). | | |
| spt:Q9Z2D4 | [4-63] | PANCREATIC POLYPEPTIDE RECEPTOR Y4. | 39.1 | 0.008 |
| Q9Z2D4 | | | | |
| spt:Q61041 | [4-63] | NEUROPEPTIDE Y RECEPTOR TYPE 4 (NPY4-R) | 37.9 | 0.017 |
| NY4R_MOUSE | | (PANCREATIC POLYPEPTIDERECEPTOR 1) (PP1) | | |
| | | (NPYR-D). | | |
| spt:O73734 | [2-64] | NEUROPEPTIDE Y/PEPTIDE YY RECEPTOR YC. | 37.5 | 0.023 |
| O73734 | | | | |
| spt:O97505 | [4-63] | NEUROPEPTIDE Y RECEPTOR TYPE 4. | 37.5 | 0.023 |
| O97505 | | | | |
| spt:Q22995 | [3-62] | SIMILAR TO FAMILY 1 OF G-PROTEIN COUPLED | 37.5 | 0.023 |
| Q22995 | | RECEPTORS. | | |

For example, as shown in Table 9D, the AMF9 protein has 18 of 63 amino acid residues (29%) identical to, and 33 of 63 residues (52%) positive with, the 346 amino acid residue long rat galanin receptor type 1 (SEQ ID NO:83).

TABLE 9D
BLASTP of AMF9 against rat galanin receptor type 1 (SEQ ID NO:83)

GALR_RAT rattus norvegicus (rat). galanin receptor type 1 (gall-r)
(galr1). 7/1998
Length = 346, Score = 40.2, bits (92.0), Expect = 0.003
Identities = 18/63 (29%) , Positives = 33/63, (52%)

| | | | |
|---|---|---|---|
| Query: | 2 | LGVVWLVAVIVGSPMWHVQQLEIKYDFLYEKEHICCLEEWTSPVHQKIYTTFILVILFLL | 61 |
| | | +\| +\| +++ + \|\| + + \|+\| \| + \| \| \| + +\|+\| \| \| +\|\| | |
| Sbjct: | 155 | VGFIWALSIAMASPVAYYQRL-----FHRDSNQTFCWEHWPNQLHKKAYVVCTFVFGYLL | 209 |
| Query: | 62 | PLM | 64 |
| | | \|\|+ | |
| Sbjct: | 210 | PLL | 212 |

The nucleic acids and proteins of AMF9 are useful in potential therapeutic applications implicated in various AMF-related pathologies and/or disorders. For example, a cDNA encoding the GPCR-like protein may be useful in gene therapy, and the GPCR-like protein may be useful when administered to a subject in need thereof. The novel nucleic acid encoding AMF9 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from cancer and other cell proliferative disorders. By way of nonlimiting example, the compositions of the present invention will have efficacy for treatment of patients suffering from cancer and other cell proliferative disorders. Additional AMF-related diseases and disorders are mentioned throughout the Specification.

Further, the protein similarity information, expression pattern, and map location for AMF9 suggests that AMF9 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF9 substances for use in therapeutic or diagnostic methods. These antibodies may be generated according to methods known in the art, using prediction from hydrophobicity charts, as described in the “Anti-AMFX Antibodies” section below. In various embodiments, contemplated AMF9 epitopes are hydrophilic regions of the AMF9 polypeptide as predicted by software programs well known in the art that generate hydrophobicity or hydrophilicity plots.

AMF-10 (Also Referred to as Acc. No. G55707_A)

AMF10 is a novel growth/differentiation factor-6-like polypeptide. The AMF10 clone is alternatively referred to herein Acc. No. G55707_A The AMF10 nucleic acid (SEQ ID NO:19) of 1425 nucleotides is shown in Table 9A. The AMF10 open reading frame (“ORF”) (SEQ ID NO:20) begins at nucleotide 31. The AMF10 ORF terminates at a TAG codon at nucleotides 1396-1398. The AMF1O protein was predict to be a secreted protein. The program SignalP predicts a signal peptide with the most likely cleavage site between amino acids 22 and 23. The predicted molecular weight of the AMF10 polypeptide is 50677 Da.

TABLE 10A
AMF-10 DNA (SEQ ID NO:19) and Polypeptide (SEQ ID NO:20)

| | |
|---|---|
| CTC CTG GGG AGA CGC AGC CAC TTG CCC CCC ATG CAT ACT CCC AGG | 45 |
| Met Asp Thr Pro Arg | |
| GTC CTG CTC TCG GCC GTC TTC CTC ATC AGT TTT CTG TGG GAT TTG | 90 |
| Val Leu Leu Ser Ala Val Phe Leu Ile Ser Phe Leu Trp Asp Leu | |
| CCC GGT TTC CAG CAG GCT TCC ATC TCA TCC TCC TGT TCG TCC GCC | 135 |
| Pro Gly Phe Gln Gln Ala Ser Ile Ser Ser Ser Cys Ser Ser Ala | |
| GAG CTG GGT TCC ACC AAG GGC ATG CGA AGC CGC AAG GAA GGC AAG | 180 |
| Glu Leu Gly Ser Thr Lys Gly Met Arg Ser Arg Lys Glu Gly Lys | |
| ATG CAG CGG GCG CCG CGC GAC AGT GAC GCG GGC CGG GAG GGC CAG | 225 |
| Met Gln Arg Ala Pro Arg Asp Ser Asp Ala Gly Arg Glu Gly Gln | |
| GAA CCA CAG CCG CGG CCT CAG GAC GAA CCC CGG GCT CAG CAG CCC | 270 |
| Glu Pro Gln Pro Arg Pro Gln Asp Glu Pro Arg Ala Gln Gln Pro | |
| CGG GCG CAG GAG CCG CCA GGC AGG GGT CCG CGC GTG GTG CCC CAC | 315 |
| Arg Ala Gln Glu Pro Pro Gly Arg Gly Pro Arg Val Val Pro His | |
| GAG TAC ATG CTG TCA ATC TAC AGG ACT TAC TCC ATC GCT GAG AAG | 360 |
| Glu Tyr Met Leu Ser Ile Tyr Arg Thr Tyr Ser Ile Ala Glu Lys | |
| CTG GGC ATC AAT GCC AGC TTT TTC CAG TCT TCC AAG TCG GCT AAT | 405 |
| Leu Gly Ile Asn Ala Ser Phe Phe Gln Ser Ser Lys Ser Ala Asn | |
| ACG ATC ACC AGC TTT GTA GAC AGG GGA CTA GAC GAT CTC TCG CAC | 450 |
| Thr Ile Thr Ser Phe Val Asp Arg Gly Leu Asp Asp Leu Ser His | |
| ACT CCT CTC CGG AGA CAG AAG TAT TTG TTT GAT GTG TCC ATG CTC | 495 |
| Thr Pro Leu Arg Arg Gln Lys Tyr Leu Phe Asp Val Ser Met Leu | |
| TCA GAC AAA GAA GAG CTG GTG GGC GCG GAG CTG CGG CTC TTT CGC | 540 |
| Ser Asp Lys Glu Glu Leu Val Gly Ala Glu Leu Arg Leu Phe Arg | |
| CAG GCG CCC TCA GCG CCC TGG GGG CCA CCA GCC GGG CCG CTC CAC | 585 |
| Gln Ala Pro Ser Ala Pro Trp Gly Pro Pro Ala Gly Pro Leu His | |
| GTG CAG CTC TTC CCT TGC CTT TCG CCC CTA CTG CTG GAC GCG CGG | 630 |
| Val Gln Leu Phe Pro Cys Leu Ser Pro Leu Leu Leu Asp Ala Arg | |
| ACC CTG GAC CCG CAG GGG GCG CCG CCG GCC GGC TGG GAA GTC TTC | 675 |
| Thr Leu Asp Pro Gln Gly Ala Pro Pro Ala Gly Trp Glu Val Phe | |
| GAC GTG TGG CAG GGC CTG CGC CAC CAG CCC TGG AAG CAG CTG TGC | 720 |
| Asp Val Trp Gln Gly Leu Arg His Gln Pro Trp Lys Gln Leu Cys | |
| TTG GAG CTG CGG GCC GCA TGG GGC GAG CTG GAC GCC GGG GAG GCC | 765 |
| Leu Glu Leu Arg Ala Ala Trp Gly Glu Leu Asp Ala Gly Glu Ala | |
| GAG GCG CGC GCG CGG GGA CCC CAG CAA CCG CCG CCC CCG GAC CTG | 810 |
| Glu Ala Arg Ala Arg Gly Pro Gln Gln Pro Pro Pro Pro Asp Leu | |
| CGG AGT CTG GGC TTC GGC CGG AGG GTG CGG CCT CCC CAG GAG CGG | 855 |
| Arg Ser Leu Gly Phe Gly Arg Arg Val Arg Pro Pro Gln Glu Arg | |
| GCC CTG CTG GTG GTA TTC ACC AGA TCC CAG CGC AAG AAC CTG TTC | 900 |
| Ala Leu Leu Val Val Phe Thr Arg Ser Gln Arg Lys Asn Leu Phe | |
| GCA GAG ATG CGC GAG CAG CTG GGC TCG GCC GAG GCT GCG GGC CCG | 945 |
| Ala Glu Met Arg Glu Gln Leu Gly Ser Ala Glu Ala Ala Gly Pro | |
| GGC GCG GGC GCC GAG GGG TCG TGG CCG CCG CCG TCG GGC GCC CCG | 990 |
| Gly Ala Gly Ala Glu Gly Ser Trp Pro Pro Pro Ser Gly Ala Pro | |
| GAT GCC AGG CCT TGG CTG CCC TCG CCC GGC CGC CGG CGG CGG CGC | 1035 |
| Asp Ala Arg Pro Trp Leu Pro Ser Pro Gly Arg Arg Arg Arg Arg | |
| ACG GCC TTC GCC AGT CGC CAT GGC AAG CGG CAC GGC AAG AAG TCC | 1080 |
| Thr Ala Phe Ala Ser Arg His Gly Lys Arg His Gly Lys Lys Ser | |
| AGG CTA CGC TGC AGC AAG AAG CCC CTG CAC GTG AAC TTC AAG GAG | 1125 |
| Arg Leu Arg Cys Ser Lys Lys Pro Leu His Val Asn Phe Lys Glu | |
| CTG GGC TGG GAC GAC TGG ATT ATC GCG CCC CTG GAG TAC GAG GCC | 1170 |
| Leu Gly Trp Asp Asp Trp Ile Ile Ala Pro Leu Glu Tyr Glu Ala | |
| TAT CAC TGC GAG GGT GTA TGC GAC TTC CCG CTG CGC TCG CAC CTG | 1215 |
| Tyr Hls Cys Glu Gly Val Cys Asp Phe Pro Leu Arg Ser Hls Leu | |
| GAG CCC ACC AAC CAC GCC ATC ATC CAG ACG CTG ATG AAC TCC ATG | 1260 |
| Glu Pro Thr Asn His Ala Ile Ile Gln Thr Leu Met Asn Ser Met | |
| GAC CCC GGC TCC ACC CCG CCC AGC TGC TGC GTG CCC ACC AAA TTG | 1305 |
| Asp Pro Gly Ser Thr Pro Pro Ser Cys Cys Val Pro Thr Lys Leu | |
| ACT CCC ATC AGC ATT CTA TAC ATC GAC GCG GGC AAT AAT GTG GTC | 1350 |
| Thr Pro Ile Ser Ile Leu Tyr Ile Asp Ala Gly Asn Asn Val Val | |
| TAC AAG CAG TAC GAG GAC ATG GTG GTG GAG TCG TGC GGC TGC AGG | 1395 |
| Tyr Lys Gln Tyr Glu Asp Met Val Val Glu Ser Cys Gly Cys Arg | |
| TAG CGG TGC CTT TCC CGC CGC CTT GGC CCG | 1425 |

In an analysis of public nucleic acid sequence databases, it was found, for example, that the AMF10 nucleic acid sequence has 95/98 bases (96%) identical to bos taurus cartilage-derived morphogenic protein 2 (GenBank Acc. No. BTU13661) (SEQ ID NO:84) shown in Table 10B. In all BLAST alignments herein, the “E-value” or “Expect” value is a numeric indication of the probability that the aligned sequences could have achieved their similarity to the BLAST query sequence by chance alone, within the database that was searched.

TABLE 10B
BLASTN of AMF10 against CDMP 2 (SEQ ID NO:84)

>BTU13661 U13661 Bos taurus cartilage-derived morphogenetic protein 2
(CDMP-2) mRNA, complete cds. 1/1995,
Length = 1308; Strand Plus/Plus
Score = 170 bits (86), Expect = 8e − 41
Identities = 95/98 (96%)

| | | | |
|---|---|---|---|
| Query: | 3 | gacttactccatcgctgagaagctgggcatcaatgccagctttttccagtcttccaagtc | 62 |
| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | | |
| Sbjct: | 234 | gacttactccatcgccgagaagctgggcatcaatgctagctttttccagtcttccaagtc | 293 |
| Query: | 63 | ggctaatacgatcaccagctttgtagacaggggactag | 100 |
| \|\|\|\|\|\|\|\|\|\|\|\|\|\|\|\|\|\|\|\|\|\|\|\|\|\|\|\|\|\|\|\|\|\|\|\|\|\| | | | |
| Sbjct: | 294 | ggctaatacgatcactagctttgtagacaggggactag | 331 |

Additional BLASTN information for related nucleic acid sequences is shown in Table 10C.

TABLE 10C

| | Score | E |
|---|---|---|
| Sequences producing significant alignments: | (bits) | Value |
| BTU13661 U13661 Bos taurus cartilage-derived | 170 | 8e−41 |
| morphogenetic . . . | | |
| AC058786 AC058786 Mus musculus clone RP23-117o7, | 151 | 7e−35 |
| complete . . . | | |
| AF155125 AF155125 Xenopus laevis growth and | 56 | 3e−06 |
| differentiatio . . . | | |

A BLASTP search was performed against public protein databases. The result from this comparison are shown in Tables 10D. As shown in Table 10D, the AMF10 protein has 354 of 435 amino acid residues (81%) identical to, and 372 of 435 residues (85%) positive with, the 436 amino acid residue long bos taurus growth and differentiation factor 6 precursor. (Acc. No. P55106) (SEQ ID NO:85).

TABLE 10D
BLASTP of AMF10 against GDF 6 precursor (SEQ ID NO:85)

>ptnr:SWISSPROT-ACC:P55106 GROWTH/DIFFERENTIATION FACTOR 6 PRECURSOR
(GDF-6) (CARTILAGE-DERIVED MORPHOGENETIC PROTEIN 2) (CDMP-2) - Bos taurus
(Bovine), 436 aa (fragment).
Length = 436
Score = 1795 (631.9 bits), Expect = 6.3e − 185, P = 6.3e − 185
Identities = 354/435 (81%), Positives = 372/435 (85%)

| | | | |
|---|---|---|---|
| Query: | 33 | SSAELGSTKGMRSRKEGKMQRARAPRDSDAGREG---QEPQPRPQDEPRA---QQPRAQEPP | 86 |
| | | +\|\|\|\|\|\| \|\|\|\|+\|\|\|\|+\| \|\|\|\|++ \|\| \|\|\| \|\|\|\|+\|\|\|+ \|\|\| \|+\|\|\| | |
| Sbjct: | 2 | ASAELGSAKGMRTRKEGRMPRAPRENATAREPLDRQEPPPRPQEEPQRRPPQQPEAREPP | 61 |
| Query: | 87 | GRGPRVVPHEYMLSIYRTYSIAEKLGINASFFQSSKSANTITSFVDRGLDDLSHTPLRRQ | 146 |
| | | \|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 62 | GRGPRLVPHEYMLSIYRTYSIAEKLGINAFFQSSKSANTITSFVDRGLDDLSHTPLRRQ | 121 |
| Query: | 147 | KYLFDVSMLSDKEELVGAELRLFRQAPSAPWGPPAGPLHVQLFPCLSPLLLDARTLDPQG | 206 |
| | | \|\|\|\|\|\|\| \|\|\|\|\|\|\|\|\|\|++\|\|\|\|\|\|\|+\| \| \| \|\| \| ++\| \| \| \| | |
| Sbjct: | 122 | KYLFDVSTLSDKEELVGADVRLFRQAPAALAPPAAAPLAALRLP-VAPAAGSAEP-GPAG | 179 |
| Query: | 207 | APPAGWEVFDVWQGLRHQPWKQLCLELRAAWG-ELDAGEAEARPRARGPQQPPPPDLRSLGF | 265 |
| | | \|\| \|\|\|\|\|\|\|\|+\|\|\| \|\|\|\|\|\|\|\|\|\|\|\|\|\|\| \| \| \| \|\|\| \|\|\|\|\|\|\|\|\|\|\|\|\|\|\| | |
| Sbjct: | 180 | APRPGWEVFDVWRGLRPQPWKQLCLELRAAWGGEPGAAEDEARTPGPQQPPPPDLRSLGF | 239 |
| Query: | 266 | GRRVRPPQERALLVVFTRSQRKNLFAEMREQLGSA-EAAGPGAGAEGSWPPP-------S | 317 |
| | | \|\|\|\|\| \|\|\|\|\|\|\|\|\|\|+\|\|\|\|\| \|\|\|\|\|\|\|\|\|\|\|\| \| \|\|\| \|\|\|\|\| \|\|\|\| \| | |
| Sbjct: | 240 | GRRVRTPQERALLVVFSRSQRKTLFAEMREQLGSATEVVGPGGGAEGSGPPPPPPPPPPS | 299 |
| Query: | 318 | GAPDARPWLPSPGRRRRRTAFASRHGKRHGKKSRLRCSKKPLHVNFKELGGCWDDWIIAPLE | 377 |
| | | \| \|\|\| \| \|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 300 | GTPDAGLWSPSPGRRRR-TAFASRHGKRHGKKSRLRCSKKPLHVNFKELGWDDWIIAPLE | 358 |
| Query: | 378 | YEAYHCEGVCDFPLRSHLEPTNHAIIQTLMNSMDPGSTPPSCCVPTKLTPISILYIDAGN | 437 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 359 | YEAYHCEGVCDFPLRSHLEPTNHAIIQTLMNSMDPGSTPPSCCVPTKLTPISILYIDAGN | 418 |
| Query: | 438 | NVVYKQYEDMVVESCGCR | 455 |
| | | \|\|\|\| +\|\|+\|\|\|\|\|\|\|\|\| | |
| Sbjct: | 419 | NVVYNEYEEMVVESCGCR | 436 |

Expression information for AMFX RNA was derived using tissue sources including, but not limited to, proprietary database sources, public EST sources, literature sources, and/or RACE sources, as described in the Examples. AMF10 is expressed in at least, e.g., astrocytoma and glioma derived tissue. The nucleic acids and proteins of AMF10 are useful in potential therapeutic applications implicated in various AMF-related pathologies and/or disorders. For example, a cDNA encoding the growth/differentiation factor-6-like protein may be useful in gene therapy, and the growth/differentiation factor-6-like protein may be useful when administered to a subject in need thereof. The novel nucleic acid encoding AMF10 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The AMFX nucleic acids and proteins are useful in potential diagnostic and therapeutic applications implicated in various diseases and disorders described below and/or other pathologies. For example, the compositions of the present invention will have efficacy for treatment of patients suffering from cancer and other cell proliferative disorders. By way of nonlimiting example, the compositions of the present invention will have efficacy for treatment of patients suffering from cancer and other cell proliferative disorders. Additional AMF-related diseases and disorders are mentioned throughout the Specification.

Further, the protein similarity information, expression pattern, and map location for AMF10 suggests that AMF10 may have important structural and/or physiological functions characteristic of the AMF family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel AMF10 substances for use in therapeutic or diagnostic methods. These antibodies may be generated according to methods known in the art, using prediction from hydrophobicity charts, as described in the “Anti-AMFX Antibodies” section below. In various embodiments, contemplated AMF10 epitopes are hydrophilic regions of the AMF10 polypeptide as predicted by software programs well known in the art that generate hydrophobicity or hydrophilicity plots.

AMFX Nucleic Acids and Polypeptides

Novel AMFX nucleic acid and polypeptide sequences disclosed in the invention include those summarized in Table 11.

TABLE 11
AMFX Sequences and Corresponding SEQ ID Numbers

| AMFX | Internal | SEQ ID NO | SEQ ID NO | |
|---|---|---|---|---|
| No. | Identification | (nucleic acid) | (polypeptide) | Homology |
| 1 | 14209510 | 1 | 2 | Fibrillin 2 |
| | | | | precursor |
| 2 | 20421338 | 3 | 4 | Nephrin |
| 3 | 27251385 | 5 | 6 | Fibrillin 2 |
| | | | | precursor |
| 4 | 27486474 | 7 | 8 | Plasminogen |
| 5 | 29691387 | 9 | 10 | Organic Anion |
| | | | | Transporter |
| 6 | 12996895_1 | 11 | 12 | MEGF6 |
| 7 | 38905521 | 13 | 14 | IL-11 |
| 8 | AC11036_A | 15 | 16 | Pleiotrophin |
| 9 | AL307658 | 17 | 18 | GPCR13 |
| 10 | GMG55707_{—} | 19 | 20 | GDF6 |
| | EXT.0.1_da1 | | | |

One aspect of the invention pertains to isolated nucleic acid molecules that encode AMFX polypeptides or biologically-active portions thereof. Also included in the invention are nucleic acid fragments sufficient for use as hybridization probes to identify AMFX-encoding nucleic acids (e.g., AMFX mRNAs) and fragments for use as PCR primers for the amplification and/or mutation of AMFX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An AMFX nucleic acid can encode a mature AMFX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terninal methionine. Alternatively, a mature form arising from a precursor polypeptide or protein having residues 1 to N, in which an N-terminal signal sequence from residue I to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated AMFX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17 and 19, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17 and 19 as a hybridization probe, AMFX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to AMFX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 1, 3, 5, 7, 9, 11, 13, 15, 17 and 19, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17 and 19, or a portion of this nucleotide sequence (e.g., a fragment that can be used as a probe or primer or a fragment encoding a biologically-active portion of an AMFX polypeptide). A nucleic acid molecule that is complementary to the nucleotide sequence shown in SEQ ID NOS:1, 3, 5, 7, 9, 11, 13, 15, 17 and 19, is one that is sufficiently complementary to the nucleotide sequence shown in SEQ ID NOS:1, 3, 5, 7, 9, 11, 13, 15, 17 and 19, that it can hydrogen bond with little or no mismatches to the nucleotide sequence shown in SEQ ID NOS:1, 3, 5, 7, 9, 11, 13, 15, 17 and 19, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of AMFX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an AMFX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human AMFX protein. Homologous nucleic acid sequences include those nucleic acid sequences that encode conservative amino acid substitutions (see below) in SEQ ID NOS:2, 4, 6, 8, 10, 12, 14, 16, 18 and 20, as well as a polypeptide possessing AMFX biological activity. Various biological activities of the AMFX proteins are described below.

An AMFX polypeptide is encoded by the open reading frame (“ORF”) of an AMFX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human AMFX genes allows for the generation of probes and primers designed for use in identifying and/or cloning AMFX homologues in other cell types, e.g. from other tissues, as well as AMFX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS: 1, 3, 5, 7, 9, 11, 13, 15, 17 and 19; or an anti-sense strand nucleotide sequence of SEQ ID NOS: 1, 3, 5, 7, 9, 11, 13, 15, 17 and 19; or of a naturally occurring mutant of SEQ ID NOS: 1, 5, 7, 9, 11, 13, 15, 17 and 19.

Probes based on the human AMFX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an AMFX protein, such as by measuring a level of an AMFX-encoding nucleic acid in a sample of cells from a subject e.g., detecting AMFX mRNA levels or determining whether a genomic AMFX gene has been mutated or deleted.

“A polypeptide having a biologically-active portion of an AMFX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of AMFX” can be prepared by isolating a portion of SEQ ID NOS: 1, 3, 5, 7, 9, 11, 13, 15, 17 and 19, that encodes a polypeptide having an AMFX biological activity (the biological activities of the AMFX proteins are described below), expressing the encoded portion of AMFX protein (e.g., by recombinant expression in vitro) and assessing the activity of the encoded portion of AMFX.

AMFX Nucleic Acid and Polypeptide Variants

The invention further encompasses nucleic acid molecules that differ from the nucleotide sequences shown in SEQ ID NOS:1, 3, 5, 7, 9, 11, 13, 15, 17 and 19, due to degeneracy of the genetic code and thus encode the same AMFX proteins as that encoded by the nucleotide sequences shown in SEQ ID NO NOS:1, 3, 5, 7, 9, 11, 13, 15, 17 and 19. In another embodiment, an isolated nucleic acid molecule of the invention has a nucleotide sequence encoding a protein having an amino acid sequence shown in SEQ ID NOS:2, 4, 6, 8, 10, 12, 14, 16, 18 and 20.

In addition to the human AMFX nucleotide sequences shown in SEQ ID NOS:1, 3, 5, 7, 9, 11, 13, 15, 17 and 19, it will be appreciated by those skilled in the art that DNA sequence polymorphisms that lead to changes in the amino acid sequences of the AMFX polypeptides may exist within a population (e.g., the human population). Such genetic polymorphism in the AMFX genes may exist among individuals within a population due to natural allelic variation. As used herein, the terms “gene” and “recombinant gene” refer to nucleic acid molecules comprising an open reading frame (ORF) encoding an AMFX protein, preferably a vertebrate AMFX protein. Such natural allelic variations can typically result in 1-5% variance in the nucleotide sequence of the AMFX genes. Any and all such nucleotide variations and resulting amino acid polymorphisms in the AMFX polypeptides, which are the result of natural allelic variation and that do not alter the functional activity of the AMFX polypeptides, are intended to be within the scope of the invention.

Moreover, nucleic acid molecules encoding AMFX proteins from other species, and thus that have a nucleotide sequence that differs from the human sequence of SEQ ID NOS: 1, 3, 5, 7, 9, 11, 13, 15, 17 and 19, are intended to be within the scope of the invention. Nucleic acid molecules corresponding to natural allelic variants and homologues of the AMFX cDNAs of the invention can be isolated based on their homology to the human AMFX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17 and 19. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding AMFX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at

pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences of SEQ ID NOS: 1, 3, 5, 7, 9, 11, 13, 15, 17 and 19, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17 and 19, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in IX SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS: 1, 3, 5, 7, 9, 11, 13, 15, 17 and 19, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of AMFX sequences that may exist in the population, the skilled artisan will further appreciate that changes can be introduced by mutation into the nucleotide sequences of SEQ ID NO NOS:1, 3, 5, 7, 9, 11, 13, 15, 17 and 19, thereby leading to changes in the amino acid sequences of the encoded AMFX proteins, without altering the functional ability of said AMFX proteins. For example, nucleotide substitutions leading to amino acid substitutions at “non-essential” amino acid residues can be made in the sequence of SEQ ID NOS:2, 4, 6, 8, 10, 12, 14, 16, 18 and 20. A “non-essential” amino acid residue is a residue that can be altered from the wild-type sequences of the AMFX proteins without altering their biological activity, whereas an “essential” amino acid residue is required for such biological activity. For example, amino acid residues that are conserved among the AMFX proteins of the invention are predicted to be particularly non-amenable to alteration. Amino acids for which conservative substitutions can be made are well-known within the art.

Another aspect of the invention pertains to nucleic acid molecules encoding AMFX proteins that contain changes in amino acid residues that are not essential for activity. Such AMFX proteins differ in amino acid sequence from SEQ ID NOS:2, 4, 6, 8, 10, 12, 14, 16, 18 and 20, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 10, 12, 14, 16, 18 and 20. Preferably, the protein encoded by the nucleic acid molecule is at least about 60% homologous to SEQ ID NOS:2, 4, 6, 8, 10, 12, 14, 16, 18 and 20; more preferably at least about 70% homologous to SEQ ID NOS:2 , 4 , 6 , 8, 10, 12 , 14 , 16 , 18 and 20 ; still more preferably at least about 80% homologous to SEQ ID NOS:2, 4, 6, 8, 10, 12, 14, 16, 18 and 20; even more preferably at least about 90% homologous to SEQ ID NOS:2, 4, 6, 8, 10, 12, 14, 16, 18 and 20; and most preferably at least about 95% homologous to SEQ ID NOS:2, 4, 6, 8, 10, 12, 14, 16, 18 and 20.

An isolated nucleic acid molecule encoding an AMFX protein homologous to the protein of SEQ ID NOS:2, 4, 6, 8, 10, 12, 14, 16, 18 and 20, can be created by introducing one or more nucleotide substitutions, additions or deletions into the nucleotide sequence of SEQ ID NOS:1, 3, 5, 7, 9, 11, 13, 15, 17 and 19, such that one or more amino acid substitutions, additions or deletions are introduced into the encoded protein.

Mutations can be introduced into SEQ ID NOS:2, 4, 6, 8, 10, 12, 14, 16, 18 and 20, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the AMFX protein is replaced with another amino acid residue from the same side chain family. Alternatively, in another embodiment, mutations can be introduced randomly along all or part of an AMFX coding sequence, such as by saturation mutagenesis, and the resultant mutants can be screened for AMFX biological activity to identify mutants that retain activity. Following mutagenesis of SEQ ID NOS:2, 4, 6, 8, 10, 12, 14, 16, 18 and 20, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, V_{L}IM, HFY, wherein the letters within each group represent the single letter amino acid code.

In one embodiment, a mutant AMFX protein can be assayed for (i) the ability to form protein:protein interactions with other AMFX proteins, other cell-surface proteins, or biologically-active portions thereof, (ii) complex formation between a mutant AMFX protein and an AMFX ligand; or (iii) the ability of a mutant AMFX protein to bind to an intracellular target protein or biologically-active portion thereof; (e.g. avidin proteins).

In yet another embodiment, a mutant AMFX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17 and 19, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire AMFX coding strand, or to only a portion thereof. Nucleic acid molecules encoding fragments, homologs, derivatives and analogs of an AMFX protein of SEQ ID NOS:2, 4, 6, 8, 10, 12, 14, 16, 18 and 20; or antisense nucleic acids complementary to an AMFX nucleic acid sequence of SEQ ID NOS:1, 3, 5, 7, 9, 11, 13, 15, 17 and 19, are additionally provided.

In one embodiment, an antisense nucleic acid molecule is antisense to a “coding region” of the coding strand of a nucleotide sequence encoding an AMFX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the AMFX protein. The term “noncoding region” refers to 5′ and 3′ sequences which flank the coding region that are not translated into amino acids (i.e., also referred to as 5′ and 3′untranslated regions).

Given the coding strand sequences encoding the AMFX protein disclosed herein, antisense nucleic acids of the invention can be designed according to the rules of Watson and Crick or Hoogsteen base pairing. The antisense nucleic acid molecule can be complementary to the entire coding region of AMFX mRNA, but more preferably is an oligonucleotide that is antisense to only a portion of the coding or noncoding region of AMFX mRNA. For example, the antisense oligonucleotide can be complementary to the region surrounding the translation start site of AMFX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an AMFX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave AMFX mRNA transcripts to thereby inhibit translation of AMFX mRNA. A ribozyme having specificity for an AMFX-encoding nucleic acid can be designed based upon the nucleotide sequence of an AMFX cDNA disclosed herein (i.e., SEQ ID NOS:1, 3, 5, 7, 9, 11, 13, 15, 17 and 19). For example, a derivative of a Tetrahymena L-19 IVS RNA can be constructed in which the nucleotide sequence of the active site is complementary to the nucleotide sequence to be cleaved in an AMFX-encoding mRNA. See, e.g., U.S. Pat. No. 4,987,071 to Cech, et al. and U.S. Pat. No. 5,116,742 to Cech, et al. AMFX mRNA can also be used to select a catalytic RNA having a specific ribonuclease activity from a pool of RNA molecules. See, e.g., Bartel et al., (1993) Science 261:1411-1418.

Alternatively, AMFX gene expression can be inhibited by targeting nucleotide sequences complementary to the regulatory region of the AMFX nucleic acid (e.g., the AMFX promoter and/or enhancers) to form triple helical structures that prevent transcription of the AMFX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the AMFX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of AMFX can be used in therapeutic and diagnostic applications. For example, PNAs can be used as antisense or antigene agents for sequence-specific modulation of gene expression by, e.g., inducing transcription or translation arrest or inhibiting replication. PNAs of AMFX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of AMFX can be modified, e.g, to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of AMFX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, etal., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

AMFX Polypeptides

A polypeptide according to the invention includes a polypeptide including the amino acid sequence of AMFX polypeptides whose sequences are provided in SEQ ID NOS:2, 4, 6, 8, 10, 12, 14, 16, 18 and 20. The invention also includes a mutant or variant protein any of whose residues may be changed from the corresponding residues shown in SEQ ID NOS:2, 4, 6, 8, 10, 12, 14, 16, 18 and 20, while still encoding a protein that maintains its AMFX activities and physiological functions, or a functional fragment thereof.

In general, an AMFX variant that preserves AMFX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated AMFX proteins, and biologically-active portions thereof, or derivatives, fragments, analogs or homologs thereof. Also provided are polypeptide fragments suitable for use as immunogens to raise anti-AMFX antibodies. In one embodiment, native AMFX proteins can be isolated from cells or tissue sources by an appropriate purification scheme using standard protein purification techniques. In another embodiment, AMFX proteins are produced by recombinant DNA techniques. Alternative to recombinant expression, an AMFX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the AMFX protein is derived, or substantially free from chemical precursors or other chemicals when chemically synthesized. The language “substantially free of cellular material” includes preparations of AMFX proteins in which the protein is separated from cellular components of the cells from which it is isolated or recombinantly-produced. In one embodiment, the language “substantially free of cellular material” includes preparations of AMFX proteins having less than about 30% (by dry weight) of non-AMFX proteins (also referred to herein as a “contaminating protein”), more preferably less than about 20% of non-AMFX proteins, still more preferably less than about 10% of non-AMFX proteins, and most preferably less than about 5% of non-AMFX proteins. When the AMFX protein or biologically-active portion thereof is recombinantly-produced, it is also preferably substantially free of culture medium, i.e., culture medium represents less than about 20%, more preferably less than about 10%, and most preferably less than about 5% of the volume of the AMFX protein preparation.

The language “substantially free of chemical precursors or other chemicals” includes preparations of AMFX proteins in which the protein is separated from chemical precursors or other chemicals that are involved in the synthesis of the protein. In one embodiment, the language “substantially free of chemical precursors or other chemicals” includes preparations of AMFX proteins having less than about 30% (by dry weight) of chemical precursors or non-AMFX chemicals, more preferably less than about 20% chemical precursors or non-AMFX chemicals, still more preferably less than about 10% chemical precursors or non-AMFX chemicals, and most preferably less than about 5% chemical precursors or non-AMFX chemicals.

Biologically-active portions of AMFX proteins include peptides comprising amino acid sequences sufficiently homologous to or derived from the amino acid sequences of the AMFX proteins (e.g., the amino acid sequence shown in SEQ ID NOS:2, 4, 6, 8, 10, 12, 14, 16, 18 and 20) that include fewer amino acids than the full-length AMFX proteins, and exhibit at least one activity of an AMFX protein. Typically, biologically-active portions comprise a domain or motif with at least one activity of the AMFX protein. A biologically-active portion of an AMFX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native AMFX protein.

In an embodiment, the AMFX protein has an amino acid sequence shown in SEQ ID NOS:2, 4, 6, 8, 10, 12, 14, 16, 18 and 20. In other embodiments, the AMFX protein is substantially homologous to SEQ ID NOS:2, 4, 6, 8, 10, 12, 14, 16, 18 and 20, and retains the functional activity of the protein of SEQ ID NOS:2, 4, 6, 8, 10, 12, 14, 16, 18 and 20, yet differs in amino acid sequence due to natural allelic variation or mutagenesis, as described in detail, below. Accordingly, in another embodiment, the AMFX protein is a protein that comprises an amino acid sequence at least about 45% homologous to the amino acid sequence of SEQ ID NOS:2, 4, 6, 8, 10, 12, 14, 16, 18 and 20, and retains the functional activity of the AMFX proteins of SEQ ID NOS:2, 4, 6, 8, 10, 12, 14, 16, 18 and 20.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17 and 19.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides AMFX chimeric or fusion proteins. As used herein, an AMFX “chimeric protein” or “fusion protein” comprises an AMFX polypeptide operatively-linked to a non-AMFX polypeptide. An “AMFX polypeptide” refers to a polypeptide having an amino acid sequence corresponding to an AMFX protein (SEQ ID NOS:2, 4, 6, 8, 10, 12, 14, 16, 18 and 20), whereas a “non-AMFX polypeptide” refers to a polypeptide having an amino acid sequence corresponding to a protein that is not substantially homologous to the AMFX protein, e.g., a protein that is different from the AMFX protein and that is derived from the same or a different organism. Within an AMFX fusion protein the AMFX polypeptide can correspond to all or a portion of an AMFX protein. In one embodiment, an AMFX fusion protein comprises at least one biologically-active portion of an AMFX protein. In another embodiment, an AMFX fusion protein comprises at least two biologically-active portions of an AMFX protein. In yet another embodiment, an AMFX fusion protein comprises at least three biologically-active portions of an AMFX protein. Within the fusion protein, the term “operatively-linked” is intended to indicate that the AMFX polypeptide and the non-AMFX polypeptide are fused in-frame with one another. The non-AMFX polypeptide can be fused to the N-terminus or C-terminus of the AMFX polypeptide.

In one embodiment, the fusion protein is a GST-AMFX fusion protein in which the AMFX sequences are fused to the C-terminus of the GST (glutathione S-transferase) sequences. Such fusion proteins can facilitate the purification of recombinant AMFX polypeptides.

In another embodiment, the fusion protein is an AMFX protein containing a heterologous signal sequence at its N-terminus. In certain host cells (e.g., mammalian host cells), expression and/or secretion of AMFX can be increased through use of a heterologous signal sequence.

In yet another embodiment, the fusion protein is an AMFX-immunoglobulin fusion protein in which the AMFX sequences are fused to sequences derived from a member of the immunoglobulin protein family. The AMFX-immunoglobulin fusion proteins of the invention can be incorporated into pharmaceutical compositions and administered to a subject to inhibit an interaction between an AMFX ligand and an AMFX protein on the surface of a cell, to thereby suppress AMFX-mediated signal transduction in vivo. The AMFX-immunoglobulin fusion proteins can be used to affect the bioavailability of an AMFX cognate ligand. Inhibition of the AMFX ligand/AMFX interaction may be useful therapeutically for both the treatment of proliferative and differentiative disorders, as well as modulating (e.g. promoting or inhibiting) cell survival. Moreover, the AMFX-immunoglobulin fusion proteins of the invention can be used as immunogens to produce anti-AMFX antibodies in a subject, to purify AMFX ligands, and in screening assays to identify molecules that inhibit the interaction of AMFX with an AMFX ligand.

An AMFX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An AMFX-encoding nucleic acid can be cloned into such an expression vector such that the fusion moiety is linked in-frame to the AMFX protein.

AMFX Agonists and Antagonists

The invention also pertains to variants of the AMFX proteins that function as either AMFX agonists (i.e., mimetics) or as AMFX antagonists. Variants of the AMFX protein can be generated by mutagenesis (e.g., discrete point mutation or truncation of the AMFX protein). An agonist of the AMFX protein can retain substantially the same, or a subset of, the biological activities of the naturally occurring form of the AMFX protein. An antagonist of the AMFX protein can inhibit one or more of the activities of the naturally occurring form of the AMFX protein by, for example, competitively binding to a downstream or upstream member of a cellular signaling cascade which includes the AMFX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the AMFX proteins.

Variants of the AMFX proteins that function as either AMFX agonists (i.e., mimetics) or as AMFX antagonists can be identified by screening combinatorial libraries of mutants (e.g., truncation mutants) of the AMFX proteins for AMFX protein agonist or antagonist activity. In one embodiment, a variegated library of AMFX variants is generated by combinatorial mutagenesis at the nucleic acid level and is encoded by a variegated gene library. A variegated library of AMFX variants can be produced by, for example, enzymatically ligating a mixture of synthetic oligonucleotides into gene sequences such that a degenerate set of potential AMFX sequences is expressible as individual polypeptides, or alternatively, as a set of larger fusion proteins (e.g., for phage display) containing the set of AMFX sequences therein. There are a variety of methods which can be used to produce libraries of potential AMFX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential AMFX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the AMFX protein coding sequences can be used to generate a variegated population of AMFX fragments for screening and subsequent selection of variants of an AMFX protein. In one embodiment, a library of coding sequence fragments can be generated by treating a double stranded PCR fragment of an AMFX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the AMFX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of AMFX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify AMFX variants. See, e.g., Arkin and Yourvan, 1992. Proc. Natl. Acad. Sci. USA 89: 7811-7815; Delgrave, et al., 1993. Protein Engineering 6:327-331.

Anti-AMFX Antibodies

The invention encompasses antibodies and antibody fragments, such as F_{ab} or (F_{ab})₂, that bind immunospecifically to any of the AMFX polypeptides of said invention.

An isolated AMFX protein, or a portion or fragment thereof, can be used as an immunogen to generate antibodies that bind to AMFX polypeptides using standard techniques for polyclonal and monoclonal antibody preparation. The full-length AMFX proteins can be used or, alternatively, the invention provides antigenic peptide fragments of AMFX proteins for use as immunogens. The antigenic AMFX peptides comprises at least 4 amino acid residues of the amino acid sequence shown in SEQ ID NO NOS:2, 4, 6, 8, 10, 12, 14, 16, 18 and 20, and encompasses an epitope of AMFX such that an antibody raised against the peptide forms a specific immune complex with AMFX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of AMFX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, AMFX protein sequences of SEQ ID NOS:2, 4, 6, 8, 10, 12, 14, 16, 18 and 20, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as AMFX. Such antibodies include, but are not limited to, polyclonal, monoclonal, chimeric, single chain, F_{ab} and F_{(ab′)2} fragments, and an F_{ab} expression library. In a specific embodiment, antibodies to human AMFX proteins are disclosed. Various procedures known within the art may be used for the production of polyclonal or monoclonal antibodies to an AMFX protein sequence of SEQ ID NOS:2, 4, 6, 8, 10, 12, 14, 16, 18 and 20, or a derivative, fragment, analog or homolog thereof. Some of these proteins are discussed below.

Also included in the invention are antibodies to AMFX proteins, or fragments of AMFX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, F_{ab} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated AMFX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of AMFX-related protein that is located on the surface of the protein, e.g., a hydrophilic region. A hydrophobicity analysis of the human AMFX-related protein sequence will indicate which regions of a AMFX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RJA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No.5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CHI) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (Fc R), such asFc RI (CD64),Fc RII (CD32) andFc RIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g, an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diiusocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., PCT Publication WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

AMFX Recombinant Expression Vectors and Host Cells

Another aspect of the invention pertains to vectors, preferably expression vectors, containing a nucleic acid encoding an AMFX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., AMFX proteins, mutant forms of AMFX proteins, fusion proteins, etc.).

The recombinant expression vectors of the invention can be designed for expression of AMFX proteins in prokaryotic or eukaryotic cells. For example, AMFX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the AMFX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, AMFX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pV_{L} series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g, tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:

268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to AMFX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, AMFX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding AMFX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) AMFX protein. Accordingly, the invention further provides methods for producing AMFX protein using the host cells of the invention. In one embodiment, the method comprises culturing the host cell of invention (into which a recombinant expression vector encoding AMFX protein has been introduced) in a suitable medium such that AMFX protein is produced. In another embodiment, the method further comprises isolating AMFX protein from the medium or the host cell.

Transgenic AMFX Animals

The host cells of the invention can also be used to produce non-human transgenic animals. For example, in one embodiment, a host cell of the invention is a fertilized oocyte or an embryonic stem cell into which AMFX protein-coding sequences have been introduced. Such host cells can then be used to create non-human transgenic animals in which exogenous AMFX sequences have been introduced into their genome or homologous recombinant animals in which endogenous AMFX sequences have been altered. Such animals are useful for studying the function and/or activity of AMFX protein and for identifying and/or evaluating modulators of AMFX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous AMFX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing AMFX-encoding nucleic acid into the male pronuclei of a fertilized oocyte (e.g., by microinjection, retroviral infection) and allowing the oocyte to develop in a pseudopregnant female foster animal. The human AMFX cDNA sequences of SEQ ID NOS: 1, 3, 5, 7, 9, 11, 13, 15, 17 and 19, can be introduced as a transgene into the genome of a non-human animal. Alternatively, a non-human homologue of the human AMFX gene, such as a mouse AMFX gene, can be isolated based on hybridization to the human AMFX cDNA (described further supra) and used as a transgene. Intronic sequences and polyadenylation signals can also be included in the transgene to increase the efficiency of expression of the transgene. A tissue-specific regulatory sequence(s) can be operably-linked to the AMFX transgene to direct expression of AMFX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the AMFX transgene in its genome and/or expression of AMFX mRNA in tissues or cells of the animals. A transgenic founder animal can then be used to breed additional animals carrying the transgene. Moreover, transgenic animals carrying a transgene-encoding AMFX protein can further be bred to other transgenic animals carrying other transgenes.

To create a homologous recombinant animal, a vector is prepared which contains at least a portion of an AMFX gene into which a deletion, addition or substitution has been introduced to thereby alter, e.g., functionally disrupt, the AMFX gene. The AMFX gene can be a human gene (e.g., the cDNA of SEQ ID NOS:1, 3, 5, 7, 9, 11, 13,15,17 and 19), but more preferably, is a non-human homologue of a human AMFX gene. For example, a mouse homologue ofhuman AMFX gene of SEQ ID NOS:1, 3, 5,7, 9, 11, 13, 15, 17 and 19, can be used to construct a homologous recombination vector suitable for altering an endogenous AMFX gene in the mouse genome. In one embodiment, the vector is designed such that, upon homologous recombination, the endogenous AMFX gene is functionally disrupted (i.e., no longer encodes a functional protein; also referred to as a “knock out” vector).

Alternatively, the vector can be designed such that, upon homologous recombination, the endogenous AMFX gene is mutated or otherwise altered but still encodes functional protein (e.g., the upstream regulatory region can be altered to thereby alter the expression of the endogenous AMFX protein). In the homologous recombination vector, the altered portion of the AMFX gene is flanked at its 5′- and 3′-termini by additional nucleic acid of the AMFX gene to allow for homologous recombination to occur between the exogenous AMFX gene carried by the vector and an endogenous AMFX gene in an embryonic stem cell. The additional flanking AMFX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced AMFX gene has homologously-recombined with the endogenous AMFX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P 1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The AMFX nucleic acid molecules, AMFX proteins, and anti-AMFX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an AMFX protein or anti-AMFX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express AMFX protein (e.g., via a recombinant expression vector in a host cell in gene therapy applications), to detect AMFX mRNA (e.g., in a biological sample) or a genetic lesion in an AMFX gene, and to modulate AMFX activity, as described further, below. In addition, the AMFX proteins can be used to screen drugs or compounds that modulate the AMFX protein activity or expression as well as to treat disorders characterized by insufficient or excessive production of AMFX protein or production of AMFX protein forms that have decreased or aberrant activity compared to AMFX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-AMFX antibodies of the invention can be used to detect and isolate AMFX proteins and modulate AMFX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to AMFX proteins or have a stimulatory or inhibitory effect on, e.g., AMFX protein expression or AMFX protein activity. The invention also includes compounds identified in the screening assays described herein.

In one embodiment, the invention provides assays for screening candidate or test compounds which bind to or modulate the activity of the membrane-bound form of an AMFX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U S.A. 90: 6909; Erb, et al., 1994. Proc. Natl. Acad. Sci. U.S.A. 91: 11422; Zuckermann, et al., 1994. J Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of AMFX protein, or a biologically-active portion thereof, on the cell surface is contacted with a test compound and the ability of the test compound to bind to an AMFX protein determined. The cell, for example, can of mammalian origin or a yeast cell. Determining the ability of the test compound to bind to the AMFX protein can be accomplished, for example, by coupling the test compound with a radioisotope or enzymatic label such that binding of the test compound to the AMFX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of AMFX protein, or a biologically-active portion thereof, on the cell surface with a known compound which binds AMFX to form an assay mixture, contacting the assay mixture with a test compound, and determining the ability of the test compound to interact with an AMFX protein, wherein determining the ability of the test compound to interact with an AMFX protein comprises determining the ability of the test compound to preferentially bind to AMFX protein or a biologically-active portion thereof as compared to the known compound.

In another embodiment, an assay is a cell-based assay comprising contacting a cell expressing a membrane-bound form of AMFX protein, or a biologically-active portion thereof, on the cell surface with a test compound and determining the ability of the test compound to modulate (e.g., stimulate or inhibit) the activity of the AMFX protein or biologically-active portion thereof. Determining the ability of the test compound to modulate the activity of AMFX or a biologically-active portion thereof can be accomplished, for example, by determining the ability of the AMFX protein to bind to or interact with an AMFX target molecule. As used herein, a “target molecule” is a molecule with which an AMFX protein binds or interacts in nature, for example, a molecule on the surface of a cell which expresses an AMFX interacting protein, a molecule on the surface of a second cell, a molecule in the extracellular milieu, a molecule associated with the internal surface of a cell membrane or a cytoplasmic molecule. An AMFX target molecule can be a non-AMFX molecule or an AMFX protein or polypeptide of the invention. In one embodiment, an AMFX target molecule is a component of a signal transduction pathway that facilitates transduction of an extracellular signal (e.g. a signal generated by binding of a compound to a membrane-bound AMFX molecule) through the cell membrane and into the cell. The target, for example, can be a second intercellular protein that has catalytic activity or a protein that facilitates the association of downstream signaling molecules with AMFX.

Determining the ability of the AMFX protein to bind to or interact with an AMFX target molecule can be accomplished by one of the methods described above for determining direct binding. In one embodiment, determining the ability of the AMFX protein to bind to or interact with an AMFX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an AMFX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an AMFX protein or biologically-active portion thereof with a test compound and determining the ability of the test compound to bind to the AMFX protein or biologically-active portion thereof. Binding of the test compound to the AMFX protein can be determined either directly or indirectly as described above. In one such embodiment, the assay comprises contacting the AMFX protein or biologically-active portion thereof with a known compound which binds AMFX to form an assay mixture, contacting the assay mixture with a test compound, and determining the ability of the test compound to interact with an AMFX protein, wherein determining the ability of the test compound to interact with an AMFX protein comprises determining the ability of the test compound to preferentially bind to AMFX or biologically-active portion thereof as compared to the known compound.

In still another embodiment, an assay is a cell-free assay comprising contacting AMFX protein or biologically-active portion thereof with a test compound and determining the ability of the test compound to modulate (e.g. stimulate or inhibit) the activity of the AMFX protein or biologically-active portion thereof. Determining the ability of the test compound to modulate the activity of AMFX can be accomplished, for example, by determining the ability of the AMFX protein to bind to an AMFX target molecule by one of the methods described above for determining direct binding. In an alternative embodiment, determining the ability of the test compound to modulate the activity of AMFX protein can be accomplished by determining the ability of the AMFX protein further modulate an AMFX target molecule. For example, the catalytic/enzymatic activity of the target molecule on an appropriate substrate can be determined as described, supra.

In yet another embodiment, the cell-free assay comprises contacting the AMFX protein or biologically-active portion thereof with a known compound which binds AMFX protein to form an assay mixture, contacting the assay mixture with a test compound, and determining the ability of the test compound to interact with an AMFX protein, wherein determining the ability of the test compound to interact with an AMFX protein comprises determining the ability of the AMFX protein to preferentially bind to or modulate the activity of an AMFX target molecule.

The cell-free assays of the invention are amenable to use of both the soluble form or the membrane-bound form of AMFX protein. In the case of cell-free assays comprising the membrane-bound form of AMFX protein, it may be desirable to utilize a solubilizing agent such that the membrane-bound form of AMFX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either AMFX protein or its target molecule to facilitate separation of complexed from uncomplexed forms of one or both of the proteins, as well as to accommodate automation of the assay. Binding of a test compound to AMFX protein, or interaction of AMFX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-AMFX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or AMFX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of AMFX protein binding or activity determined using standard techniques.

Other techniques for immobilizing proteins on matrices can also be used in the screening assays of the invention. For example, either the AMFX protein or its target molecule can be immobilized utilizing conjugation of biotin and streptavidin. Biotinylated AMFX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with AMFX protein or target molecules, but which do not interfere with binding of the AMFX protein to its target molecule, can be derivatized to the wells of the plate, and unbound target or AMFX protein trapped in the wells by antibody conjugation. Methods for detecting such complexes, in addition to those described above for the GST-immobilized complexes, include immunodetection of complexes using antibodies reactive with the AMFX protein or target molecule, as well as enzyme-linked assays that rely on detecting an enzymatic activity associated with the AMFX protein or target molecule.

In another embodiment, modulators of AMFX protein expression are identified in a method wherein a cell is contacted with a candidate compound and the expression of AMFX mRNA or protein in the cell is determined. The level of expression of AMFX mRNA or protein in the presence of the candidate compound is compared to the level of expression of AMFX mRNA or protein in the absence of the candidate compound. The candidate compound can then be identified as a modulator of AMFX mRNA or protein expression based upon this comparison. For example, when expression of AMFX mRNA or protein is greater (i.e., statistically significantly greater) in the presence of the candidate compound than in its absence, the candidate compound is identified as a stimulator of AMFX mRNA or protein expression. Alternatively, when expression of AMFX mRNA or protein is less (statistically significantly less) in the presence of the candidate compound than in its absence, the candidate compound is identified as an inhibitor of AMFX mRNA or protein expression. The level of AMFX mRNA or protein expression in the cells can be determined by methods described herein for detecting AMFX mRNA or protein.

In yet another aspect of the invention, the AMFX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with AMFX (“AMFX-binding proteins” or “AMFX-bp”) and modulate AMFX activity. Such AMFX-binding proteins are also likely to be involved in the propagation of signals by the AMFX proteins as, for example, upstream or downstream elements of the AMFX pathway.

The two-hybrid system is based on the modular nature of most transcription factors, which consist of separable DNA-binding and activation domains. Briefly, the assay utilizes two different DNA constructs. In one construct, the gene that codes for AMFX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an AMFX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with AMFX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the AMFX sequences, SEQ ID NOS:1, 3, 5, 7, 9, 11, 13, 15, 17 and 19, or fragments or derivatives thereof, can be used to map the location of the AMFX genes, respectively, on a chromosome. The mapping of the AMFX sequences to chromosomes is an important first step in correlating these sequences with genes associated with disease.

Briefly, AMFX genes can be mapped to chromosomes by preparing PCR primers (preferably 15-25 bp in length) from the AMFX sequences. Computer analysis of the AMFX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the AMFX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the AMFX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the AMFX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The AMFX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the AMFX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The AMFX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17 and 19, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining AMFX protein and/or nucleic acid expression as well as AMFX activity, in the context of a biological sample (e.g., blood, serum, cells, tissue) to thereby determine whether an individual is afflicted with a disease or disorder, or is at risk of developing a disorder, associated with aberrant AMFX expression or activity. The disorders include e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli.wasting disorders associated with chronic diseases and various cancers. The invention also provides for prognostic (or predictive) assays for determining whether an individual is at risk of developing a disorder associated with AMFX protein, nucleic acid expression or activity. For example, mutations in an AMFX gene can be assayed in a biological sample. Such assays can be used for prognostic or predictive purpose to thereby prophylactically treat an individual prior to the onset of a disorder characterized by or associated with AMFX protein, nucleic acid expression, or biological activity.

Another aspect of the invention provides methods for determining AMFX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.).

Yet another aspect of the invention pertains to monitoring the influence of agents (e.g., drugs, compounds) on the expression or activity of AMFX in clinical trials.

These and other agents are described in further detail in the following sections.

Diagnostic Assays

An exemplary method for detecting the presence or absence of AMFX in a biological sample involves obtaining a biological sample from a test subject and contacting the biological sample with a compound or an agent capable of detecting AMFX protein or nucleic acid (e.g., mRNA, genomic DNA) that encodes AMFX protein such that the presence of AMFX is detected in the biological sample. An agent for detecting AMFX mRNA or genomic DNA is a labeled nucleic acid probe capable of hybridizing to AMFX mRNA or genomic DNA. The nucleic acid probe can be, for example, a full-length AMFX nucleic acid, such as the nucleic acid of SEQ ID NOS: 1, 3, 5, 7, 9, 11, 13, 15, 17 and 19, or a portion thereof, such as an oligonucleotide of at least 15, 30, 50, 100, 250 or 500 nucleotides in length and sufficient to specifically hybridize under stringent conditions to AMFX mRNA or genomic DNA. Other suitable probes for use in the diagnostic assays of the invention are described herein.

An agent for detecting AMFX protein is an antibody capable of binding to AMFX protein, preferably an antibody with a detectable label. Antibodies can be polyclonal, or more preferably, monoclonal. An intact antibody, or a fragment thereof (e.g., F_{ab} or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect AMFX mRNA, protein, or genomic DNA in a biological sample in vitro as well as in vivo. For example, in vitro techniques for detection of AMFX mRNA include Northern hybridizations and in situ hybridizations. In vitro techniques for detection of AMFX protein include enzyme linked immunosorbent assays (ELISAs), Western blots, immunoprecipitations, and immunofluorescence. In vitro techniques for detection of AMFX genomic DNA include Southern hybridizations. Furthermore, in vivo techniques for detection of AMFX protein include introducing into a subject a labeled anti-AMFX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting AMFX protein, mRNA, or genomic DNA, such that the presence of AMFX protein, mRNA or genomic DNA is detected in the biological sample, and comparing the presence of AMFX protein, mRNA or genomic DNA in the control sample with the presence of AMFX protein, mRNA or genomic DNA in the test sample.

The invention also encompasses kits for detecting the presence of AMFX in a biological sample. For example, the kit can comprise: a labeled compound or agent capable of detecting AMFX protein or mRNA in a biological sample; means for determining the amount of AMFX in the sample; and means for comparing the amount of AMFX in the sample with a standard. The compound or agent can be packaged in a suitable container. The kit can further comprise instructions for using the kit to detect AMFX protein or nucleic acid.

Prognostic Assays

The diagnostic methods described herein can furthermore be utilized to identify subjects having or at risk of developing a disease or disorder associated with aberrant AMFX expression or activity. For example, the assays described herein, such as the preceding diagnostic assays or the following assays, can be utilized to identify a subject having or at risk of developing a disorder associated with AMFX protein, nucleic acid expression or activity. Alternatively, the prognostic assays can be utilized to identify a subject having or at risk for developing a disease or disorder. Thus, the invention provides a method for identifying a disease or disorder associated with aberrant AMFX expression or activity in which a test sample is obtained from a subject and AMFX protein or nucleic acid (e.g., mRNA, genomic DNA) is detected, wherein the presence of AMFX protein or nucleic acid is diagnostic for a subject having or at risk of developing a disease or disorder associated with aberrant AMFX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant AMFX expression or activity. For example, such methods can be used to determine whether a subject can be effectively treated with an agent for a disorder. Thus, the invention provides methods for determining whether a subject can be effectively treated with an agent for a disorder associated with aberrant AMFX expression or activity in which a test sample is obtained and AMFX protein or nucleic acid is detected (e.g., wherein the presence of AMFX protein or nucleic acid is diagnostic for a subject that can be administered the agent to treat a disorder associated with aberrant AMFX expression or activity).

The methods of the invention can also be used to detect genetic lesions in an AMFX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an AMFX-protein, or the misexpression of the AMFX gene. For example, such genetic lesions can be detected by ascertaining the existence of at least one of: (i) a deletion of one or more nucleotides from an AMFX gene; (ii) an addition of one or more nucleotides to an AMFX gene; (iii) a substitution of one or more nucleotides of an AMFX gene, (iv) a chromosomal rearrangement of an AMFX gene; (v) an alteration in the level of a messenger RNA transcript of an AMFX gene, (vi) aberrant modification of an AMFX gene, such as of the methylation pattern of the genomic DNA, (vii) the presence of a non-wild-type splicing pattern of a messenger RNA transcript of an AMFX gene, (viii) a non-wild-type level of an AMFX protein, (ix) allelic loss of an AMFX gene, and (x) inappropriate post-translational modification of an AMFX protein. As described herein, there are a large number of assay techniques known in the art which can be used for detecting lesions in an AMFX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the AMFX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an AMFX gene under conditions such that hybridization and amplification of the AMFX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an AMFX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in AMFX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in AMFX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the AMFX gene and detect mutations by comparing the sequence of the sample AMFX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the AMFX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type AMFX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in AMFX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an AMFX sequence, e.g., a wild-type AMFX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in AMFX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control AMFX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an AMFX gene.

Furthermore, any cell type or tissue, preferably peripheral blood leukocytes, in which AMFX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on AMFX activity (e.g., AMFX gene expression), as identified by a screening assay described herein can be administered to individuals to treat (prophylactically or therapeutically) disorders (The disorders include, e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli, and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of AMFX protein, expression of AMFX nucleic acid, or mutation content of AMFX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol, 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of AMFX protein, expression of AMFX nucleic acid, or mutation content of AMFX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an AMFX modulator, such as a modulator identified by one of the exemplary screening assays described herein.

Monitoring of Effects During Clinical Trials

Monitoring the influence of agents (e.g., drugs, compounds) on the expression or activity of AMFX (e.g., the ability to modulate aberrant cell proliferation and/or differentiation) can be applied not only in basic drug screening, but also in clinical trials. For example, the effectiveness of an agent determined by a screening assay as described herein to increase AMFX gene expression, protein levels, or upregulate AMFX activity, can be monitored in clinical trails of subjects exhibiting decreased AMFX gene expression, protein levels, or downregulated AMFX activity. Alternatively, the effectiveness of an agent determined by a screening assay to decrease AMFX gene expression, protein levels, or downregulate AMFX activity, can be monitored in clinical trails of subjects exhibiting increased AMFX gene expression, protein levels, or upregulated AMFX activity. In such clinical trials, the expression or activity of AMFX and, preferably, other genes that have been implicated in, for example, a cellular proliferation or immune disorder can be used as a “read out” or markers of the immune responsiveness of a particular cell.

By way of example, and not of limitation, genes, including AMFX, that are modulated in cells by treatment with an agent (e.g., compound, drug or small molecule) that modulates AMFX activity (e.g., identified in a screening assay as described herein) can be identified. Thus, to study the effect of agents on cellular proliferation disorders, for example, in a clinical trial, cells can be isolated and RNA prepared and analyzed for the levels of expression of AMFX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of AMFX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an AMFX protein, mRNA, or genomic DNA in the preadministration sample; (iii) obtaining one or more post-administration samples from the subject; (iv) detecting the level of expression or activity of the AMFX protein, mRNA, or genomic DNA in the post-administration samples; (v) comparing the level of expression or activity of the AMFX protein, mRNA, or genomic DNA in the pre-administration sample with the AMFX protein, mRNA, or genomic DNA in the post administration sample or samples; and (vi) altering the administration of the agent to the subject accordingly. For example, increased administration of the agent may be desirable to increase the expression or activity of AMFX to higher levels than detected, i.e., to increase the effectiveness of the agent. Alternatively, decreased administration of the agent may be desirable to decrease expression or activity of AMFX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant AMFX expression or activity. The disorders include, e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244:1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant AMFX expression or activity, by administering to the subject an agent that modulates AMFX expression or at least one AMFX activity. Subjects at risk for a disease that is caused or contributed to by aberrant AMFX expression or activity can be identified by, for example, any or a combination of diagnostic or prognostic assays as described herein. Administration of a prophylactic agent can occur prior to the manifestation of symptoms characteristic of the AMFX aberrancy, such that a disease or disorder is prevented or, alternatively, delayed in its progression. Depending upon the type of AMFX aberrancy, for example, an AMFX agonist or AMFX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating AMFX expression or activity for therapeutic purposes. The modulatory method of the invention involves contacting a cell with an agent that modulates one or more of the activities of AMFX protein activity associated with the cell. An agent that modulates AMFX protein activity can be an agent as described herein, such as a nucleic acid or a protein, a naturally-occurring cognate ligand of an AMFX protein, a peptide, an AMFX peptidomimetic, or other small molecule. In one embodiment, the agent stimulates one or more AMFX protein activity. Examples of such stimulatory agents include active AMFX protein and a nucleic acid molecule encoding AMFX that has been introduced into the cell. In another embodiment, the agent inhibits one or more AMFX protein activity. Examples of such inhibitory agents include antisense AMFX nucleic acid molecules and anti-AMFX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an AMFX protein or nucleic acid molecule. In one embodiment, the method involves administering an agent (e.g., an agent identified by a screening assay described herein), or combination of agents that modulates (e.g., up-regulates or down-regulates) AMFX expression or activity. In another embodiment, the method involves administering an AMFX protein or nucleic acid molecule as therapy to compensate for reduced or aberrant AMFX expression or activity.

Stimulation of AMFX activity is desirable in situations in which AMFX is abnormally downregulated and/or in which increased AMFX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The AMFX nucleic acids and proteins of the invention are useful in potential prophylactic and therapeutic applications implicated in a variety of disorders including, e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli, disorders associated with chronic diseases and various cancers.

As an example, a cDNA encoding the AMFX protein of the invention may be useful in gene therapy, and the protein may be useful when administered to a subject in need thereof. By way of non-limiting example, the compositions of the invention will have efficacy for treatment of patients suffering from: e.g., disorders related to cell signal processing, cell adhesion or migration pathway modulation, for example, but not limited to, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, cellular responses to external stimuli.

Both the novel nucleic acid encoding the AMFX protein, and the AMFX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

The following examples illustrate by way of non-limiting example various aspects of the invention.

### Example 1

### Quantitative Expression Analysis of AMF-1-10 in Various Cells and Tissues

The quantitative expression patterns of clones AMF-1-10 were assessed in a large number of normal and tumor sample cells and cell lines by real time quantitative PCR (TaqMan®) performed on a Perkin-Elmer Biosystems ABI PRISM® 7700 Sequence Detection System.

First, 96 RNA samples were normalized to β-actin and GAPDH. RNA (˜50 ng total or ˜1 ng polyA+) was converted to cDNA using the TaqMan® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TaqMan® reaction using β-actin and GAPDH TaqMan® Assay Reagents (PE Biosystems; Catalog Nos. 4310881E and 4310884E, respectively) and TaqMan® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TaqMan® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions:

Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 950 C 10 min, then 40 cycles of 95° C. for 15 seconds, 60° C. for 1 minute.

AMF-1

The nucleotide sequence used for TaqMan analysis on AMF-1 is indicated in Table 12. The oligonucleotide sequences used as primers are boxed and the oligonucleotide sequence used as a probe is underlined.

TABLE 12
AMF-1 (1429510) Sequence Input for TaqMan Analysis

| | |
|---|---|
| (reverse strand of SEQ ID NO.1): | |
| CGGATGACTCCCGAGAAGGTGAGCCCCTCACCCACATGCTAAGAGCCCCTTCTGGGCCACCCAGATCCATCTCCGC | (SEQ ID NO.21) |
| ACTGCCTGGGTCTCTGAGTTTCAGGCTCCCCCTGAGAGCCTGGGTGGCCCTGGACCCTGCCAGCCTGGGGGGCTTGGG | |
| CTTTTGTCCCCTTGGGGCCTTGAGTGTGGCCAGGGCTCTGGCGATTGTGTGGTGACAGAAGCCATGTCTGCAACGC | |
| CTGCCATCCGCAGACGTGAATGAGTGTGCAGAGAACCCTGGCGTCTGCACTAACGGCGTCTGTGTCAACACCGATG | |
| GATCCTTCCGCTGTGAGTGTCCCTTTGGCTACAGCCTGGACTTCACTGGCATCAACTGTGTGGACACAGACGAGTG | |
| CTCTGTCGGCCACCCCTGTGGGCAAGGGACATGC\overline{\|ACCAATGTCATCGGAGGCTT\|}CGAATGTGCCTGTGCTGACGGC | |
| TTTGAGCCTGGCCTC\overline{\|ATGATGACCTGCGAGGACATC\|}GACGAATGCTCCCTGAACCCGOCTGCTCTGTGCCTTCCGCT | |
| GCCACAATACCGAGGGCTCCTACCTGTGCACCTGTCCAGCCGGCTACACCCTGCGGGAGGACGGGGCCATGTGTCG | |
| AGATGTGGACGAGTGTGCAGATGGTCAGCAGGACTGCCACGCCCGGGGCATGGAGTGCAAGAACCTCATCGGTACC | |
| TTCGCGTGCGTCTGTCCCCCAGGCATGCGGCCCCTGCCTGGCTCTGGGGAGGGCTGCACAGATGACAATGAATGCC | |
| ACGCTCAGCCTGACCTCTGTGTCAACGGCCGCTGTGTCAACACCGCGGGCAGCTTCCGGTGCGACTGTGATGAGGG | |
| ATTCCAGCCCAGCCCCACCCTTACCGAGTGCCACGACATCCGOGCAGGGGCCCTGCTTTGCCGAGGTGCTGCAGACC | |
| ATGTGCCGGTCTCTGTCCAGCAGCAGTGAGGCTGTCACCAGGGCCGAGTGCTGCTGTGGGGGTGGCCGGGGCTGGG | |
| GGCCCCGCTGCGAGCTCTGTCCCCTGCCCGGCACCTCTGCCTACAGGAAGCTGTGCCCCCATGGCTCAGGCTACAC | |
| TGCTGAGGGCCGAGATGTAGATGAATGCCGTATGCTTGCTCACCTGTGTGCTCATGGGGAGTGCATCAACAGCCTT | |
| GGCTCCTTCCGCTGCCACTGTCAGGCCGGGTACACACCGGATGCTACTGCTACTACCTGCCTGGATATGGATGAGT | |
| GCAGCCAGGTCCCCAAGCCATGTACCTTCCTCTGCAAAAACACGAAGGGCAGTTTCCTGTGCAGCTGTCCCCGAGG | |
| CTACCTGCTGGAGGAGGATGGCAGGACCTGCAAAGACCTGGACGAATGCACCTCCCGGCAGCACAACTGTCAGTTC | |
| CTCTGTGTCAACACTGTGGGCGCCTTCACCTGCCGCTGTCCACCCGGCTTCACCCAGCACCACCAGGCCTGCTTCG | |
| ACAATGATGAGTGCTCAGCCCAGCCTGGCCCATGTGGTGCCCACGGGCACTGCCACAACACCCCGGGCAGCTTCCG | |
| CTGTGAATGCCACCAAGGCTTCACCCTGGTCAGCTCAGGCCATGGCTGTGAAGATGTGAATGAATGTGATGGGCCC | |
| CACCGCTGCCAGCATGGCTGTCAGAACCAGCTAGGGGGCTACCGCTGCAGCTGCCCCCAGGGTTTCACCCAGCACT | |
| CCCAGTGGGCCCAGTGTGTGGGTGAGTGAAAAGGGCTGGGAAGAAGCTGGGCCCTCCACCAGAATCTGCTCAGAGC | |
| AGGCGACTAACAGACGCCACCCTGCAAGATGATGTGACAAGCACAATTATCTAAAGATTGAACAGGCCAGCCCAGA | |
| AGATGAGAATGAGTGTGCCCTGTCGCCC | |

The following primer and probe sequences were used for TaqMan analysis of AMF-1.

| | | |
|---|---|---|
| Ag 390 (F): | 5′-ACCAATGTCATCGGAGGCTT-3′ | (SEQ ID NO.22) |
| Ag 390 (R): | 5′-GATGTCCTCGCAGGTCATCAT-3′ | (SEQ ID NO.23) |
| Ag 390 (P): | FAM-5′-TCAAAGCCGTCAGCACAGGCACA-3′-TAMRA | (SEQ ID NO.24) |

AMF-2

The nucleotide sequence used for TaqMan analysis on AMF-2 is indicated in Table 13. The oligonucleotide sequences used as primers are boxed and the oligonucleotide sequence used as a probe is underlined.

TABLE 13
AMF-2 (20421338) Sequence Input for TaqMan Analysis

| | |
|---|---|
| (reverse strand of SEQ ID NO. 3): | |
| GGAGGGCCTGTGATTCTACTGCAGGCAGGCACCCCCCACAACCTCACATGCCGGGCCTTCAATG | (SEQ ID NO. 25) |
| CGAAGCCTGCTGCCACCATCATCTGGTTCCGGGACGGGACGCAGCAGGAGGGCGCTGTGGCCAG | |
| CACGGAATTGCTGAAGGATGGGAAGAGGGAGACCACCGTGAGCCAACTGCTTATTAACCCCACG | |
| G\overline{\|ACCTGGACATAGGGCGTGTCT\|}TCACTTGCCGAAGCATGAACGAAGCCATCCCTAGT\overline{\|GGCAAGG\|} | |
| \overline{\|AGACTTCCATCGA\|}GCTGGATGTGCACCACCCTCCTACAGTGACCCTGTCCATTGAGCCACAGAC | |
| GGGGCAGGAGGGTGAGCGTGTTGTCTTTACCTGCCAGGCCACAGCCAACCCCGAGATCT | |

The following primer and probe sequences were used for TaqMan analysis of AMF-2.

| | | | |
|---|---|---|---|
| Ag 271 | (F): | 5′-ACCTGGACATAGGGCGTGTCT-3 | (SEQ ID NO. 26) |
| Ag 271 | (R): | 5′-TCGATGGAAGTCTCCTTGCC-3′ | (SEQ ID NO. 27) |
| Ag 271 | (P): | FAM-5′-CGAAGCATGAACGAAGCCATCCCTAG-3′-TAMRA | (SEQ ID NO. 28) |

AMF-3

The nucleotide sequence used for TaqMan analysis on AMF-3 is indicated in Table 14. The oligonucleotide sequences used as primers are boxed and the oligonucleotide sequence used as a probe is underlined.

TABLE 14
AMF-3 (27251385) Sequence Input for TaqMan Analysis

| | |
|---|---|
| (reverse strand of SEQ ID NO. 5): | |
| TCCAATCTCACATGCACGCACAGCCGGCCTGAGGCGTCCAGCATCAGGCCCTCTGGACACTCACAG\overline{\|CGGAAAGACC\|} | (SEQ ID NO. 29) |
| \overline{\|CAGCAGTGTT\|}GACGCAACGCCCGTTGGGACAGACTCCCGGGAAGG\overline{\|ACTCACACTCGTTCACAT\|}CATCGCAGGTGAC | |
| ACCCGTCATCCGGGCAAAGCCCCGGGCACAGGCAG\overline{GGTCGATCTCGCAGCGTTCGCAGG}GGCTCCCCCAGGCTGCC | |
| CCGAGG | |

The following primer and probe sequences were used for TaqMan analysis of AMF-3.

| | |
|---|---|
| Ag 72 | |
| F CGGAAAGACCCAGCAGTGTT | (SEQ ID NO. 30) |
| R ATGATGTGAACGAGTGTGAGTCCTT | (SEQ ID NO. 31) |
| P Fam-CGCCCGTTGGGACAGACTCCC-Tamra | (SEQ ID NO. 32) |

AMF-4

The nucleotide sequence used for TaqMan analysis on AMF-4 is indicated in Table 15. The oligonucleotide sequences used as primers are boxed and the oligonucleotide sequence used as a probe is underlined.

TABLE 15
AMF-4 (27486474) Sequence Input for TaqMan Analysis.

| | |
|---|---|
| TCACGGGAATAAGCCTGGGCCCGTCCCTTTGA\overline{\|TTTCCAACAAGATCTGCAACCA\|}CAGGGA | (SEQ ID NO. 33) |
| CGTGTACGGTGGCATCATCTCCCCCTCCATG\overline{\|CTCTGCGCGGGCTACCT\|}GACGGGTGGCGT | |
| GGACAGCTGCCAGGGGGACAGCGGGGGGCCCCTGGTGTGTCAAGAGAGGAGGCTGTGGAA | |
| GTTAGTGGGAGCGACCAGCTTTGGCATCGGCTGCGCAGAGGTGAACAAGCCTGGGGTGTA | |
| CACCGTGTCACCTCCTTCCTGGACTGGATCCACGAGCAGATGGAGAGAGACCTAAAAACC | |
| TGAAGAGGAAGGGGATAAGTAGCCACCTGAGTTCCTGAGGTGATGAAGACAGCCCGATCC | |
| TCCCCTGGACTCCCGTGTAGGAACCTGCACACGAGCAGACACCCTTGGAGCTCTGAGTTC | |
| CGGCACCAGTAGCAGGCCC | |

The following primer and probe sequences were used for TaqMan
analysis of AMF-4.

| | | | |
|---|---|---|---|
| Ag 248 | (F) : | 5′-TTTCCAACAAGATCTGCAACCA-3′ | (SEQ ID NO. 34) |
| Ag 248 | (R) : | 5′-AGGTAGCCCGCGCAGAG-3′ | (SEQ ID NO. 35) |
| Ag 248 | (P) : | FAM-5′-CGTGTACGGTGGCATCATCTCCCC-3′-TAMRA | (SEQ ID NO. 36) |

The nucleotide sequence used for TaqMan analysis on AMF-5 is indicated in Table 16. The oligonucleotide sequences used as primers are boxed and the oligonucleotide sequence used as a probe is underlined.

TABLE 16
AMF-5 (29691387) Sequence Input for TaqMan Analysis

| | |
|---|---|
| TGTCATTGTCCTTTTACCTATTATATTTTTTCATACTCTGTGAAAACAAATCAGTTGCCGGACTAACCATGACCTATGATGGAA | (SEQ ID NO. 37) |
| ATAATCCAGTGACATCTCATAGAGATGTGCCACTTTCTTATTGC\overline{\|AACTCAGACTGCAATTGTGATGAAA\|}GTCAGTGGGAACCAG | |
| TCTGTGGGAACAAT\overline{\|GGAATAACTTACCTGTCACCTTGTCTAG\|}CAGGATGCAAATCCTCAAGTGGTATTAAAAAGCATACAGTGT | |
| TTTATAACTGTAGTTGTGTGGAAGTAACTGGTCTCCAGAACAGAAATTACTCAGCGCACTTGGGTGAATGCCCAAGAGATAATA | |
| CTTGTACAAGGAAATTTTTCATCTATGTTGCAATTCAAGTCATAAACTCTTTGTTCTCTGCAACAGGAGGTACC | |

The following primer and probe sequences were used for TaqMan analysis of AMF-5.

| | | | |
|---|---|---|---|
| Ag 287 | (F): | 5′-AACTCAGACTGCAATTGTGATGAAA-3′ | (SEQ ID NO. 38) |
| Ag 287 | (R): | 5′-CTAGACAAGGTGACAGGTAAGTTATTCC-3′ | (SEQ ID NO. 39) |
| Ag 287 | (P): | TET-5′-TTGTTCCCACAGACTGGTTCCCACTGT-3′-TAMRA | (SEQ ID NO. 40) |

AMF-6

The nucleotide sequence used for TaqMan analysis on AMF-6 is indicated in Table 17. The oligonucleotide sequences used as primers are boxed and the oligonucleotide sequence used as a probe is underlined.

TABLE 17
AMF-6 (38905521) Sequence Input for TaqMan Analysis

| | |
|---|---|
| TGGCAGCCCTGGAGGAGCCGATGGTGGACCTGGACGGCGAGCTGCCTTTCGTGCGGCCCCTGCCCCACATTGCCGT | (SEQ ID NO. 41) |
| GCTCCAGGAC\overline{\|GAGCTGCCGCAACTCTTCC\|}AGGATGACGACGTCGGGGCCGATGAGGAAGAGGCAGAGTTGCGGGGC | |
| GAA\overline{\|CACACGCTCACAGAGAAGTTTGTC\|}TGCCTGGATGACTCCTTTGGCCATGACTGCAGCTTGACCTGTGATGACT | |
| GCAGGAACGGAGGGACCTGCCTCCTGGGCCTGGATGGCTGTGATTGCCCCGAGGGGTGGACTGGGGTTATTTGCAA | |
| TGAGATTTGTCCTCCGGA | |

The following primer and probe sequences were used for TaqMan analysis of AMF-6.

Ag 252 (F): 5′-GAGCTGCCGCAACTCTTCC-3′ (SEQ ID NO. 42)

Ag 252 (R): 5′-GACAAACTTCTCTGTGAGCGTGTG-3′ (SEQ ID NO. 43)

Ag 252 (P): TET-5′-CGCAACTCTGCCTCTTCCTCATCGG-3′-TAMRA (SEQ ID NO. 44)

AMF-7

The nucleotide sequence used for TaqMan analysis on AMF-7 is indicated in Table 18. The oligonucleotide sequences used as primers are boxed and the oligonucleotide sequence used as a probe is underlined.

TABLE 18
AMF-7 (4194093) Sequence Input for TaqMan Analysis

| | |
|---|---|
| (reverse strand of SEQ ID NO. 13): | |
| cgccttcatgctgccggcgggctgctcgcgccggctggtggccgagctgcagggcgccct | (SEQ ID NO. 45) |
| ggacgcctgcgcacagcgacaattgcaattggagcagagcctgcgcgtttgccgtcggct | |
| gctgcatgcctgggaaccaactgggacccgggctttgaagccacctccagggccagaaac | |
| taatggagaggacccccttccagcatgcacacccagtccacaagacctcaaagagttgga | |
| gtttctgacccaggcactggagaaggctgtacgagttcgaagaggcatcactaaggccga | |
| agagagagacaaggcccccagcctgaaatctaggtccattgtcacctcttctggcacgac | |
| agcctccgccccaccgcattccccaggccaagctggtggccatgcttcagacacgagacc | |
| caccaagggcctccgccagaccacggtgcctgccaagggccaccctgagcgccggctgct | |
| gtcagtgggggatgggacccgtgttgggatgggagcccgaacccccaggcctggggcggg | |
| cctcagggaccagcaaatggccccatccgctgctcctcaggccccagaagccttcacact | |
| caaggagaaggggcacctgctgcggctgcctgc\overline{\|ggcattcaggaaagcagctt\|}cccagaa | |
| ctcgagcctgtgggcccagctcagttccacacag\overline{\|accagtgattccacggatgc\|}cgccgc | |
| tgccaaaacccagttcctccagaacatgcagacagcttcaggcgggccccagcccaggct | |
| cagtgctgtggaggtggaggcggaggcggggcgcctgcggaaggcctgctcgctgctgag | |
| actgcgcatgagggaggagctctcagcagcccccatggactggatgcaggagtaccgctg | |
| cctgctcacgctggaggggctgcaggccatggtgggccagtgtctgcacaggctgcagga | |
| gctgcgtgcagcggtggcggaacagccaccaagaccatgtcctgtggggaggccccccgg | |
| agcctcgccgtcctgtgggggtagagcggagcctgcatggagcccccagctgcttgtcta | |
| ctccagcacccaggagctgcagaccctggcggccctcaagctgcgagtggctgtgctgga | |
| ccagcagatccacttggaaaaggtcctgatggctgaactcctccccctggtaagcgctgc | |
| acagccgcaggggccgccctggctggccctgtgccgggctgtgcacagcctgctctgcga | |
| gggaggagcacgtgtccttaccatcctgcgggatgaacctgcagtctgagcctttcccat | |
| gctgccctcggc | |

The following primer and probe sequences were used for TaqMan analysis of AMF-7.

| | | | |
|---|---|---|---|
| Ab16 | (F): | 5′-GGCATTCAGGAAAGCAGCTT-3′ | (SEQ ID NO. 46) |
| Ab16 | (R): | 5′-GCATCCGTGGAATCACTGGT-3′ | (SEQ ID NO. 47) |
| Ab16 | (P): | FAM-5′-TGGGCCCAGCTCAGTTCCACACA-TAMRA | (SEQ ID NO. 48) |

AMF-8

The nucleotide sequence used for TaqMan analysis on AMF-8 is indicated in Table 19. The oligonucleotide sequences used as primers are boxed and the oligonucleotide sequence used as a probe is underlined.

TABLE 19
AMF-8 (AC011036 A) Sequence Input for TaqMan Analysis

| | |
|---|---|
| (reverse strand of SEQ ID NO.15) | |
| ATGCAGGCTCAACAGTACCAGCAGCAGCGTCGAAAATTTGCAGCTGCCTTCTTGGCATTCATTTTCATACTGGCAG | (SEQ ID NO.52) |
| CTGTGGATACTGCTGAAGCAGGGAAGAAAGAGAAACCAGAAAAAAAAGTGAAGAAGTCTGACTGTGGAGAATGGCA | |
| GTGGAGTGTGTGTGTGCCCACCAGTGGAGACTGTGGGCTGGGCACACGGGAGGGCACTCGGACTGGAGCTGAGTGC | |
| AAGCAAACCATGAAGACCCAGAGATGTAAGATCCCCTGCAACTGGAAGAAGCAATTTGGCGCGGAGTGCAAATACC | |
| AGTTCCAGGCCTGGGGAGAATGTGACCTGAACACAGCCCTGAAGACCAGAACTGGAAGTCTGAAGCGAG\overline{\|CCCTGCA\|} | |
| \overline{\|CAATGCCGAAT\|}GCCAGAAGACTGTCACCATCTCCAAGCCCTGTGGCAAA\overline{\|CTGACCAAGCCCAAACCTCA\|}AGGTACC | |
| CTAGAACTTAAAGTAAAAAAAAAAAAAAAAAAAAAAAATTCTGAGGAGACCTTTTAG | |

The following primer and probe sequences were used for TaqMan analysis of AMF-8.

TABLE 20
+HZ,64
AMF-9 (AL307658) Sequence Input for TaqMan Analysis

| | |
|---|---|
| TTTTTGAAGTTTTCATTCATAIAATGCATAGACAATGGGATTACAGATGGAGTTGGAAAATCCAATAATTTGCACGA | (SEQ ID NO.53) |
| TAGCAAAAATCATCTTGATTGTGACATCATCATATTCCTTTTCAAAATTACTGTATTCAATCATCATATGGACAAC | |
| \overline{\|ATGGAATGGTGCCCAGCA\|}CACAGCAAAGAGAGCCACCACTGTCACCATCATAA \overline{\|TGACAGCTCGTTTCTTCT\|}TCCAT | |
| AAGAGGCAGGAGGAAGAGGATGACAAGGATGAAGGTGGTGTAGATCTTCTGGTGCACAGGGCTGGTCCACTCTTCT | |
| AAGCAGCAGATGTGTTCCTTTTCATATAGGAAGTCATATTTGATCTCAAGTTGTTGCACGTGCCACATGGGTGATC | |
| CTACGATGACTGCCACCAGCCAGACCACACCTAGCATTGTGAAAGCCCTTCG | |

AMF-9

The nucleotide sequence used for TaqMan analysis on AMF-9 is indicated in Table 20. The oligonucleotide sequences used as primers are boxed and the oligonucleotide sequence used as a probe is underlined.

| | |
|---|---|
| GPCR 13 (F): 5′-ATGGAATGGTGCCCAGCA3′ | (SEQ ID NO.54) |
| GPCR 13 (R): 5′-TGGAAGAAGAAACGAGCTGTCA3′(SEQ ID NO.55) | |
| GPCR 13 (P): 5′-CAGCAAAGAGAGCCACCACTGTCACCA3′(SEQ ID NO.56) | |

The following primer and probe sequences were used for TaqMan analysis of AMF-9.

| | |
|---|---|
| GPCR 13 (F): 5′-ATGGAATGGTGCCCAGCA-3′ | (SEQ ID NO.54) |
| GPCR 13 (R): 5′-TGGAAGAAGAAACGAGCTGTCA-3′ | (SEQ ID NO.55) |
| GPCR 13 (P): 5′-CAGCAAAGAGAGCCACCACTGTCACCA-3′ | (SEQ ID NO.56) |

AMF-10

The nucleotide sequence used for TaqMan analysis on AMF-10 is indicated in Table 21. The oligonucleotide sequences used as primers are boxed and the oligonucleotide sequence used as a probe is underlined.

TABLE 21
AMF-10 (G55707 A) Sequence Input for TaqMan Analysis

| | | |
|---|---|---|
| NN\overline{\|GACTTACTCCATCGCTGAGAAGCT\|}GGGCATCAATGCCAOCTTTTTCCAGTCTTCCAAG\overline{\|TCGGCTAATACGATCACCAG\|} | 80 | (SEQ ID NO.57) |
| T Y S I A E K L G I N A S F F Q S S K S A N T I T S | (SEQ ID NO.20) | |
| \overline{\|C\|}TTTGTAGACAGGGGACTAGNN | 102 | |
| F V D R G L | | |

The following primer and probe sequences were used for TaqMan analysis of AMF-10.

| | |
|---|---|
| Ag 191 (F): 5′-GACTTACTCCATCGCTGAGAAGCT-3′ | (SEQ ID NO.58) |
| Ag 191 (R): 5′-GCTGGTGATCGTATTAGCCGA-3′ | (SEQ ID NO.59) |
| Ag 191 (P): FAM-5′-CATCAATGCCAGCTTTTTCCAGTCTTCC-3′-TAMRA | (SEQ ID NO.60) |

### Example 2

### Quantitation of AMFX Gene Expression Using TaqMan Analysis

The quantitative expression patterns of clones AMF-1-10 were assessed in a large number of normal and tumor sample cells and cell lines by real time quantitative PCR (TaqMan®) performed on a Perkin-Elmer Biosystems ABI PRISM® 7700 Sequence Detection System. Table 21 shows the expression patterns of AMF-1, AMF-2, AMF-4, and AMF-6.

TABLE 21
AMF-X gene expression in cells and tissues.

| | AFM-1 | AMF-2 | AMF-6 | AMF-4 |
|---|---|---|---|---|
| Normal & Tumor Tissues | Relative Expression (%) | | | |
| Endothelial cells | 0.00 | 4.97 | 17.31 | 0.00 |
| Endothelial cells (treated) | 0.00 | 4.30 | 5.15 | 0.00 |
| Pancreas | 0.00 | 3.06 | 13.03 | 14.66 |
| Pancreatic ca. CAPAN 2 | 0.00 | 23.98 | 10.73 | 0.00 |
| Adipose | 2.66 | 39.78 | 62.85 | 0.00 |
| Adrenal gland | 0.00 | 8.19 | 4.30 | 0.00 |
| Thyroid | 7.38 | 6.08 | 6.56 | 11.27 |
| Salivary gland | 5.87 | 4.09 | 15.60 | 13.58 |
| Pituitary gland | 0.00 | 10.22 | 2.29 | 0.00 |
| Brain (fetal) | 100.00 | 8.96 | 1.08 | 0.00 |
| Brain (whole) | 3.00 | 3.74 | 0.12 | 0.00 |
| Brain (amygdala) | 0.80 | 1.66 | 0.19 | 0.00 |
| Brain (cerebellum) | 1.44 | 10.51 | 6.75 | 0.00 |
| Brain (hippocampus) | 2.80 | 1.18 | 0.00 | 0.00 |
| Brain (hypothalamus) | 5.63 | 3.42 | 1.07 | 6.79 |
| Brain (substantia nigra) | 7.33 | 3.52 | 0.26 | 0.01 |
| Brain (thalamus) | 2.01 | 2.70 | 0.46 | 0.00 |
| Spinal cord | 1.18 | 3.96 | 1.69 | 0.00 |
| CNS ca. (glio/astro) U87-MG | 0.00 | 23.98 | 0.00 | 0.00 |
| CNS ca. (glio/astro) U-118-MG | 0.00 | 24.83 | 33.22 | 0.00 |
| CNS ca. (astro) SW1783 | 0.00 | 17.08 | 37.37 | 0.00 |
| CNS ca.* (neuro; met) SK-N-AS | 0.00 | 17.56 | 0.00 | 0.00 |
| CNS ca. (astro) SF-539 | 0.00 | 27.36 | 3.54 | 0.00 |
| CNS ca. (astro) SNB-75 | 0.00 | 65.07 | 4.07 | 0.00 |
| CNS ca. (glio) SNB-19 | 2.68 | 53.59 | 0.00 | 0.00 |
| CNS ca. (glio) U251 | 0.00 | 26.79 | 0.23 | 0.00 |
| CNS ca. (glio) SF-295 | 0.00 | 33.45 | 15.71 | 3.33 |
| Heart | 0.00 | 4.54 | 15.18 | 0.00 |
| Skeletal muscle | 0.00 | 1.91 | 0.32 | 0.00 |
| Bone marrow | 0.00 | 1.73 | 6.34 | 0.00 |
| Thymus | 1.86 | 18.95 | 56.64 | 0.00 |
| Spleen | 0.00 | 5.08 | 9.09 | 0.29 |
| Lymph node | 0.00 | 6.04 | 32.09 | 2.19 |
| Colon (ascending) | 0.81 | 3.24 | 0.21 | 0.01 |
| Stomach | 0.00 | 11.99 | 18.82 | 26.24 |
| Small intestine | 0.00 | 8.66 | 9.02 | 2.84 |
| Colon ca. SW480 | 0.00 | 1.85 | 0.00 | 0.00 |
| Colon ca.* (SW480 met)SW620 | 0.18 | 2.42 | 0.00 | 10.88 |
| Colon ca. HT29 | 0.00 | 1.75 | 0.87 | 0.00 |
| Colon ca. HCT-116 | 2.72 | 10.37 | 2.47 | 0.00 |
| Colon ca. CaCo-2 | 21.92 | 21.76 | 3.93 | 0.00 |
| Colon Ca. HCT-15 | 1.99 | 4.97 | 4.61 | 9.67 |
| Colon ca. HCC-2998 | 0.00 | 1.15 | 11.58 | 0.00 |
| Gastric ca.* (liver met) NCI-N87 | 91.38 | 3.06 | 85.86 | 100.00 |
| Bladder | 0.00 | 15.93 | 29.32 | 0.00 |
| Trachea | 0.00 | 7.03 | 32.09 | 40.61 |
| Kidney | 7.59 | 8.90 | 8.66 | 0.02 |
| Kidney (fetal) | 46.65 | 55.86 | 32.09 | 2.19 |
| Renal ca. 786-0 | 0.00 | 96.59 | 28.13 | 0.00 |
| Renal ca. A498 | 0.00 | 65.52 | 40.90 | 0.00 |
| Renal ca. RXF 393 | 0.00 | 27.74 | 18.82 | 0.00 |
| Renal ca. ACHN | 0.00 | 65.07 | 5.79 | 0.00 |
| Renal ca. UO-31 | 0.00 | 41.75 | 17.31 | 0.00 |
| Renal ca. TK-10 | 0.00 | 56.64 | 8.84 | 0.00 |
| Liver | 0.13 | 3.30 | 11.99 | 2.76 |
| Liver (fetal) | 0.05 | 2.35 | 2.32 | 0.00 |
| Liver ca. (hepatoblast) HepG2 | 14.66 | 0.02 | 0.00 | 0.27 |
| Lung | 7.75 | 8.02 | 42.93 | 0.04 |
| Lung (fetal) | 81.79 | 11.91 | 100.00 | 0.01 |
| Lung ca. (small cell) LX-1 | 1.61 | 1.35 | 11.34 | 48.97 |
| Lung ca. (small cell) NCI-H69 | 0.04 | 4.15 | 0.00 | 0.00 |
| Lung ca. (s.cell var.) SHP-77 | 0.32 | 0.36 | 0.00 | 0.00 |
| Lung ca. (large cell)NCI-H460 | 0.00 | 26.98 | 0.41 | 0.00 |
| Lung ca. (non-sm. cell) A549 | 0.13 | 7.13 | 0.78 | 0.00 |
| Lung ca. (non-s.cell) NCI-H23 | 0.00 | 7.08 | 2.38 | 0.00 |
| Lung ca (non-s.cell) HOP-62 | 0.00 | 15.82 | 1.30 | 0.00 |
| Lung ca. (non-s.cl)NCI-H522 | 1.31 | 5.37 | 15.28 | 0.00 |
| Lung ca. (squam.) SW 900 | 0.00 | 17.08 | 17.08 | 0.00 |
| Lung ca. (squam.) NCI-H596 | 0.02 | 8.66 | 0.00 | 0.00 |
| Mammary gland | 0.23 | 45.06 | 55.10 | 31.86 |
| Breast ca.* (pl. effusion) MCF-7 | 0.00 | 0.00 | 4.15 | 8.30 |
| Breast ca.* (pl.ef) MDA-MB-231 | 0.00 | 15.07 | 0.83 | 0.00 |
| Breast ca.* (pl. effusion) T47D | 3.61 | 5.33 | 8.72 | 57.83 |
| Breast ca. BT-549 | 0.00 | 65.07 | 97.94 | 0.00 |
| Breast ca. MDA-N | 0.00 | 25.70 | 0.00 | 0.00 |
| Ovary | 0.28 | 39.50 | 14.97 | 3.52 |
| Ovarian ca. OVCAR-3 | 7.48 | 32.31 | 1.24 | 0.21 |
| Ovarian ca. OVCAR-4 | 8.78 | 32.99 | 1.03 | 6.93 |
| Ovarian ca. OVCAR-5 | 0.00 | 35.60 | 36.10 | 0.73 |
| Ovarian ca. OVCAR-8 | 0.00 | 20.03 | 13.58 | 1.04 |
| Ovarian ca. IGROV-1 | 0.04 | 47.96 | 13.68 | 0.00 |
| Ovarian ca.* (ascites) SK-OV-3 | 0.00 | 47.63 | 3.87 | 0.00 |
| Myometrium | 1.03 | 23.49 | 19.08 | 0.16 |
| Uterus | 8.48 | 9.94 | 19.08 | 0.29 |
| Placenta | 0.00 | 23.82 | 4.97 | 0.05 |
| Prostate | 0.29 | 6.75 | 46.98 | 0.65 |
| Prostate ca.* (bone met)PC-3 | 0.00 | 37.63 | 7.86 | 0.00 |
| Testis | 6.25 | 23.82 | 17.19 | 0.00 |
| Melanoma Hs688(A).T | 0.00 | 23.00 | 44.44 | 0.00 |
| Melanoma* (met) Hs688(B).T | 0.00 | 25.35 | 38.69 | 0.00 |
| Melanoma UACC-62 | 0.00 | 23.00 | 0.02 | 0.00 |
| Melanoma M14 | 0.00 | 36.10 | 1.13 | 0.00 |
| Melanoma LOX IMVI | 0.00 | 100.00 | 0.01 | 0.00 |
| Melanoma* (met) SK-MEL-5 | 0.00 | 10.88 | 0.10 | 0.00 |
| Melanoma SK-MEL-28 | 0.00 | 79.00 | 11.91 | 0.00 |
| Melanoma UACC-257 | 0.00 | 0.00 | 0.00 | 0.00 |
| | TM | TM | TM | TM |
| | 407F | 418 F | 371 | 416 F |

The quantitative expression patterns of clones AMF-1-10 were assessed in a large number of normal and tumor sample cells and cell lines by real time quantitative PCR (TaqMan®) performed on a Perkin-Elmer Biosystems ABI PRISM® 7700 Sequence Detection System. Table 22 shows the expression patterns of AMF-3, AMF-7, AMF-8, and AMF-10.

TABLE 22
AMF-X gene expression in cells and tissues.

| | AMF-10 | AMF-8 | AMF-3 | AMF-7 |
|---|---|---|---|---|
| Normal & Tumor Tissues | Relative Expression (%) | | | |
| Endothelial cells | 0.00 | 0.58 | 0.02 | 0.39 |
| Endothelial cells (treated) | 0.00 | 0.23 | 0.09 | 0.57 |
| Pancreas | 0.08 | 3.15 | 0.17 | 0.21 |
| Pancreatic ca. CAPAN 2 | 0.00 | 0.62 | 0.10 | 1.64 |
| Adipose | 0.47 | 8.13 | 2.47 | 0.00 |
| Adrenal gland | 0.00 | 2.47 | 0.64 | 0.51 |
| Thyroid | 0.00 | 7.54 | 1.31 | 0.53 |
| Salivary gland | 0.00 | 4.54 | 1.69 | 0.45 |
| Pituitary gland | 0.01 | 19.75 | 0.04 | 0.08 |
| Brain (fetal) | 0.00 | 20.03 | 41.18 | 3.35 |
| Brain (whole) | 0.00 | 37.89 | 0.01 | 3.52 |
| Brain (amygdala) | 0.00 | 20.45 | 15.28 | 0.96 |
| Brain (cerebellum) | 0.00 | 100.00 | 100.00 | 1.92 |
| Brain (hippocampus) | 0.00 | 22.53 | 28.52 | 6.61 |
| Brain (hypothalamus) | 0.00 | 76.31 | 4.24 | 1.28 |
| Brain (substantia nigra) | 0.00 | 30.57 | 22.69 | 1.67 |
| Brain (thalamus) | 0.00 | 29.32 | 9.21 | 2.43 |
| Spinal cord | 0.00 | 35.11 | 1.76 | 0.59 |
| CNS ca. (glio/astro) U87-MG | 0.00 | 8.66 | 0.01 | 1.49 |
| CNS ca. (glio/astro) U-118-MG | 100.00 | 2.18 | 0.01 | 3.52 |
| CNS ca. (astro) SW1783 | 4.15 | 1.61 | 0.00 | 1.16 |
| CNS ca.* (neuro; met) SK-N-AS | 0.00 | 38.42 | 0.95 | 9.41 |
| CNS ca. (astro) SF-539 | 0.00 | 3.61 | 0.00 | 1.12 |
| CNS ca. (astro) SNB-75 | 0.00 | 23.98 | 0.00 | 1.45 |
| CNS ca. (glio) SNB-19 | 0.00 | 33.68 | 0.48 | 1.03 |
| CNS ca. (glio) U251 | 0.18 | 9.41 | 0.12 | 0.88 |
| CNS ca. (glio) SF-295 | 0.00 | 11.83 | 0.00 | 0.41 |
| Heart | 0.00 | 11.27 | 0.36 | 0.25 |
| Skeletal muscle | 0.00 | 0.54 | 0.48 | 0.11 |
| Bone marrow | 0.00 | 1.88 | 0.06 | 1.35 |
| Thymus | 0.00 | 6.84 | 0.66 | 3.77 |
| Spleen | 0.00 | 8.25 | 0.12 | 0.42 |
| Lymph node | 0.00 | 2.78 | 0.11 | 0.50 |
| Colon (ascending) | 0.00 | 2.90 | 2.12 | 0.23 |
| Stomach | 0.00 | 9.02 | 1.23 | 0.39 |
| Small intestine | 0.00 | 8.30 | 0.42 | 1.73 |
| Colon ca. SW480 | 0.00 | 0.32 | 0.02 | 1.60 |
| Colon ca* (SW480 met)SW620 | 0.00 | 0.52 | 0.18 | 3.59 |
| Colon ca. HT29 | 0.00 | 0.49 | 0.05 | 2.98 |
| Colon ca. HCT-116 | 0.00 | 1.15 | 3.26 | 58.64 |
| Colon ca. CaCo-2 | 0.00 | 5.40 | 2.21 | 4.77 |
| Colon ca. HCT-15 | 0.00 | 1.39 | 0.32 | 2.74 |
| Colon ca. HCC-2998 | 0.00 | 0.93 | 0.15 | 3.96 |
| Gastric ca.* (liver met) | 0.00 | 1.27 | 9.61 | 2.94 |
| NCI-N87 | | | | |
| Bladder | 0.13 | 5.79 | 1.50 | 0.00 |
| Trachea | 0.00 | 8.54 | 0.77 | 1.91 |
| Kidney | 0.00 | 5.11 | 1.10 | 0.20 |
| Kidney (fetal) | 0.00 | 22.69 | 5.11 | 3.13 |
| Renal ca. 786-0 | 0.00 | 1.10 | 0.01 | 2.54 |
| Renal ca. A498 | 0.00 | 1.30 | 0.00 | 2.19 |
| Renal ca. RXF 393 | 0.00 | 1.04 | 0.00 | 0.60 |
| Renal ca. ACHN | 0.00 | 0.44 | 0.00 | 1.33 |
| Renal ca. UO-31 | 0.00 | 0.85 | 0.04 | 0.56 |
| Renal ca. TK-10 | 0.00 | 1.17 | 0.12 | 2.94 |
| Liver | 0.00 | 2.76 | 0.14 | 2.78 |
| Liver (fetal) | 0.00 | 2.24 | 0.22 | 3.52 |
| Liver ca. (hepatoblast) HepG2 | 0.00 | 1.29 | 0.71 | 1.70 |
| Lung | 0.00 | 1.41 | 0.56 | 0.01 |
| Lung (fetal) | 0.00 | 11.27 | 16.27 | 1.92 |
| Lung ca. (small cell) LX-1 | 0.00 | 0.83 | 0.32 | 3.24 |
| Lung ca. (small cell) NCI-H69 | 0.00 | 8.84 | 1.51 | 5.48 |
| Lung ca. (s.cell var.) SHP-77 | 0.00 | 1.88 | 6.98 | 100.00 |
| Lung ca. (large cell)NCI-H460 | 0.00 | 1.39 | 43.53 | 6.93 |
| Lung ca. (non-sm. Cell) A549 | 0.00 | 1.41 | 0.05 | 0.84 |
| Lung ca. (non-s.cell)NCI-H23 | 0.00 | 1.10 | 0.84 | 2.21 |
| Lung ca (non-s.cell) HOP-62 | 0.00 | 1.24 | 0.09 | 0.23 |
| Lung ca. (non-s.cl)NCI-H522 | 0.00 | 2.35 | 0.40 | 15.39 |
| Lung ca.(squam.)SW 900 | 0.00 | 1.51 | 0.78 | 3.37 |
| Lung ca. (squam.)NCI-H596 | 0.00 | 4.09 | 1.21 | 7.80 |
| Mammary gland | 0.00 | 17.31 | 1.18 | 0.43 |
| Breast ca.* (pl. effusion) MCF-7 | 0.00 | 1.87 | 0.08 | 6.75 |
| Breast ca.* (pl.ef) | 0.00 | 0.76 | 0.00 | 1.71 |
| MDA-MB-231 | | | | |
| Breast ca.* (pl. effusion) T47D | 0.00 | 0.98 | 0.94 | 1.47 |
| Breast ca. BT-549 | 0.00 | 2.74 | 0.19 | 18.30 |
| Breast ca. MDA-N | 0.00 | 4.61 | 0.17 | 13.68 |
| Ovary | 0.00 | 3.00 | 0.63 | 0.68 |
| Ovarian ca. OVCAR-3 | 0.00 | 0.61 | 1.57 | 1.63 |
| Ovarian ca. OVCAR-4 | 0.00 | 1.00 | 0.80 | 1.17 |
| Ovarian ca. OVCAR-5 | 0.00 | 0.75 | 0.45 | 4.97 |
| Ovarian ca. OVCAR-8 | 0.00 | 0.80 | 0.14 | 2.19 |
| Ovarian ca. IGROV-1 | 0.00 | 0.50 | 0.09 | 1.10 |
| Ovarian ca.* (ascites) SK-OV-3 | 0.03 | 0.63 | 0.10 | 3.67 |
| Myometrium | 0.00 | 13.40 | 1.34 | 0.07 |
| Uterus | 0.00 | 6.52 | 1.36 | 0.44 |
| Placenta | 3.59 | 21.02 | 0.37 | 2.19 |
| Prostate | 0.00 | 27.36 | 1.16 | 0.40 |
| Prostate ca.* (bone met)PC-3 | 0.00 | 1.81 | 7.48 | 18.05 |
| Testis | 0.36 | 56.64 | 1.82 | 21.76 |
| Melanoma Hs688(A).T | 0.00 | 1.62 | 0.00 | 0.33 |
| Melanoma* (met) Hs688(B).T | 0.20 | 0.94 | 0.08 | 0.04 |
| Melanoma UACC-62 | 0.00 | 0.54 | 0.00 | 0.12 |
| MelanomaM14 | 0.00 | 1.94 | 0.56 | 1.25 |
| Melanoma LOX IMVI | 0.00 | 2.12 | 0.10 | 33.68 |
| Melanoma* (met) SK-MEL-5 | 0.00 | 0.96 | 0.16 | 2.21 |
| Melanoma SK-MEL-28 | 0.00 | 1.81 | 0.01 | 4.04 |
| Melanoma UACC-257 | | 0.00 | 0.00 | 9.02 |
| | TM | TM | TM | TM |
| | 361 F | 415 T | 208 F | 221 F |

TaqMan expression analysis was also performed on AMF-5 and AMF-9.

### Equivalents

Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18 and 20;
(b) a variant of a mature form of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18 and 20; and
(d) a variant of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18 and 20.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17 and 19.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18 and 20;
(b) a variant of a mature form of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18 and 20;
(d) a variant of an amino acid sequence selected from the group consisting of SEQ ID NOS:2, 4, 6, 8, 10,12,14,16,18 and 20, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18 and 20, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1,3,5,7,9, 11,13,15,17 and 19.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9,11,13,15, 17 and 19;
(b) a nucleotide sequence differing by one or more nucleotides from a nucleotide sequence selected from the group consisting of SEQ ID NOS: 1, 3, 5, 7, 9, 11, 13, 15, 17 and 19, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17 and 19, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a AMFX-associated disorder, said method comprising administering to a subject in which such treatment or prevention is desired the polypeptide of claim 1 in an amount sufficient to treat or prevent said AMFX-associated disorder in said subject.

27. The method of claim 26 wherein the disorder is selected from the group consisting of disorders related to cell signal processing, cell adhesion or migration pathway modulation, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, and cellular responses to external stimuli, and other diseases, disorders and conditions of the like.

28. The method of claim 26 wherein the disorder is related to cell signal processing and metabolic pathway modulation.

29. The method of claim 26, wherein said subject is a human.

30. A method of treating or preventing a AMFX-associated disorder, said method comprising administering to a subject in which such treatment or prevention is desired the nucleic acid of claim 5 in an amount sufficient to treat or prevent said AMFX-associated disorder in said subject.

31. The method of claim 30 wherein the disorder is selected from the group consisting of consisting of disorders related to cell signal processing, cell adhesion or migration pathway modulation, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, and cellular responses to external stimuli, and other diseases, disorders and conditions of the like.

32. The method of claim 30 wherein the disorder is related to cell signal processing and metabolic pathway modulation.

33. The method of claim 30, wherein said subject is a human.

34. A method of treating or preventing a AMFX-associated disorder, said method comprising administering to a subject in which such treatment or prevention is desired the antibody of claim 15 in an amount sufficient to treat or prevent said AMFX-associated disorder in said subject

35. The method of claim 34 wherein the disorder is selected from the group consisting of disorders related to cell signal processing, cell adhesion or migration pathway modulation, chemoresistance, radiotherapy resistance, survival in trophic factor limited secondary tissue site microenvironments, connective tissue disorders, tissue remodeling, oncogenesis, cancer of the breast, ovary, cervix, prostate, endometrium, stomach, colon, lung, bladder, kidney, brain, and soft-tissue, cellular transformation, developmental tissue remodeling, inflammation, blood clot formation and resorption, hematopoiesis, angiogenesis, multidrug resistance related to organic anion transporters, malignant disease progression, autocrine and paracrine regulation of cell growth, and cellular responses to external stimuli, and other diseases, disorders and conditions of the like.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to cancers.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18 and 20, or a biologically active fragment thereof.

49. A method of treating a pathological state in a mammal, the method comprising administering to the mammal the antibody of claim 15 in an amount sufficient to alleviate the pathological state.
